(19) United States
(12) Patent Application Publication
(10) Pub. No.: US 2023/0416287 A1
(43) Pub. Date: Dec. 28, 2023

(54) Chemical Compounds

(71) Applicant: GLAXOSMITHKLINE INTELLECTUAL PROPERTY DEVELOPMENT LIMITED, Brentford (GB)

(72) Inventors: Jie GUANG, Collegeville, PA (US); Jay M. MATTHEWS, Collegeville, PA (US); Alan T. PRICE, Collegeville, PA (US); Jared Troy SPLETSTOSER, Collegeville, PA (US)

(73) Assignee: GLAXOSMITHKLINE INTELLECTUAL PROPERTY DEVELOPMENT LIMITED, Brentford (GB)

(21) Appl. No.: 18/330,569

(22) Filed: Jun. 7, 2023

Related U.S. Application Data
(60) Provisional application No. 63/350,640, filed on Jun. 9, 2022.

(51) Int. Cl. C07F 9/6561; C07F 9/6558

(52) U.S. Cl. CPC C07F 9/6561; C07F 9/65583

(57) ABSTRACT

Provided are compounds and pharmaceutical compositions and uses thereof for inhibiting Naᵥ1.8 voltage-gated sodium ion channels and treating Naᵥ1.8 mediated diseases, such as pain and pain-associated diseases, and cardiovascular diseases.

### FIELD OF THE INVENTION

The invention relates to Naᵥ1.8 inhibitor compounds or pharmaceutically acceptable salts or tautomer forms thereof, corresponding pharmaceutical compositions or formulations, methods or processes of compound preparation, methods, compounds for use in, uses for and/or combination therapies for treating pain and pain-associated diseases and cardiovascular diseases.

### BACKGROUND OF THE INVENTION

Pain is a protective mechanism by which animals avoid potential tissue damage, however there are numerous disease indications in which pain outlives its usefulness and becomes a disabling burden. Indications in which pain outlives its usefulness can be broadly categorized as those in which nerve damage or injury is the trigger (neuropathic pain), those in which an inflammatory response or metabolic dysregulation sensitizes the pain response (inflammatory pain) and those in which an injury or surgical procedure results in a short-term elevation of pain response (post-operative/ambulatory pain).

Voltage-gated sodium channels underlie electrical signaling in all excitable tissues by setting the threshold and underlying the upstroke of action potentials. There are nine distinct isoforms of voltage-gated sodium channels. Those designated Naᵥ1.1, Naᵥ1.7, Naᵥ1.8 and Naᵥ1.9 are principally expressed on peripheral nerves where they control neuronal excitability. Naᵥ1.5 is the main sodium channel isoform expressed in cardiac myocytes, Naᵥ1.4 is expressed and functions in skeletal muscle, whereas Naᵥ1.1, Naᵥ1.2, Naᵥ1.3 and Naᵥ1.6 are widely expressed in the central nervous system (CNS) and to an extent in the peripheral nervous system. The principal role of these nine voltage-gated sodium channels is comparable in that they control sodium influx into cells, but their biophysical properties vary which greatly influences the physiological profile of their respective cell type (Catterall, 2012).

Currently, non-selective sodium channel inhibitors are utilized clinically as anti-arrhythmic and anti-seizure therapies, these include lidocaine, carbamazepine, amitriptyline and mexiletine. However, as these agents exhibit a lack of selectivity between the different sodium channel isoforms, their therapeutic utility is greatly reduced due to adverse side effects, largely mediated by activity in the CNS and heart. This has stimulated efforts to develop novel medicines which are selective for specific sodium channel isoforms in order to avoid side effects in the CNS and cardiovascular system.

The Naᵥ1.8 channel is expressed in neurons of the dorsal root ganglia (DRG) and highly expressed in the small diameter neurons of this tissue which form pain sensing C- and Aδ-nerve fibers (Abrahamsen, 2008; Amaya, 2000; Novakovic, 1998). The channel was proposed as a therapeutic target for analgesia as soon as it was originally cloned from rat DRG (Akopian, 1996) due to its prominent physiological role in this tissue type and restricted expression profile. Naᵥ1.8 was subsequently identified, cloned and characterized from human DRG tissue (Rabart 1998). The closest molecular relative of Naᵥ1.8 is Naᵥ1.5 which shares a sequence homology of ˜60%. Naᵥ1.8 was previously known as SNS (sensory neuron sodium channel), PN3 (peripheral nerve sodium channel type 3), and as it exhibits characteristic pharmacological properties in its resistance to block by tetrodotoxin, it is also described as a TTX-resistant sodium channel.

Support for Naᵥ1.8 as a therapeutic target for pain indications comes from several sources. Naᵥ1.8 has been shown to conduct the majority of current during upstroke of the action potential in DRG neurons (Blair & Bean, 2002) and due to its rate of re-priming is also critical for the ability of these neurons to fire repetitively (Blair and Bean, 2003). Increased expression and function of Naᵥ1.8 has been reported in response to painful stimuli such as inflammatory mediators (England 1996 & Gold 1996), nerve damage (Roza 2003 & Ruangsri 2011), and within painful neuromas (Black 2008 & Coward 2000). Knockout of the gene encoding Nav1.8 in mice resulted in a reduced pain phenotype in particular to inflammatory challenges (Akopian 1999). Knockdown of the mRNA encoding Naᵥ1.8 also resulted in reduced painful phenotypes in rodent models, particularly in neuropathic models (Lai 2002). Pharmacological intervention via selective small molecule inhibitors has demonstrated efficacy in rodent models of inflammatory pain as well as neuropathic pain (Jarvis 2007 & Payne 2015). Supporting genetic evidence for Naᵥ1.8 is also present in patients with chronic neuropathic pain where multiple gain of function mutations has been reported to be causative in episodic painful neuropathies and small fiber neuropathies (Faber 2012, Han 2014 & Eijkenboom 2018).

### SUMMARY OF THE INVENTION

Accordingly, there is a need for the development of novel compounds, particularly Naᵥ1.8 inhibitor compounds that have improved solubility and are thus more advantageous for alternative routes of administration, such as intravenous administration, for use in the treatment of pain and pain associated diseases, and cardiovascular diseases. The invention satisfies this need by providing compounds with Naᵥ1.8 inhibitory activity and prodrugs of compounds with Naᵥ1.8 inhibitory activity and uses of such compounds and prodrugs in the treatment of pain and pain associated diseases, and cardiovascular diseases. The prodrugs of the invention in particular have improved solubility as compared to their respective parent compounds, and thus can be useful for intravenous (IV) administration and treatment of pain and pain associated diseases in which IV administration may be beneficial or preferred, such as in the treatment of acute pain.

In one aspect, provided is a compound selected from the group consisting of:

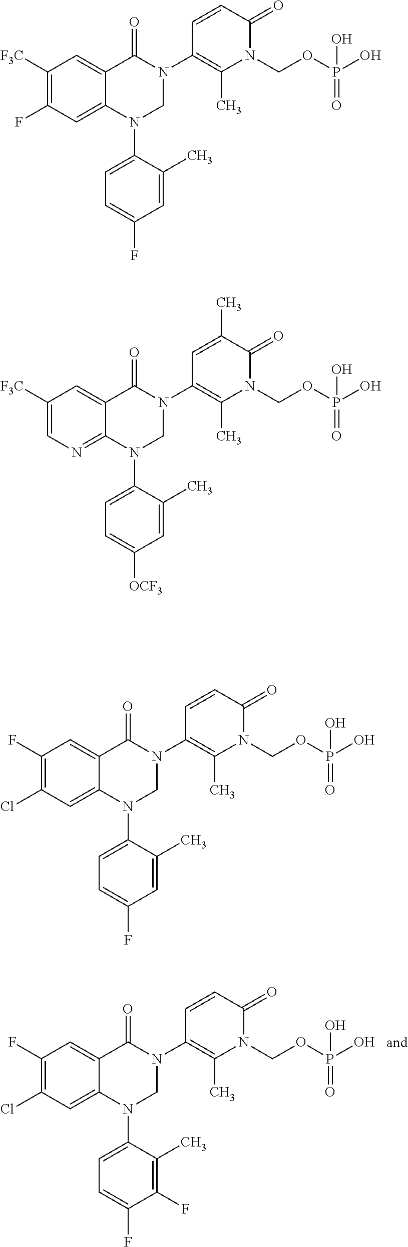 [SMILES: Cc1cc(F)ccc1N1CN(c2ccc(=O)n(COP(=O)(O)O)c2C)C(=O)c2cc(C(F)(F)F)c(F)cc21 ; Cc1cc(OC(F)(F)F)ccc1N1CN(c2cc(C)c(=O)n(COP(=O)(O)O)c2C)C(=O)c2cc(C(F)(F)F)cnc21 ; Cc1cc(F)ccc1N1CN(c2ccc(=O)n(COP(=O)(O)O)c2C)C(=O)c2cc(F)c(Cl)cc21 ; Cc1c(N2CN(c3ccc(=O)n(COP(=O)(O)O)c3C)C(=O)c3cc(F)c(Cl)cc32)ccc(F)c1F ; "and"]

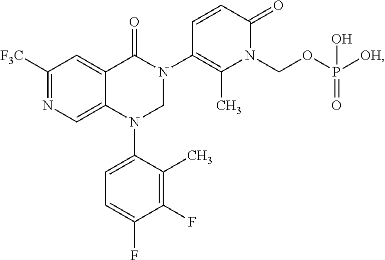 [SMILES: Cc1c(N2CN(c3ccc(=O)n(COP(=O)(O)O)c3C)C(=O)c3cc(C(F)(F)F)ncc32)ccc(F)c1F]

or a pharmaceutically acceptable salt thereof.

In another aspect, provided is a compound selected from the group consisting of:

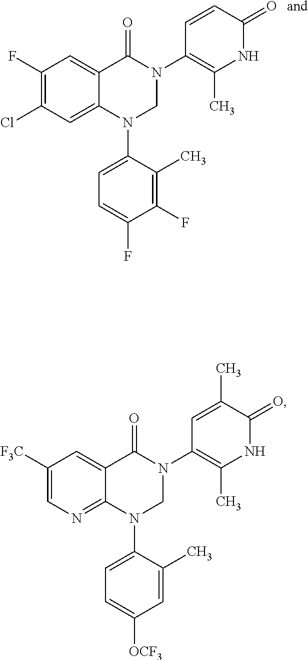 [SMILES: Cc1[nH]c(=O)ccc1N1CN(c2ccc(F)c(F)c2C)c2cc(Cl)c(F)cc2C1=O ; "and" ; Cc1cc(OC(F)(F)F)ccc1N1CN(c2cc(C)c(=O)[nH]c2C)C(=O)c2cc(C(F)(F)F)cnc21]

or a tautomer thereof, or a pharmaceutically acceptable salt thereof.

In another aspect, provided is a pharmaceutical composition comprising a compound, or tautomer thereof, or pharmaceutically acceptable salt thereof of the invention, and a pharmaceutically acceptable excipient.

In another aspect, provided is a method of treatment of pain or a pain-associated disease in a human in need thereof, the method comprising administering to the human a compound, or tautomer thereof, or pharmaceutically acceptable salt thereof of the invention, or a pharmaceutical composition of the invention.

In another aspect, provided is a method of treatment of atrial fibrillation in a human in need thereof, the method comprising administering to the human a compound, or tautomer thereof, or pharmaceutically acceptable salt thereof of the invention, or a pharmaceutical composition of the invention.

In another aspect, provided is a compound, or tautomer thereof, or pharmaceutically acceptable salt thereof of the invention, or a pharmaceutical composition of the invention for use in therapy.

In another aspect, provided is a compound, or tautomer thereof, or pharmaceutically acceptable salt thereof of the invention, or a pharmaceutical composition of the invention for use in treatment of pain or a pain-associated disease.

In another aspect, provided is a compound, or tautomer thereof, or pharmaceutically acceptable salt thereof of the invention, or a pharmaceutical composition of the invention for use in treatment of atrial fibrillation.

In another aspect, provided is use of a compound, or tautomer thereof, or pharmaceutically acceptable salt thereof of the invention, or a pharmaceutical composition of the invention in the manufacture of a medicament for treatment of pain or a pain-associated disease.

In another aspect, provided is use of a compound, or tautomer thereof, or pharmaceutically acceptable salt thereof of the invention, or a pharmaceutical composition of the invention in the manufacture of a medicament for treatment of atrial fibrillation.

### BRIEF DESCRIPTION OF THE FIGURES

FIG. 1 shows an X-ray powder diffraction (XRPD) pattern for crystalline (5-(7-chloro-1-(3,4-difluoro-2-methylphenyl)-6-fluoro-4-oxo-1,4-dihydroquinazolin-3(2H)-yl)-6-methyl-2-oxopyridin-1(2H)-yl)methyl dihydrogen phosphate prepared according to Route 1.

FIG. 2 shows an X-ray powder diffraction (XRPD) pattern for crystalline (5-(7-chloro-1-(3,4-difluoro-2-methylphenyl)-6-fluoro-4-oxo-1,4-dihydroquinazolin-3(2H)-yl)-6-methyl-2-oxopyridin-1(2H)-yl)methyl dihydrogen phosphate prepared according to Route 2.

FIG. 3 shows an X-ray powder diffraction (XRPD) pattern for crystalline (5-(7-chloro-1-(3,4-difluoro-2-methylphenyl)-6-fluoro-4-oxo-1,4-dihydroquinazolin-3(2H)-yl)-6-methyl-2-oxopyridin-1(2H)-yl)methyl dihydrogen phosphate prepared according to Route 3.

FIG. 4 shows an X-ray powder diffraction (XRPD) pattern for crystalline (5-(7-chloro-1-(3,4-difluoro-2-methylphenyl)-6-fluoro-4-oxo-1,4-dihydroquinazolin-3(2H)-yl)-6-methyl-2-oxopyridin-1(2H)-yl)methyl dihydrogen phosphate prepared according to Route 4.

FIG. 5 shows an X-ray powder diffraction (XRPD) pattern for crystalline di-tert-butyl ((5-(7-chloro-1-(3,4-difluoro-2-methylphenyl)-6-fluoro-4-oxo-1,4-dihydroquinazolin-3(2H)-yl)-6-methyl-2-oxopyridin-1(2H)-yl)methyl) phosphate (seed material).

### DETAILED DESCRIPTION OF THE INVENTION

Various publications, articles and patents are cited or described in the background and throughout the specification. Discussion of documents, acts, materials, devices, articles or the like which has been included in the present specification is for the purpose of providing context for the disclosure. Such discussion is not an admission that any or all of these matters form part of the prior art with respect to the disclosure.

Unless defined otherwise, all technical and scientific terms used herein have the same meaning as commonly understood to one of ordinary skill in the art to which this invention pertains. Otherwise, certain terms used herein have the meanings as set forth in the specification.

As used herein and in the appended claims, the singular forms “a,” “an,” and “the” include plural reference unless the context clearly dictates otherwise.

The definitions for the various groups and substituent groups of any of the Formulas disclosed herein, or a pharmaceutically acceptable salt thereof provided throughout the specification are intended to particularly describe each compound species disclosed herein, individually, as well as groups of one or more compound species.

The term “alkyl” refers to a saturated hydrocarbon radical, straight or branched, having the specified number of carbon atoms. For example, the term “(C₁-C₆)alkyl” refers to an alkyl group having 1 to 6 carbon atoms and the term “(C₁-C₃)alkyl” refers to an alkyl group having 1 to 3 carbon atoms. Exemplary alkyls include, but are not limited to, methyl, ethyl, n-propyl, isopropyl, n-butyl, isobutyl, s-butyl, t-butyl, pentyl, and hexyl. In some embodiments, “Me” refers to a methyl group.

The terms “halogen” and “halo” represent chloro (—Cl), fluoro (—F), bromo (—Br), or iodo (—I) substituents.

When the term “alkyl” is used in combination with other substituent groups, such as “haloalkyl”, the term “alkyl” is intended to encompass a divalent straight or branched chain hydrocarbon radical, wherein the point of attachment is through the alkyl moiety.

The term “haloalkyl” refers to a radical having one more halogen atoms, which may be the same or different, at one or more carbon atoms of an alkyl moiety having the specified number of carbon atoms, which is a straight or branched chain carbon radical. For example, the term “halo(C₁-C₆)alkyl” refers to a radical having one or more halogen atoms, which may be the same or different, at one or more carbon atoms of an alkyl moiety having 1 to 6 carbon atoms, which is a straight or branched chain carbon radical. Examples of “haloalkyl” groups include, but are not limited to, —CH₂F (fluoromethyl), —CHF₂ (difluoromethyl), —CF₃ (trifluoromethyl), —CCl₃ (trichloromethyl), 1,1-difluoroethyl, 2-fluoro-2-methylpropyl, 2,2-difluoropropyl, 2,2,2-trifluoroethyl, and hexafluoroisopropyl.

Compounds

In one aspect of the invention, provided is a compound selected from the group consisting of:

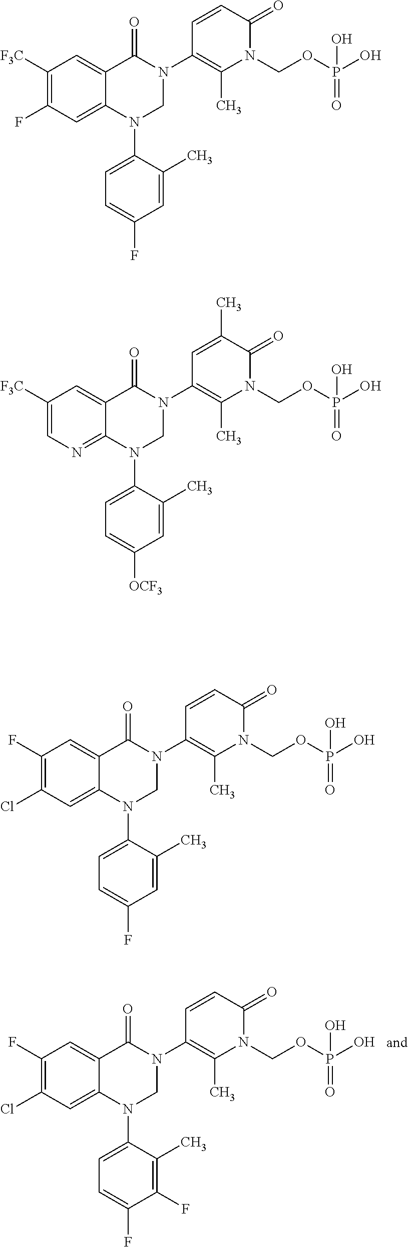 [SMILES: Cc1cc(F)ccc1N1CN(c2ccc(=O)n(COP(=O)(O)O)c2C)C(=O)c2cc(C(F)(F)F)c(F)cc21 ; Cc1cc(OC(F)(F)F)ccc1N1CN(c2cc(C)c(=O)n(COP(=O)(O)O)c2C)C(=O)c2cc(C(F)(F)F)cnc21 ; Cc1cc(F)ccc1N1CN(c2ccc(=O)n(COP(=O)(O)O)c2C)C(=O)c2cc(F)c(Cl)cc21 ; Cc1c(N2CN(c3ccc(=O)n(COP(=O)(O)O)c3C)C(=O)c3cc(F)c(Cl)cc32)ccc(F)c1F ; "and"]

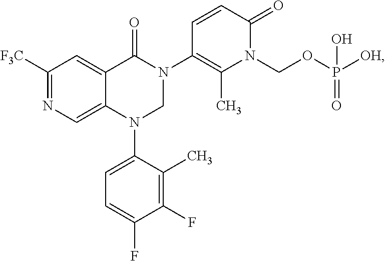 [SMILES: Cc1c(N2CN(c3ccc(=O)n(COP(=O)(O)O)c3C)C(=O)c3cc(C(F)(F)F)ncc32)ccc(F)c1F]

or a pharmaceutically acceptable salt thereof

Such compounds of the invention are prodrugs of their respective parent compounds, which are Naᵥ1.8 inhibitor compounds. The term “prodrug” refers to compounds that are drug precursors which, following administration and/or absorption, release the parent compound in vivo via a metabolic process. Typically, a prodrug has less biological activity than the parent compound. A prodrug may also improve the physical properties and/or efficacy of the parent compound, such as reduced toxicity and fewer unwanted effects through greater control of the absorption, blood levels, metabolic distribution and/or cellular uptake of the parent compound. Prodrugs may also have higher solubility than the corresponding parent compound.

Upon administration of the prodrug to a subject, such as a human, the prodrug moiety is cleaved thereby resulting in the parent compound. The terms “parent compound” and “parent drug” are used interchangeably herein and refer to the biologically active entity that is released via enzymatic action of a metabolic or catabolic process, or via a chemical process following administration of the prodrug. The parent compound may also be the starting material for the preparation of the corresponding prodrug. Without wishing to be bound by any theories, Naᵥ1.8 inhibitory activity upon administration of the prodrug is primarily due to formation of the parent compound from cleavage of the prodrug.

The prodrugs of the invention typically have higher aqueous solubility than the corresponding parent compounds. This higher solubility facilitates administration of higher doses of the prodrug, resulting in a greater drug load per unit dosage. Thus, the prodrug compounds of the invention may be advantageous for intravenous (IV) formulation and administration, and thus beneficial for use in the treatment of pain and pain associated diseases in which administration of higher doses or administration via the IV route may be beneficial, such as treatment of acute pain.

In one embodiment, provided is a compound which is:

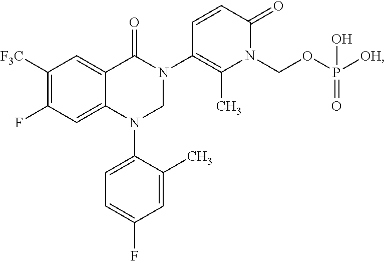 [SMILES: Cc1cc(F)ccc1N1CN(c2ccc(=O)n(COP(=O)(O)O)c2C)C(=O)c2cc(C(F)(F)F)c(F)cc21]

or a pharmaceutically acceptable salt thereof.

In one embodiment, provided is a compound which is:

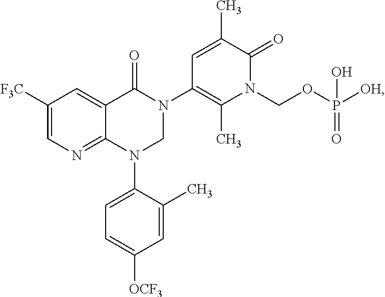 [SMILES: Cc1cc(OC(F)(F)F)ccc1N1CN(c2cc(C)c(=O)n(COP(=O)(O)O)c2C)C(=O)c2cc(C(F)(F)F)cnc21]

or a pharmaceutically acceptable salt thereof.

In one embodiment, provided is a compound which is:

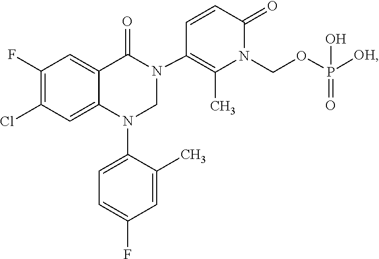 [SMILES: Cc1cc(F)ccc1N1CN(c2ccc(=O)n(COP(=O)(O)O)c2C)C(=O)c2cc(F)c(Cl)cc21]

or a pharmaceutically acceptable salt thereof.

In one embodiment, provided is a compound which is:

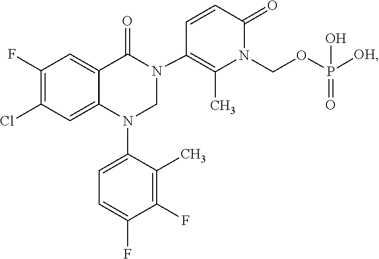 [SMILES: Cc1c(N2CN(c3ccc(=O)n(COP(=O)(O)O)c3C)C(=O)c3cc(F)c(Cl)cc32)ccc(F)c1F]

or a pharmaceutically acceptable salt thereof.

In one embodiment, provided is a compound which is:

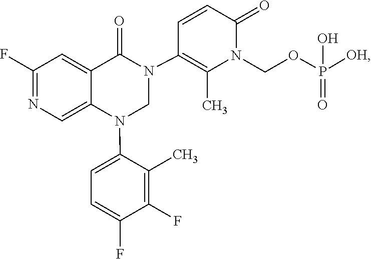 [SMILES: Cc1c(N2CN(c3ccc(=O)n(COP(=O)(O)O)c3C)C(=O)c3cc(F)ncc32)ccc(F)c1F]

or a pharmaceutically acceptable salt thereof.

In another aspect, provided is a compound selected from the group consisting of:

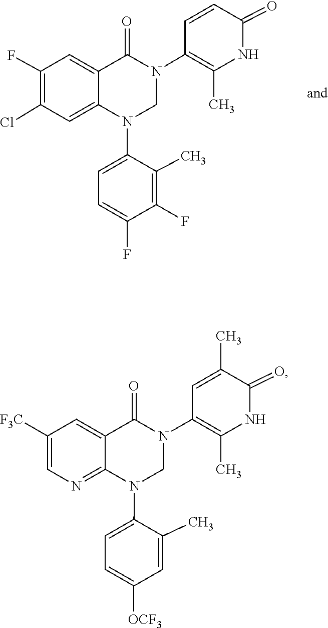 [SMILES: Cc1[nH]c(=O)ccc1N1CN(c2ccc(F)c(F)c2C)c2cc(Cl)c(F)cc2C1=O ; Cc1cc(OC(F)(F)F)ccc1N1CN(c2cc(C)c(=O)[nH]c2C)C(=O)c2cc(C(F)(F)F)cnc21]

or a tautomer thereof, or a pharmaceutically acceptable salt thereof such compounds of the invention are parent compounds of certain prodrugs of the invention, and have Naᵥ1.8 inhibitory activity.

In an embodiment, provided is a compound which is:

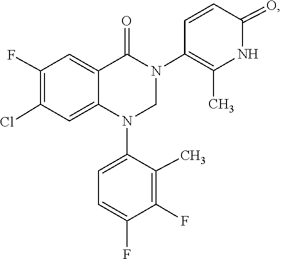 [SMILES: Cc1[nH]c(=O)ccc1N1CN(c2ccc(F)c(F)c2C)c2cc(Cl)c(F)cc2C1=O]

or a tautomer thereof, or a pharmaceutically acceptable salt thereof.

In an embodiment, provided is a compound which is:

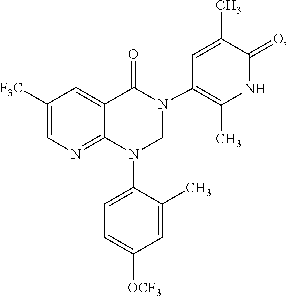 [SMILES: Cc1cc(OC(F)(F)F)ccc1N1CN(c2cc(C)c(=O)[nH]c2C)C(=O)c2cc(C(F)(F)F)cnc21]

or a tautomer thereof, or a pharmaceutically acceptable salt thereof

Salts

It is to be understood that the references herein to a compound of the invention and/or corresponding tautomer forms thereof or a salt thereof includes the compound and/or corresponding tautomer forms thereof as a free base or acid, or as a salt thereof, for example as a pharmaceutically acceptable salt thereof. Thus, in one embodiment, the invention is directed to a compound of the invention and/or corresponding tautomer forms thereof. In another embodiment, the invention is directed to a salt of a compound of the invention and/or corresponding tautomer forms thereof. In a further embodiment, the invention is directed to a pharmaceutically acceptable salt of a compound of the invention and/or corresponding tautomer forms thereof. In another embodiment, the invention is directed to a compound of the invention and/or corresponding tautomer forms thereof, or a salt thereof. In another embodiment, the invention is directed to a compound of the invention and/or corresponding tautomer forms thereof, or a pharmaceutically acceptable salt thereof.

Because of its potential use in medicine, it will be appreciated that a salt of a compound of the invention and/or corresponding tautomer forms thereof is preferably pharmaceutically acceptable.

The term “pharmaceutically acceptable” refers to those compounds (including salts), materials, compositions, and dosage forms which are, within the scope of sound medical judgment, suitable for use in contact with the tissues of human beings and animals without excessive toxicity, irritation, or other problem or complication, commensurate with a reasonable benefit/risk ratio.

The term “pharmaceutically acceptable salts” refers to salts that retain the desired biological activity of the subject compound and exhibit minimal undesired toxicological effects. These pharmaceutically acceptable salts may be prepared in situ during the final isolation and purification of the compound, or by separately reacting the purified compound in its free acid or free base form with a suitable base or acid, respectively. Furthermore, pharmaceutically acceptable salts of a compound of the invention and/or corresponding tautomer forms thereof may be prepared during further processing of the free acid or base form, for example in situ during manufacture into a pharmaceutical formulation.

Pharmaceutically acceptable salts include, amongst others, those described in Berge, J. Pharm. Sci., 1977, 66, 1-19, or those listed in P H Stahl and C G Wermuth, editors, Handbook of Pharmaceutical Salts; Properties, Selection and Use, Second Edition Stahl/Wermuth: Wiley-VCH/VHCA, 2011.

Non-pharmaceutically acceptable salts may be used, for example as intermediates in the preparation of a compound of the invention and/or corresponding tautomer forms thereof or a pharmaceutically acceptable salt thereof.

Suitable pharmaceutically acceptable salts can include acid or base addition salts. Such base addition salts can be formed by reaction of a compound of the invention and/or corresponding tautomer forms thereof (which, for example, contains an acidic functional group) with the appropriate base, optionally in a suitable solvent such as an organic solvent, to give the salt which can be isolated by a variety of methods, including crystallisation and filtration. Such acid addition salts can be formed by reaction of a compound of the invention and/or corresponding tautomer forms thereof (which, for example contains a basic amine or other basic functional group) with the appropriate acid, optionally in a suitable solvent such as an organic solvent, to give the salt which can be isolated by a variety of methods, including crystallization and filtration.

Salts may be prepared in situ during the final isolation and purification of a compound of the invention and/or corresponding tautomer forms thereof. If a basic compound of the invention and/or corresponding tautomer forms thereof is isolated as a salt, the corresponding free base form of that compound may be prepared by any suitable method known to the art, including treatment of the salt with an inorganic or organic base. Similarly, if a compound of the invention and/or corresponding tautomer forms thereof containing an acidic functional group is isolated as a salt, the corresponding free acid form of that compound may be prepared by any suitable method known to the art, including treatment of the salt with an inorganic or organic acid.

It will be understood that if a compound of the invention and/or corresponding tautomer forms thereof contains two or more basic moieties, the stoichiometry of salt formation may include 1, 2 or more equivalents of acid. Such salts would contain 1, 2 or more acid counterions, for example, a dihydrochloride salt.

Stoichiometric and non-stoichiometric forms of a pharmaceutically acceptable salt of a compound of the invention and/or corresponding tautomer forms thereof are included within the scope of the invention, including sub-stoichiometric salts, for example where a counterion contains more than one acidic proton.

Representative pharmaceutically acceptable acid addition salts include, but are not limited to, 4-acetamidobenzoate, acetate, adipate, alginate, ascorbate, aspartate, benzenesulfonate (besylate), benzoate, bisulfate, bitartrate, butyrate, calcium edetate, camphorate, camphorsulfonate (camsylate), caprate (decanoate), caproate (hexanoate), caprylate (octanoate), cinnamate, citrate, cyclamate, digluconate, 2,5-dihydroxybenzoate, disuccinate, dodecylsulfate (estolate), edetate (ethylenediaminetetraacetate), estolate (lauryl sulfate), ethane-1,2-disulfonate (edisylate), ethanesulfonate (esylate), formate, fumarate, galactarate (mucate), gentisate (2,5-dihydroxybenzoate), glucoheptonate (gluceptate), gluconate, glucuronate, glutamate, glutarate, glycerophosphorate, glycolate, hexylresorcinate, hippurate, hydrabamine (N,N′-di(dehydroabietyl)-ethylenediamine), hydrobromide, hydrochloride, hydroiodide, hydroxynaphthoate, isobutyrate, lactate, lactobionate, laurate, malate, maleate, malonate, mandelate, methanesulfonate (mesylate), methylsulfate, mucate, naphthalene-1,5-disulfonate (napadisylate), naphthalene-2-sulfonate (napsylate), nicotinate, nitrate, oleate, palmitate, p-aminobenzenesulfonate, p-aminosalicyclate, pamoate (embonate), pantothenate, pectinate, persulfate, phenylacetate, phenylethylbarbiturate, phosphate, polygalacturonate, propionate, p-toluenesulfonate (tosylate), pyroglutamate, pyruvate, salicylate, sebacate, stearate, subacetate, succinate, sulfamate, sulfate, tannate, tartrate, teoclate (8-chlorotheophyllinate), thiocyanate, triethiodide, undecanoate, undecylenate, and valerate.

Representative pharmaceutically acceptable base addition salts include, but are not limited to, aluminium, 2-amino-2-(hydroxymethyl)-1,3-propanediol (TRIS, tromethamine), arginine, benethamine (N-benzylphenethylamine), benzathine (N,N′-dibenzylethylenediamine), bis-(2-hydroxyethyl)amine, bismuth, calcium, chloroprocaine, choline, clemizole (1-p chlorobenzyl-2-pyrrolidine-1′-ylmethylbenzimidazole), cyclohexylamine, dibenzylethylenediamine, diethylamine, diethyltriamine, dimethylamine, dimethylethanolamine, dopamine, ethanolamine, ethylenediamine, L-histidine, iron, isoquinoline, lepidine, lithium, lysine, magnesium, meglumine (N-methylglucamine), piperazine, piperidine, potassium, procaine, quinine, quinoline, sodium, strontium, t-butylamine, and zinc.

In some embodiments, a compound is a pharmaceutically acceptable salt of a compound selected from the group consisting of:

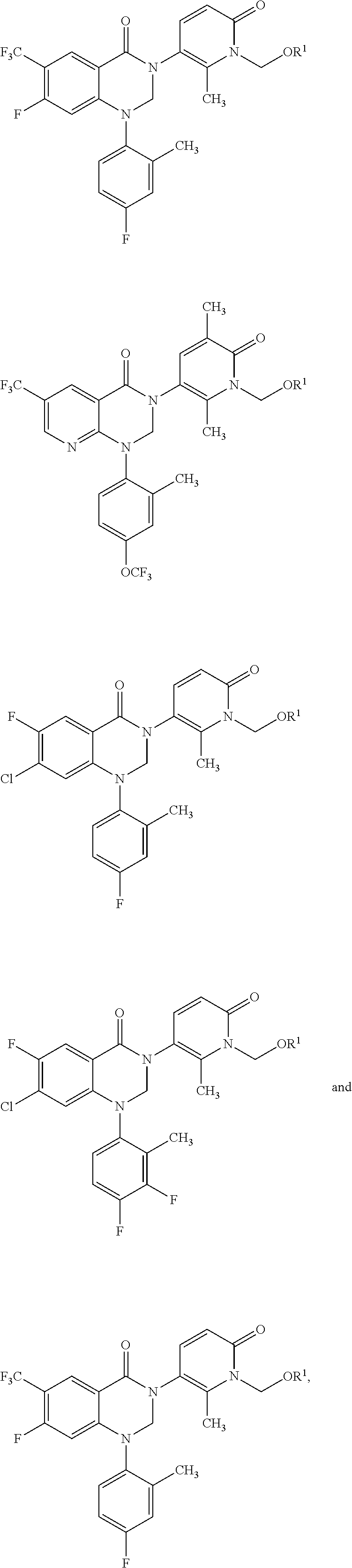

wherein:
   R¹ is —P(O)(OH)O⁻M⁺, —PO(O⁻)₂·2M⁺, or —PO(O⁻)₂·D²⁺;
   each M⁺ is independently a pharmaceutically acceptable monovalent cation; and
   D²⁺ is a pharmaceutically acceptable divalent cation.

Monovalent cations (M⁺) suitable for use in the invention include, but are not limited to, alkali metal ions and ammonium ions. As used herein, the term “alkali metal” refers to the Group I elements, which include, but are not limited to lithium (Li), sodium (Na), potassium (K) and the like. When two M⁺ are present, each M⁺ is independently a monovalent cation, wherein each M⁺ is the same or different. In some embodiments, when two M⁺ are present, each M⁺ is the same.

Divalent cations (D²⁺) suitable for use in the invention include, but are not limited to, alkaline earth metal ions and divalent aluminum ions. As used herein, the term “alkaline earth metal” refers to the Group II elements, which include, but are not limited to calcium (Ca), magnesium (Mg), strontium (Sr) and the like.

Other monovalent and divalent cations suitable for use in the invention include monovalent or divalent ions of amino acid ions, such as monovalent or divalent ions of arginine, lysine, ornithine, etc. Monovalent and divalent cations including basic nitrogen-containing groups can be prepared by quaternization with agents such as lower alkyl halides (e.g., methyl, ethyl, propyl, and butyl chlorides, bromides, and iodides), dialkyl sulfates (e.g., dimethyl, diethyl, dibutyl, and diamyl sulfates), etc.

In an embodiment, each M⁺ is independently an alkali metal ion. In another embodiment, each M⁺ is independently Li⁺, Na⁺, or K⁺. In another embodiment, each M⁺ is Li⁺. In another embodiment, each M⁺ is Na⁺. In another embodiment, each M⁺ is K⁺.

In an embodiment, each M⁺ is independently an ammonium ion. In another embodiment, each M⁺ is independently an ammonium ion of the formula —N(R^{a})₄, wherein each R^{a} is independently hydrogen, cyclohexyl, or —(C₁-C₆)alkyl optionally substituted with 1 to 6-OH groups. In another embodiment, each M⁺ is independently an ammonium ion selected from NH₄⁺, ethanolamine ion

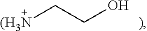

N-methyl-D-glucamine ion, and dicyclohexylamine ion. In another embodiment, each M⁺ is NH₄⁺. In another embodiment, each M⁺ is

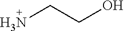

(ethanolamine ion).

In an embodiment, D²⁺ is an alkaline earth metal ion. In another embodiment, D²⁺ is Mg²⁺, Ca²⁺, or Sr₂⁺. In another embodiment, D²⁺ is Mg²⁺ or Ca²⁺. In another embodiment, D²⁺ is Mg²⁺. In another embodiment, D²⁺ is Ca².

In an embodiment, provided is a compound which is:

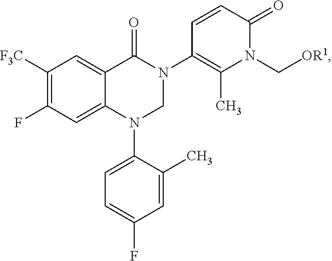

wherein:
R¹ is —P(O)(OH)O⁻M⁺, —PO(O⁻)₂·2M⁺, or —PO(O⁻)₂·D²;
each M⁺ is independently a pharmaceutically acceptable monovalent cation; and
D²⁺ is a pharmaceutically acceptable divalent cation.

In an embodiment, provided is a compound which is:

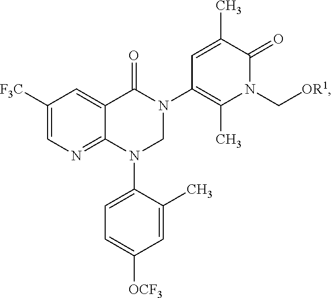

wherein:
R¹ is —P(O)(OH)O⁻M⁺, —PO(O⁻)₂·2M⁺, or —PO(O⁻)₂·D²⁺;
each M⁺ is independently a pharmaceutically acceptable monovalent cation; and
D²⁺ is a pharmaceutically acceptable divalent cation.

In an embodiment, provided is a compound which is:

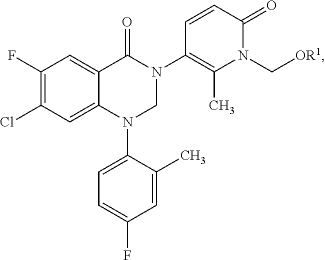

wherein:
R¹ is —P(O)(OH)O⁻M⁺, —PO(O⁻)₂·2M⁺, or —PO(O⁻)₂·D²⁺;
each M⁺ is independently a pharmaceutically acceptable monovalent cation; and
D²⁺ is a pharmaceutically acceptable divalent cation.

In an embodiment, provided is a compound which is:

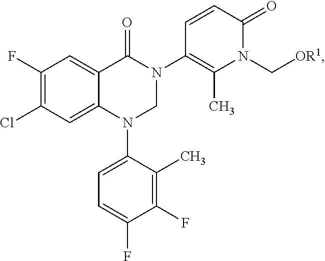

wherein:
R¹ is —P(O)(OH)O⁻M⁺, —PO(O⁻)₂·2M⁺, or —PO(O⁻)₂·D²⁺;
each M⁺ is independently a pharmaceutically acceptable monovalent cation; and
D²⁺ is a pharmaceutically acceptable divalent cation.

In an embodiment, provided is a compound which is:

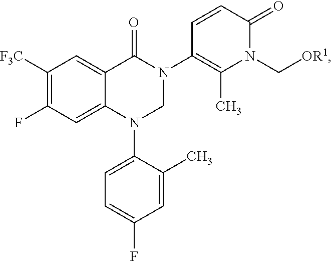

wherein:
R¹ is —P(O)(OH)O⁻M⁺, —PO(O⁻)₂·2M⁺, or —PO(O⁻)₂·D²⁺;
each M⁺ is independently a pharmaceutically acceptable monovalent cation; and
D²⁺ is a pharmaceutically acceptable divalent cation.

Solvates/Crystals/Co-Crystals

It will be appreciated that many organic compounds can form complexes with solvents in which they are reacted or from which they are precipitated or crystallized. These complexes are known as “solvates.” For example, a complex with water is known as a “hydrate.” Solvents with high boiling points and/or solvents with a high propensity to form hydrogen bonds such as water, ethanol, iso-propyl alcohol, and N-methyl pyrrolidinone may be used to form solvates. Methods for the identification of solvates include, but are not limited to, NMR and microanalysis. Compounds of the invention and/or corresponding tautomer forms thereof or salts thereof, may exist in solvated and unsolvated form.

The compounds of the invention may be in crystalline or amorphous form. The most thermodynamically stable crystalline form of a compound of the invention is of particular interest.

Crystalline forms of compounds of the invention may be characterized and differentiated using a number of conventional analytical techniques, including, but not limited to, X-ray powder diffraction (XRPD), infrared spectroscopy (IR), Raman spectroscopy, differential scanning calorimetry (DSC), thermogravimetric analysis (TGA) and solid-state nuclear magnetic resonance (ssNMR).

In one embodiment, the invention provides a crystalline form of (5-(7-chloro-1-(3,4-difluoro-2-methylphenyl)-6-fluoro-4-oxo-1,4-dihydroquinazolin-3(2H)-yl)-6-methyl-2-oxopyridin-1(2H)-yl)methyl dihydrogen phosphate.

In another embodiment, the invention provides a crystalline form of (5-(7-chloro-1-(3,4-difluoro-2-methylphenyl)-6-fluoro-4-oxo-1,4-dihydroquinazolin-3(2H)-yl)-6-methyl-2-oxopyridin-1(2H)-yl)methyl dihydrogen phosphate characterized by an X-ray powder diffraction (XRPD) pattern comprising diffraction angles, when measured using Cu K_{α} radiation, of about 11.9, about 13.2, about 14.7 and/or about 16.0 degrees 2θ.

In another embodiment, the invention provides a crystalline form of (5-(7-chloro-1-(3,4-difluoro-2-methylphenyl)-6-fluoro-4-oxo-1,4-dihydroquinazolin-3(2H)-yl)-6-methyl-2-oxopyridin-1(2H)-yl)methyl dihydrogen phosphate characterized by an X-ray powder diffraction (XRPD) pattern comprising diffraction angles, when measured using Cu K_{α} radiation, substantially as set out in Table 2.

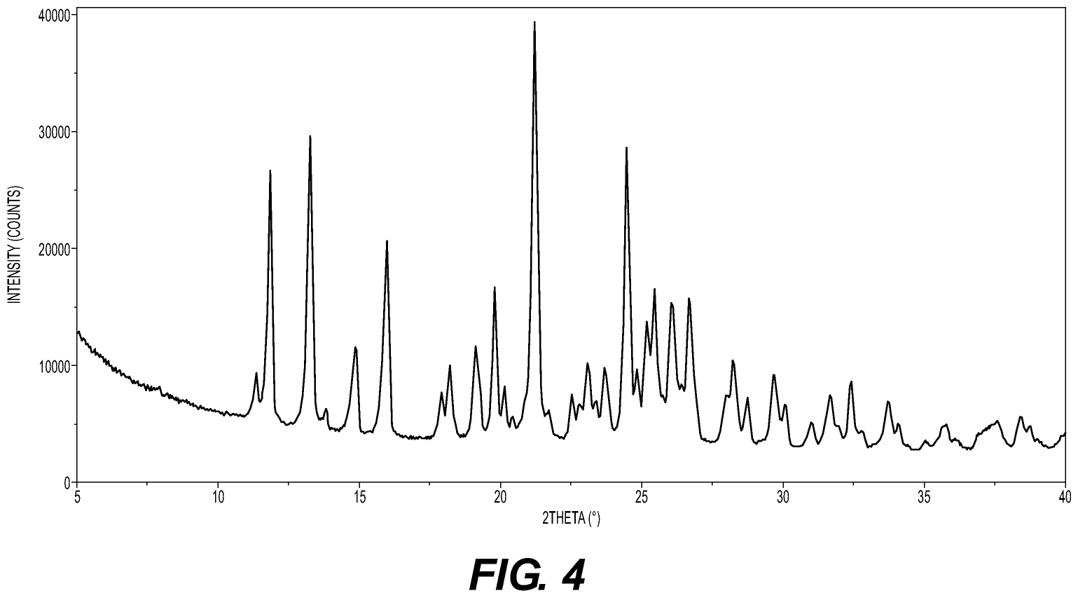

In a further embodiment, the invention provides a crystalline form of (5-(7-chloro-1-(3,4-difluoro-2-methylphenyl)-6-fluoro-4-oxo-1,4-dihydroquinazolin-3(2H)-yl)-6-methyl-2-oxopyridin-1(2H)-yl)methyl dihydrogen phosphate characterized by an X-ray powder diffraction (XRPD) pattern substantially in accordance with FIG. 4.

When it is indicated herein that there is a peak in an XRPD pattern at a given value, it is typically meant that the peak is within ±0.2, for example ±0.1, of the value quoted.

Stereoisomers

Compounds of the invention and/or corresponding tautomer forms thereof and pharmaceutically acceptable salts thereof may contain one or more asymmetric centers (also referred to as a chiral center) and may, therefore, exist as individual enantiomers, diastereomers, or other stereoisomeric forms, or as mixtures thereof. Chiral centers, such as chiral carbon atoms, may also be present in a substituent such as an alkyl group. Where the stereochemistry of a chiral center present in a compound of the invention or in any chemical structure illustrated herein, is not specified the structure is intended to encompass all individual stereoisomers and all mixtures thereof. Thus, compounds of the invention and/or corresponding tautomer forms thereof and pharmaceutically acceptable salts thereof containing one or more chiral centers may be used as racemic mixtures, enantiomerically enriched mixtures, or as enantiomerically pure individual stereoisomers.

Individual stereoisomers of a compound of the invention and/or corresponding tautomer forms thereof or a pharmaceutically acceptable salt thereof, which contain one or more asymmetric centers may be resolved by methods known to those skilled in the art. For example, such resolution may be carried out (1) by formation of diastereoisomeric salts, complexes or other derivatives; (2) by selective reaction with a stereoisomer-specific reagent, for example by enzymatic oxidation or reduction; or (3) by gas-liquid or liquid chromatography in a chiral environment, for example, on a chiral support such as silica with a bound chiral ligand or in the presence of a chiral solvent. The skilled artisan will appreciate that where the desired stereoisomer is converted into another chemical entity by one of the separation procedures described above, a further step is required to liberate the desired form. Alternatively, specific stereoisomers may be synthesized by asymmetric synthesis using optically active reagents, substrates, catalysts or solvents, or by converting one enantiomer to the other by asymmetric transformation.

Isotopes

The invention also includes all suitable isotopic variations of a compound described herein and/or corresponding tautomer forms thereof or a pharmaceutically acceptable salt thereof Δn isotopic variation of a compound of the invention and/or corresponding tautomer forms thereof or a pharmaceutically acceptable salt thereof, is defined as one in which at least one atom is replaced by an atom having the same atomic number but an atomic mass different from the atomic mass usually found in nature. Examples of isotopes that can be incorporated into compounds of the invention include isotopes of hydrogen, carbon, nitrogen, oxygen, fluorine and chlorine such as ²H, ³H, ¹³C, ¹⁴C, ¹⁵N, ¹⁷O, ¹⁸O, ¹⁸F and ³⁶Cl, respectively. Certain isotopic variations of a compound of the invention and/or corresponding tautomer forms thereof or a salt or solvate thereof, for example, those in which a radioactive isotope such as ³H or ¹⁴C is incorporated, are useful in drug and/or substrate tissue distribution studies. Tritiated, i.e., ³H, and carbon-14, i.e., ¹⁴C, isotopes are particularly preferred for their ease of preparation and detectability. Further, substitution with isotopes such as deuterium, i.e., ²H, may afford certain therapeutic advantages resulting from greater metabolic stability, for example, increased in vivo half-life or reduced dosage requirements and hence may be preferred in some circumstances. Isotopic variations of a compound of the invention and/or corresponding tautomer forms thereof or a pharmaceutically salt thereof, can generally be prepared by conventional procedures such as by the illustrative methods or by the preparations described in the Examples hereafter using appropriate isotopic variations of suitable reagents.

Tautomers

Moreover, compounds of the invention may exist as tautomers or in tautomeric forms. It is to be understood that any reference to a named compound or structurally depicted compound is intended to encompass all tautomers of such compound. It is conventionally understood in the chemical arts that tautomers are structural or constitutional isomers of chemical compounds that readily interconvert. This reaction commonly results in the relocation of a proton. A structural isomer, or constitutional isomer (per IUPAC), is a type of isomer in which molecules with the same molecular formula have different bonding patterns and atomic organization, as opposed to stereoisomers, in which molecular bonds are always in the same order and only spatial arrangement differs. The concept of tautomerizations is called tautomerism. The chemical reaction interconverting the two is called tautomerization. Care should be taken not to confuse tautomers with depictions of ‘contributing structures’ in chemical resonance. Tautomers are distinct chemical species and can be identified as such by their differing spectroscopic data, whereas resonance structures are merely convenient depictions and do not physically exist. For example, the 2-pyridone ring exhibits tautomerism, wherein the proton attached to the nitrogen can move to the oxygen to give the tautomeric form 2-hydroxypyridine:

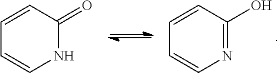 [SMILES: O=c1cccc[nH]1 ; Oc1ccccn1]

Pharmaceutical Compositions

In another aspect, the invention relates to a pharmaceutical composition comprising a compound of the invention or a tautomer thereof, or a pharmaceutically acceptable salt thereof, according to any one of the embodiments disclosed herein, and a pharmaceutically acceptable excipient (also referred to as carriers and/or diluents in the pharmaceutical arts). The excipients are acceptable in the sense of being compatible with the other ingredients of the formulation and not deleterious to the recipient thereof (i.e., the patient).

A pharmaceutically acceptable excipient is non-toxic and should not interfere with the efficacy of the active ingredient. Suitable pharmaceutically acceptable excipients will vary depending upon the particular dosage form chosen, route of administration, etc. Suitable pharmaceutically acceptable excipients include the following types of excipients: diluents, carriers, fillers, binders, disintegrants, lubricants, glidants, granulating agents, coating agents, wetting agents, solvents, co-solvents, suspending agents, emulsifiers, sweeteners, flavoring agents, flavor masking agents, coloring agents, anti-caking agents, humectants, chelating agents, plasticizers, viscosity increasing agents, antioxidants, preservatives, stabilizers, surfactants, and buffering agents. Examples of pharmaceutically acceptable excipients are described, e.g., in REMINGTON'S PHARMACEUTICAL SCIENCES (Mack Publishing Company), THE HANDBOOK OF PHARMACEUTICAL ADDITIVES (Gower Publishing Limited), and THE HANDBOOK OF PHARMACEUTICAL EXCIPIENTS (the American Pharmaceutical Association and the Pharmaceutical Press).

Pharmaceutical compositions may be adapted for administration by any appropriate or suitable route, for example by systemic administration (e.g., oral administration, parenteral administration, transdermal administration, rectal administration, inhalation), topical administration, etc. Parenteral administration is typically by injection or infusion and includes intravenous, intramuscular, and subcutaneous injection or infusion. Inhalation refers to administration into the patient's lungs whether inhaled through the mouth or through the nasal passages. Typically, administration is via the oral route or parenteral route, such as intravenous route.

Pharmaceutical compositions adapted for oral administration may be presented as solid dosage forms such as tablets, capsules, caplets, troches, pills; powders; or liquid dosage forms such as solutions, suspensions, syrups, elixirs, or emulsion, etc. Pharmaceutical compositions adapted for parenteral administration may be presented as solutions, suspensions, and powders for reconstitution.

In some embodiments, a pharmaceutical composition of the invention is formulated for oral administration. In other embodiments, a pharmaceutical composition of the invention is formulated for intravenous administration.

In general, pharmaceutical compositions of the invention are prepared using conventional materials and techniques, such as mixing, blending and the like. Some of the methods commonly used in the art are described in Remington's PHARMACEUTICAL SCIENCES (Mack Publishing Company).

Solid oral dosage forms, such as tablets and capsules can be prepared by mixing a compound of the invention with excipients such as diluents and fillers (e.g., starch, lactose, sucrose, calcium carbonate, calcium phosphate and the like), binders (e.g., starch, acacia gum, carboxymethyl cellulose, hydroxypropyl cellulose, crystalline cellulose, and the like), lubricants (e.g., magnesium stearate, talc and the like), and the like. Pharmaceutical compositions adapted for parenteral administration can be an injection solution prepared from powders, granules or tablets by mixing with a carrier, such as distilled water, saline and the like, and base and the like may be used for pH adjustment.

The invention also provides a pharmaceutical composition comprising from 0.5 to 1,000 mg of a compound of the invention and from 0.5 to 1,000 mg of a pharmaceutically acceptable excipient.

Compounds and pharmaceutical compositions of the invention as defined herein may be administered once or according to a dosing regimen, where a number of doses are administered at varying intervals of time for a given period of time. For example, doses may be administered one, two, three, or four times per day. Doses may be administered until the desired therapeutic effect is achieved or indefinitely to maintain the desired therapeutic effect. Doses of compounds of the invention may be in the range of 0.001 mg/kg to 100 mg/kg, such as 0.001 mg/kg to 50 mg/kg. Preferably, the selected dose is administered orally or parenterally.

In accordance with another aspect of the invention there is provided a process for the preparation of a pharmaceutical composition comprising mixing (or admixing) a compound of the invention or a tautomer thereof or salt thereof (e.g., pharmaceutically acceptable salt thereof) with at least one pharmaceutically acceptable excipient.

Synthetic Schemes and General Methods of Preparation

The invention also relates to processes for preparing compounds of the invention disclosed herein. The compounds of the invention may be made by any number of processes using conventional organic syntheses as described in the Schemes below and more specifically illustrated by the exemplary compounds which follow in the Examples section herein, or by drawing on the knowledge of a skilled organic chemist. Suitable synthetic routes are depicted below in the following general reaction schemes. The synthesis procedures provided in the following Schemes are applicable for producing compounds of the invention disclosed herein, having a variety of different functional groups as defined employing appropriate precursors.

Those skilled in the art will appreciate that in the preparation of compounds of the invention, it may be necessary and/or desirable to protect one or more sensitive groups in the molecule or the appropriate intermediate to prevent undesirable side reactions. The skilled artisan will appreciate that if a substituent described herein is not compatible with the synthetic methods described herein, the substituent may be protected with a suitable protecting group that is stable to the reaction conditions. The protecting group may be removed at a suitable point in the reaction sequence to provide a desired intermediate or target compound. Suitable protecting groups for use according to the present invention are well-known to those skilled in the art and may be used in a conventional manner. See for example, “Protective Groups in Organic Synthesis” by T. W. Green and P. G. M Wets (Wiley & Sons, 1991) or “Protecting Groups” by P. J. Kocienski (Georg Thieme Verlag, 1994). Subsequent deprotection, where needed, affords compounds of the nature generally disclosed. In some instances, a substituent may be specifically selected to be reactive under the reaction conditions used. Under these circumstances, the reaction conditions convert the selected substituent into another substituent that is either useful as an intermediate compound or is a desired substituent in a target compound.

While the Schemes shown below are representative of methods for preparing compounds of the invention, they are only intended to be illustrative of processes that may be used to make the compounds of the invention. Intermediates (compounds used in the preparation of the compounds of the invention) also may be present as salts. Thus, in reference to intermediates, the phrase “compound(s) of formula (number)” means a compound having that structural formula or a pharmaceutically acceptable salt thereof. Compound names were generated using the software naming program ChemDraw 5 Ultra v16.0, available from Perkin Elmer, 940 Winter Street, Waltham, Massachusetts, 02451, USA.

Several methods for preparing the compounds of this invention are illustrated in the following Schemes and Examples. Starting materials are either commercially available or made by known procedures in the literature or as illustrated.

General Synthetic Schemes

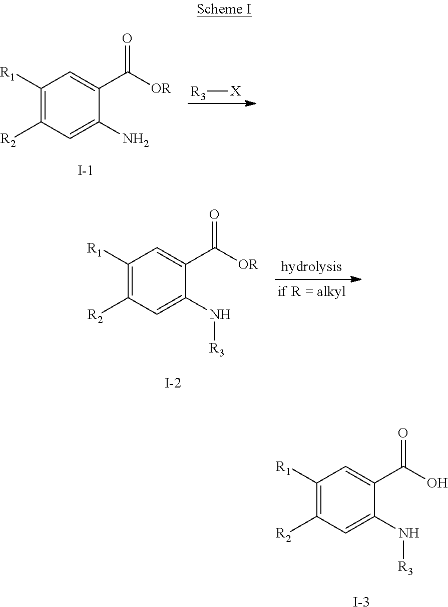

The preparation of the compounds of the invention typically begins with the synthesis of N-substituted-2-aminoaromatic derivatives I-3 (Scheme I). Arylation of the aniline nitrogen with an appropriate aryl halide can be accomplished using a transition metal catalyst such as Pd₂(dba)₃ or Cu/CuO, a suitable ligand, such as BINAP or Xantphos and an inorganic base, such as Cs₂CO₃ or K₂CO₃ in an appropriate solvent such as 1,4-dioxane. In some cases where X=F, the conversion may be achieved via an SNAr reaction in the presence of a base, such as diisopropylethylamine (DIPEA) in an appropriate solvent like dimethylformamide (DMF).

In each of intermediates I-1, I-2, and I-3 in Scheme I, each of R₁ and R₂ is independently halo (e.g., —F or —Cl) or haloalkyl- (e.g., —CF₃); R₃, where present, is optionally substituted phenyl; and R is H or alkyl.

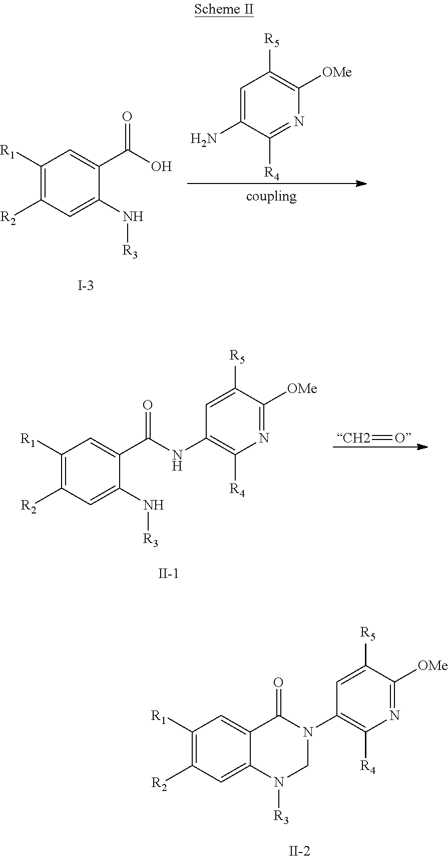

The intermediate N-substituted-2-aminoaromatic acid derivatives I-3, prepared as illustrated in Scheme I, can be converted to II-1 by coupling of I-3 with a suitable aryl-NH₂, for example 2-methoxy-4-aminopyridine, under various amide couple conditions known to those of skill in the art, provides the corresponding amide TI-1 (Scheme II). For example, one might employ standard coupling reagents, like EDC/HOBT, HATU, HBTU or T3P, in the presence of an amine base, like triethylamine, or Hunig's base (diisopropylethylamine), in a suitable solvent, typically DMF, DMA or acetonitrile. Alternatively, one might convert the acid to the corresponding acid chloride, using a reagent like thionyl chloride or oxalyl chloride, then react the acid chloride with a suitable aryl-NH₂ (like 2-methoxy-4-aminopyridine), in the presence of an acid scavenger or base, such as pyridine, 2,6-lutidine, triethylamine or Hunig's base, in an appropriate solvent, such as dichloromethane or pyridine, to afford the desired coupling product II-1. Formation of the dihydroquinazolinone ring system, as in II-2, involves reaction of II-1 with formaldehyde or a suitable equivalent. For instance, the reaction may be achieved using formaldehyde, either as gaseous formaldehyde, paraformaldehyde, or s-trioxane, in the presence of an acid, preferably PTSA or sulfuric acid. Alternatively, the dihydroquinazolinone ring system can be formed via reaction of II-1 using diiodomethane or chloroiodomethane as a formaldehyde equivalent. In this variant of the cyclization reaction, a base, typically Cs₂CO₃ or NaH, could be used, in a suitable solvent, oftentimes acetonitrile or DMF. The choice of using formaldehyde or diiodomethane depends on the particular reactivity characteristics of the substrate II-1.

In each of intermediates I-3, II-1, and II-2 in Scheme II, each of R₁ And R₂ is independently halo (e.g., —F or —Cl) or haloalkyl- (e.g., —CF₃); R₃ is optionally substituted phenyl; and each of R₄ and R₅ is independently hydrogen or —CH₃.

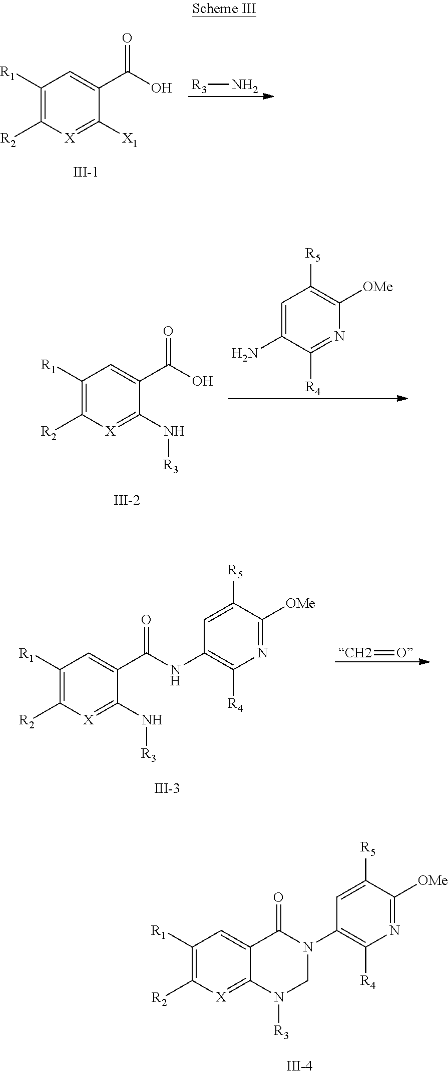

In a variation of the methods described in Scheme I and II, the compounds can be prepared as illustrated in Scheme III. For activated 2-halo acids (III-1; X₁=Cl, Br or I) reactions can be employed with a suitable aniline or amine nucleophile, to afford III-2, typically under elevated temperatures using either standard heating or microwave irradiation, in the presence of a catalyst, for example Pd₂(dba)₃ or Cu/CuO, a suitable ligand, for instance BINAP or Xantphos, and an inorganic base, typically Cs₂CO₃ or K₂CO₃, in an appropriate solvent, such as 1,4-dioxane, toluene or 2-ethoxyethanol. In some cases where X₁=F, the conversion may be achieved through a SNAr reaction in the presence of a base, for example DIPEA in an appropriate solvent like DMF. The conversion of III-2 to III-3 and ultimately III-4 can be accomplished as described in Scheme II.

In each of intermediates III-1, III-2, III-3, and III-4 in Scheme III, each of R₁ and R₂ is independently halo (e.g., —F or —Cl) or haloalkyl- (e.g., —CF₃); R₃, where present, is optionally substituted phenyl; each of R₄ and R₅ is independently hydrogen or —CH₃; and X is CH or N.

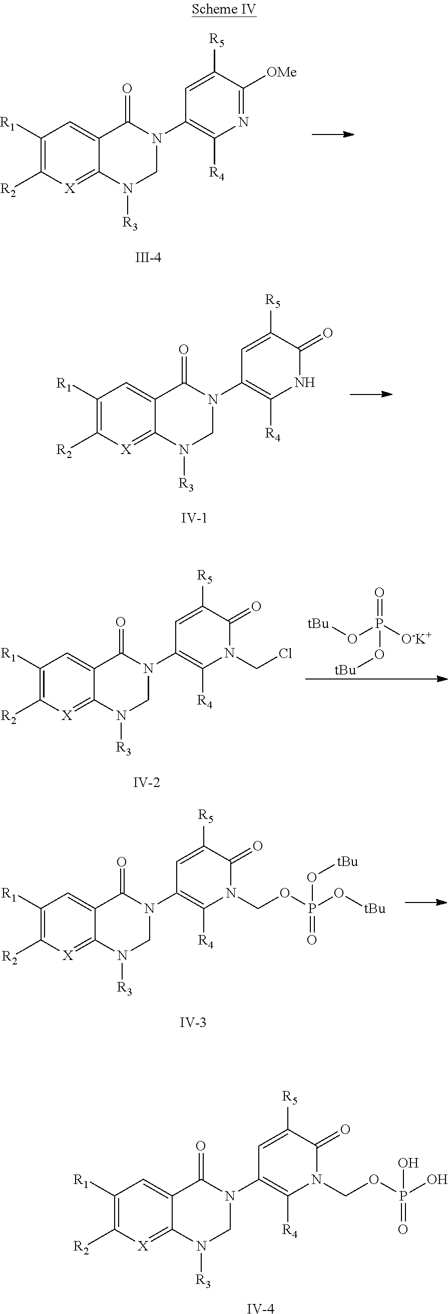

As shown in Scheme IV, transformation of compound III-4 to the pyridone IV-1 can be achieved by reacting compound III-4 with a mixture of TMS-chloride and NaI, or a solution of TMS-iodide, in a neutral solvent like acetonitrile, at elevated temperature. Compound IV-1 can be reacted with chloromethyl chloroformate in the presence of an organic base such as DABCO in suitable solvents such as EtOAc and DMF to provide the chloromethylpyridone IV-2. Reaction of compound IV-2 with potassium di-tert-butyl phosphate in the presence of a phase transfer catalyst such as TBAI in solvent DMF at elevated temperature provides compound IV-3. Removal of the tert-butyl protecting groups under acidic conditions such as acetic acid in acetonitrile and water provides the prodrug compounds of the invention.

In each of intermediates III-4, IV-1, IV-2, IV-3, and IV-4 in Scheme IV, each of R₁ and R₂ is independently halo (e.g., —F or —Cl) or haloalkyl- (e.g., —CF₃); R₃ is optionally substituted phenyl; each of R₄ and R₅ is independently hydrogen or —CH₃; and X is CH or N.

Methods/Uses

In general, the invention also relates to uses of the compounds and/or pharmaceutical compositions described herein for use as a medicament or for use in therapy.

Compounds of the invention as defined herein are inhibitors of voltage-gated sodium ion channels, and particularly the voltage-gated sodium ion channel Naᵥ1.8. The activity of a compound utilized in this invention as an inhibitor of Naᵥ1.8 can be assayed according to methods described generally in the Examples herein, or according to methods available to one of ordinary skill in the art.

In one aspect, the invention relates to uses of compounds and pharmaceutical compositions as described herein as inhibitors of voltage-gated sodium ion channels, particularly Naᵥ1.8.

In an embodiment, the invention relates to a method of inhibiting a voltage-gated sodium ion channel in a subject in need thereof, comprising administering to the subject an effective amount of a compound of the invention or a pharmaceutical composition of the invention as described herein. In another embodiment, the voltage-gated sodium channel is Naᵥ1.8.

In an embodiment, the invention relates to a compound of the invention or a pharmaceutical composition of the invention for use in inhibiting a voltage-gated sodium ion channel. In another embodiment, the voltage-gated sodium channel is Naᵥ1.8.

In an embodiment, the invention relates to use of a compound of the invention or a pharmaceutical composition of the invention in the manufacture of a medicament for inhibiting a voltage-gated sodium ion channel. In another embodiment, the voltage-gated sodium channel is Naᵥ1.8.

Without wishing to be bound by any particular theory, the compounds and compositions of the invention are particularly useful for treating a disease, condition, or disorder where activation or hyperactivity of Naᵥ1.8 is implicated in the disease, condition, or disorder. When activation or hyperactivity of Naᵥ1.8 is implicated in a particular disease, condition, or disorder, the disease, condition, or disorder may also be referred to as a “Naᵥ1.8-mediated disease, condition or disorder.” Exemplary Naᵥ1.8-mediated diseases, conditions, and disorders include pain and pain-associated diseases, and cardiovascular diseases, such as atrial fibrillation.

According to embodiments of the invention, a pain-associated disease is pain caused by any one of a variety of diseases of varying etiologies as described throughout the disclosure. In some embodiments, pain or a pain-associated disease is neuropathic pain, chronic pain, acute pain, nociceptive pain, inflammatory pain, musculoskeletal pain, visceral pain, cancer pain, idiopathic pain, multiple sclerosis, Charcot-Marie-Tooth syndrome, or incontinence.

In some embodiments, pain or a pain-associated disease is neuropathic pain or chronic neuropathic pain. In some embodiments, pain or a pain-associated disease is neuropathic pain or chronic neuropathic pain selected from small fiber neuropathy, small fiber-mediated diabetic neuropathy, idiopathic small fiber neuropathy, painful diabetic neuropathy or polyneuropathy.

In some embodiments pain or a pain-associated disease is neuropathic pain selected from post-herpetic neuralgia, diabetic neuralgia, painful HIV-associated sensory neuropathy, trigeminal neuralgia, burning mouth syndrome, post-amputation pain, phantom pain, painful neuroma, traumatic neuroma, Morton's neuroma, nerve entrapment injury, spinal stenosis, carpal tunnel syndrome, radicular pain, sciatica pain, nerve avulsion injury, brachial plexus avulsion, complex regional pain syndrome, drug therapy induced neuralgia, cancer chemotherapy induced neuralgia, anti-retroviral therapy induced neuralgia, post spinal cord injury pain, idiopathic small-fiber neuropathy, idiopathic sensory neuropathy or trigeminal autonomic cephalalgia.

In some embodiments, pain or a pain-associated disease is neuropathic pain or chronic neuropathic pain selected from diabetic peripheral neuropathy, pain caused by neuropathy, neurologic or neuronal injury, pain associated nerve injury, neuralgias and associated acute or chronic pain, post-herpetic neuralgia, pain associated root avulsions, painful traumatic mononeuropathy, painful polyneuropathy, erythromelalgia, paroxysmal extreme pain disorder (PEPD), burning mouth syndrome, central pain syndromes caused by a lesion at a level of nervous system, traumatic nerve injury, nerve compression or entrapment, congenital insensitivity to pain (CIP), dysmenorrheal, primary erythromelalgia, HIV peripheral sensory neuropathy, pudendal neuralgia, spinal nerve injury, chronic inflammatory demyelinating polyneuropathy (CIDP), carpal tunnel syndrome and vasculitic neuropathy.

In some embodiments, pain or a pain-associated disease is visceral pain, wherein visceral pain is inflammatory bowel disease pain, Crohn's disease pain or interstitial cystitis pain.

In some embodiments, pain or a pain-associated disease is musculoskeletal pain, wherein musculoskeletal pain is osteoarthritis pain, back pain, cold pain, burn pain or dental pain.

In some embodiments, pain or a pain-associated disease is idiopathic pain, wherein idiopathic pain is fibromyalgia pain.

In some embodiments, pain or a pain-associated disease is chronic or acute pre-operative associated pain or chronic or acute post-operative associated pain. Post-operative associated pain includes ambulatory post-operative pain. Ambulatory surgery, also known as outpatient surgery, refers to same day surgery that does not require an overnight stay in a hospital or other medical facility. In some embodiments, pre-operative associated pain is selected from neuropathic pain or chronic neuropathic pain, chronic osteoarthritis pain, dental pain or inflammatory pain. In some embodiments, post-operative associated pain is selected from bunionectomy pain, hernia repair pair, breast surgery pain or cosmetic surgical pain.

In some embodiments, pain or a pain-associated disease is pain caused by trauma or iatrogenic medical or dental procedures. As used herein, the term “iatrogenic” refers to pain induced inadvertently by a medical or dental personnel, such as surgeon or dentist, during medical or dental treatment(s) or diagnostic procedure(s), which include, but are not limited to pain caused by pre-operative (i.e., “before”), peri-operative (i.e., “during” or medically induced pain during non-surgical or operative treatment(s)) and post-operative (i.e., after, post-operative or surgical induced caused pain) medical or dental procedures.

In some embodiments, pain or a pain-associated disease is nociceptive pain, wherein nociceptive pain is post-surgical pain, cancer pain, back and craniofacial pain, osteoarthritis pain, dental pain or diabetic peripheral neuropathy.

In some embodiments, pain or a pain-associated disease is inflammatory pain. Inflammatory pain can be pain of varied physiological origins. In some embodiments, inflammatory pain is selected from pain associated with osteoarthritis, rheumatoid arthritis, rheumatic disorder, teno-synovitis and gout, shoulder tendonitis or bursitis, gouty arthritis, and polymyalgia rheumatica, primary hyperalgesia, secondary hyperalgesia, primary allodynia, secondary allodynia, or other pain caused by central sensitization, complex regional pain syndrome, chronic arthritic pain and related neuralgias or acute pain. In some embodiments inflammatory pain is selected from pain associated with rheumatoid arthritis, osteoarthritis, rheumatoid spondylitis, gouty arthritis or juvenile arthritis. In some embodiments, inflammatory pain is selected from rheumatoid arthritis, rheumatoid spondylitis, gouty arthritis, juvenile arthritis, rheumatic disorder, gout, shoulder tendonitis or bursitis, polymyalgia rheumatica, primary hyperalgesia, secondary hyperalgesia, primary allodynia, secondary allodynia, or other pain caused by central sensitization, complex regional pain syndrome, chronic or acute arthritic pain and related neuralgias. In some embodiments, inflammatory pain is rheumatoid arthritis pain or vulvodynia.

In some embodiments, inflammatory pain is osteoarthritis, chronic osteoarthritis pain (e.g., hip or knee) or chronic inflammatory demyelinating polyneuropathy.

In some embodiments pain or a pain-associated disease is musculoskeletal pain. In some embodiments, musculoskeletal pain is selected from bone and joint pain, osteoarthritis, lower back and neck pain, or pain resulting from physical trauma or amputation. In some embodiments, musculoskeletal pain is selected from bone and joint pain, osteoarthritis (e.g., knee, hip), tendonitis (e.g., shoulder), bursitis (e.g., shoulder) tenosynovitis, lower back and neck pain, sprains, strains, or pain resulting from physical trauma or amputation.

In some embodiments, pain or a pain-associated disease is neurologic or neuronal injury associated or related pain disorders caused by diseases selected from neuropathy, pain associated nerve injury, pain associated root avulsions, painful traumatic mononeuropathy, painful polyneuropathy, erythromelalgia, paroxysmal extreme pain disorder (PEPD), burning mouth syndrome; central pain syndromes caused by a lesion at a level of nervous system), traumatic nerve injury, nerve compression or entrapment, congenital insensitivity to pain (CIP), dysmenorrheal, primary erythromelalgia; HIV peripheral sensory neuropathy, pudendal neuralgia, spinal nerve injury, chronic inflammatory demyelinating polyneuropathy (CIDP), carpal tunnel syndrome or vasculitic neuropathy.

In some embodiments, pain or a pain-associated disease is pain caused by trauma, or pain caused by iatrogenic, medical, or dental procedures.

In some embodiments, pain or a pain-associated disease is myofascial pain, myositis or muscle inflammation, repetitive motion pain, complex regional pain syndrome, sympathetically maintained pain, cancer, toxins and chemotherapy related pain, postsurgical pain syndromes and/or associated phantom limb pain, post-operative medical or dental procedures or treatments pain, or pain associated with HIV or pain induced by HIV treatment.

In some embodiments, pain or a pain-associated disease, disorder, or condition is neuropathic pain or other pain-associated disease selected from peripheral neuropathic pain, central neuropathic pain, inherited erythromelalgia (IEM), small fiber neuralgia (SFN), paroxysmal extreme pain disorder (PEPD), painful diabetic neuropathy, chronic lower back pain, neuropathic back pain, sciatica, non-specific lower back pain, multiple sclerosis pain, HIV-related neuropathy, post-herpetic neuralgia, trigeminal neuralgia, vulvodynia, pain resulting from physical trauma, post-limb amputation pain, neuroma pain, phantom limb pain, cancer, toxins, or chronic inflammatory conditions.

In some embodiments, pain or a pain-associated disease is acute pain, chronic pain, neuropathic pain, inflammatory pain, arthritis, migraine, cluster headaches, trigeminal neuralgia, herpetic neuralgia, general neuralgias, epilepsy, epilepsy conditions, neurodegenerative disorders, psychiatric disorders, anxiety, depression, dipolar disorder, myotonia, arrhythmia, movement disorders, neuroendocrine disorders, ataxia, multiple sclerosis, irritable bowel syndrome, incontinence, visceral pain, osteoarthritis pain, postherpetic neuralgia, diabetic neuropathy, radicular pain, sciatica, back pain, head pain, neck pain, severe pain, intractable pain, nociceptive pain, breakthrough pain, postsurgical pain, cancer pain, stroke, cerebral ischemia, traumatic brain injury, amyotrophic lateral sclerosis, stress induced angina, exercise induced angina, palpitations, hypertension, or abnormal gastro-intestinal motility.

In some embodiments, pain or a pain-associated disease is femur cancer pain, non-malignant chronic bone pain, rheumatoid arthritis, osteoarthritis, spinal stenosis, neuropathic low back pain, myofascial pain syndrome, fibromyalgia, temporomandibular joint pain, chronic visceral pain, abdominal pain, pancreatic pain, IBS pain, chronic and acute headache pain, migraine, tension headache (including cluster headaches), chronic and acute neuropathic pain, post-herpetic neuralgia, diabetic neuropathy, HIV-associated neuropathy, trigeminal neuralgia, Charcot-Marie Tooth neuropathy, hereditary sensory neuropathies, peripheral nerve injury, painful neuromas, ectopic proximal and distal discharges, radiculopathy, chemotherapy induced neuropathic pain, radiotherapy-induced neuropathic pain, post-mastectomy pain, central pain, spinal cord injury pain, post-stroke pain, thalamic pain, complex regional pain syndrome, phantom pain, intractable pain, acute pain, acute post-operative pain, acute musculoskeletal pain, joint pain, mechanical low back pain, neck pain, tendonitis, injury/exercise pain, acute visceral pain, pyelonephritis, appendicitis, cholecystitis, intestinal obstruction, hernias, chest pain, cardiac pain, pelvic pain, renal colic pain, acute obstetric pain, labor pain, cesarean section pain, acute inflammatory, burn and trauma pain, acute intermittent pain, endometriosis, acute herpes zoster pain, sickle cell anemia, acute pancreatitis, breakthrough pain, orofacial pain including sinusitis pain, dental pain, multiple sclerosis (MS) pain, pain in depression, leprosy pain, Behcet's disease pain, adiposis dolorosa, phlebitic pain, Guillain-Barre pain, painful legs and moving toes, Haglund syndrome, erythromelalgia pain, Fabry's disease pain, bladder and urogenital disease, including, urinary incontinence, hyperactivity bladder, painful bladder syndrome, interstitial cyctitis (IC), prostatitis, complex regional pain syndrome (CRPS) (type I and type II), widespread pain, paroxysmal extreme pain, pruritis, tinnitis, or angina-induced pain.

In another aspect, the invention relates to uses of compounds and pharmaceutical compositions of the invention in methods and medicaments for treating cardiovascular diseases, including atrial fibrillation and cardiac arrhythmias.

In some embodiments, a cardiovascular disease is atrial fibrillation that is either idiopathic in nature or caused by a disease as defined herein. Atrial fibrillation can be paroxysmal atrial fibrillation, sustained atrial fibrillation, long-standing atrial fibrillation, atrial fibrillation with heart failure, atrial fibrillation with cardiac valve disease, or atrial fibrillation with chronic kidney disease. In particular embodiments, atrial fibrillation is selected from paroxysmal, sustained, or long-standing atrial fibrillation.

In some embodiments, a cardiovascular disease includes cardiac arrhythmias.

Thus, in another aspect, the invention also provides a method of treatment in a subject, especially a human. Disease states which can be treated by the methods and compositions provided herein include, but are not limited to, pain and pain associated diseases, and cardiovascular diseases.

The term “treatment” refers to alleviating the specified condition, eliminating or reducing one or more symptoms of the condition, slowing or eliminating the progression of the condition, and delaying the reoccurrence of the condition in a previously afflicted patient or subject.

As used herein, “effective amount” and “therapeutically effective amount” are used interchangeably. The term “therapeutically effective amount” refers to the quantity of a compound of the invention or a tautomer thereof, or a pharmaceutically acceptable salt thereof, which will elicit the desired biological response in the human body. It may vary depending on the compound, the disease and its severity, and the age and weight of the subject to be treated.

The term “subject” refers to a human body.

In one aspect, the invention relates to a method of treatment of pain or a pain-associated disease as defined herein in a human in need thereof, comprising administering to the human a compound of the invention or a pharmaceutical composition of the invention as described herein.

In an embodiment, provided is a method of treatment of acute pain or chronic pain in a human in need thereof, comprising administering to the human a compound of the invention or a pharmaceutical composition of the invention as described herein.

In an embodiment, provided is a method of treatment of pain caused by trauma, pain caused by iatrogenic medical or dental procedures, or pre-operative or post-operative associated pain in a human in need thereof, comprising administering to the human a compound of the invention or a pharmaceutical composition of the invention as described herein.

In an embodiment, provided is a method of treatment of neuropathic pain, nociceptive pain, inflammatory pain, musculoskeletal pain, visceral pain, or idiopathic pain in a human in need thereof, comprising administering to the human a compound of the invention or a pharmaceutical composition of the invention as described herein.

In an embodiment, provided is a method of treatment of neuropathic pain or chronic neuropathic pain selected from the group consisting of small fiber neuropathy, small fiber-mediated diabetic neuropathy, idiopathic small fiber neuropathy, painful diabetic neuropathy and polyneuropathy in a human in need thereof, comprising administering to the human a compound of the invention or a pharmaceutical composition of the invention as described herein.

In an embodiment, provided is a method of treatment of inflammatory pain selected from the group consisting of osteoarthritis, chronic osteoarthritis pain, and chronic inflammatory demyelinating polyneuropathy in a human in need thereof, comprising administering to the human a compound of the invention or a pharmaceutical composition of the invention as described herein.

In an embodiment, provided is a method of treatment of a pain or a pain-associated disease selected from the group consisting of neuropathic pain, ambulatory post-operative pain, and osteoarthritis in a human in need thereof, comprising administering to the human a compound of the invention or pharmaceutical composition of the invention as described herein. In some embodiments, the pain or pain-associated disease is neuropathic pain. In some embodiments, the pain or pain-associated disease is chronic neuropathic pain. In some embodiments, the pain or pain-associated disease is small fiber neuropathy. In some embodiments, the pain or pain-associated disease is ambulatory post-operative pain. In some embodiments, the pain or pain-associated disease is osteoarthritis. In some embodiments, the pain or pain-associated disease is osteoarthritis of the knee and/or osteoarthritis of the hip.

In another aspect, the invention provides compounds of the invention and pharmaceutical compositions of the invention as described herein for use in treatment of pain or a pain-associated disease as defined herein.

In an embodiment, provided is a compound of the invention or pharmaceutical composition of the invention for use in treatment of acute pain or chronic pain.

In an embodiment, provided is a compound of the invention or pharmaceutical composition of the invention for use in treatment of pain caused by trauma, pain caused by iatrogenic medical or dental procedures, or pre-operative or post-operative associated pain.

In an embodiment, provided is a compound of the invention or pharmaceutical composition of the invention for use in treatment of neuropathic pain, nociceptive pain, inflammatory pain, musculoskeletal pain, visceral pain, or idiopathic pain.

In an embodiment, provided is a compound of the invention or pharmaceutical composition of the invention for use in treatment of neuropathic pain or chronic neuropathic pain selected from the group consisting of small fiber neuropathy, small fiber-mediated diabetic neuropathy, idiopathic small fiber neuropathy, painful diabetic neuropathy and polyneuropathy.

In an embodiment, provided is a compound of the invention or pharmaceutical composition of the invention for use in treatment of inflammatory pain selected from the group consisting of osteoarthritis, chronic osteoarthritis pain, and chronic inflammatory demyelinating polyneuropathy.

In an embodiment, provided is a compound of the invention or pharmaceutical composition of the invention for use in treatment of pain or a pain-associated disease selected from the group consisting of neuropathic pain, ambulatory post-operative pain, and osteoarthritis. In some embodiments, the pain or pain-associated disease is neuropathic pain.

In some embodiments, the pain or pain-associated disease is chronic neuropathic pain. In some embodiments, the pain or pain-associated disease is small fiber neuropathy. In some embodiments, the pain or pain-associated disease is ambulatory post-operative pain. In some embodiments, the pain or pain-associated disease is osteoarthritis. In some embodiments, the pain or pain-associated disease is osteoarthritis of the knee and/or osteoarthritis of the hip.

In another aspect, the invention also provides uses of compounds of the invention or pharmaceutical compositions of the invention as described herein in the manufacture of a medicament for treatment of pain and pain associated diseases as described herein.

In an embodiment, provided is use of a compound of the invention or pharmaceutical composition of the invention in the manufacture of a medicament for treatment of acute pain or chronic pain.

In an embodiment, provided is use of a compound of the invention or pharmaceutical composition of the invention in the manufacture of a medicament for treatment of pain caused by trauma, pain caused by iatrogenic medical or dental procedures, or pre-operative or post-operative associated pain.

In an embodiment, provided is use of a compound of the invention or pharmaceutical composition of the invention in the manufacture of a medicament for treatment of neuropathic pain, nociceptive pain, inflammatory pain, musculoskeletal pain, visceral pain, or idiopathic pain.

In an embodiment, provided is use of a compound of the invention or pharmaceutical composition of the invention in the manufacture of a medicament for treatment of neuropathic pain or chronic neuropathic pain selected from the group consisting of small fiber neuropathy, small fiber-mediated diabetic neuropathy, idiopathic small fiber neuropathy, painful diabetic neuropathy and polyneuropathy.

In an embodiment, provided is use of a compound of the invention or pharmaceutical composition of the invention in the manufacture of a medicament for treatment of inflammatory pain selected from the group consisting of osteoarthritis, chronic osteoarthritis pain, and chronic inflammatory demyelinating polyneuropathy.

In an embodiment, provided is use of a compound of the invention or pharmaceutical composition of the invention in the manufacture of a medicament for treatment of pain or a pain-associated disease selected from the group consisting of neuropathic pain, ambulatory post-operative pain, and osteoarthritis. In some embodiments, the pain or pain-associated disease is neuropathic pain. In some embodiments, the pain or pain-associated disease is chronic neuropathic pain. In some embodiments, the pain or pain-associated disease is small fiber neuropathy. In some embodiments, the pain or pain-associated disease is ambulatory post-operative pain. In some embodiments, the pain or pain-associated disease is osteoarthritis. In some embodiments, the pain or pain-associated disease is osteoarthritis of the knee and/or osteoarthritis of the hip.

In one aspect, the invention relates to a method of treatment of atrial fibrillation as defined herein in a human in need thereof, comprising administering to the human a compound of the invention or a pharmaceutical composition of the invention as described herein. In some embodiments, the atrial fibrillation is selected from the group consisting of paroxysmal atrial fibrillation, sustained atrial fibrillation, long-standing atrial fibrillation, atrial fibrillation with heart failure, atrial fibrillation with cardiac valve disease, and atrial fibrillation with chronic kidney disease.

In another aspect, the invention relates to a compound of the invention or a pharmaceutical composition of the invention for use in treatment of atrial fibrillation. In some embodiments, the atrial fibrillation is selected from the group consisting of paroxysmal atrial fibrillation, sustained atrial fibrillation, long-standing atrial fibrillation, atrial fibrillation with heart failure, atrial fibrillation with cardiac valve disease, and atrial fibrillation with chronic kidney disease.

In another aspect, the invention relates to use of a compound of the invention or a pharmaceutical composition of the invention as described herein in the manufacture of a medicament for treatment of atrial fibrillation. In some embodiments, the atrial fibrillation is selected from the group consisting of paroxysmal atrial fibrillation, sustained atrial fibrillation, long-standing atrial fibrillation, atrial fibrillation with heart failure, atrial fibrillation with cardiac valve disease, and atrial fibrillation with chronic kidney disease.

In another aspect, the invention relates to a compound of the invention or a pharmaceutical composition of the invention as described herein for use in therapy.

Combination Therapy

The compounds and pharmaceutical compositions of the invention disclosed herein can be combined with or co-administered with other therapeutic agents, particularly agents that may enhance the activity or time of disposition of the compounds. Combination therapies according to the invention comprise the administration of at least one compound of the invention and the use of at least one other treatment method, including administration of one or more other therapeutic agents.

By the term “co-administration” and derivatives thereof as used herein refers to either simultaneous administration or any manner of separate sequential administration of a Naᵥ1.8 inhibiting compound of the invention, as described herein, and an additional active ingredient. An additional active ingredient includes any compound or therapeutic agent known to or that demonstrates advantageous properties when administered to a human in need of treatment. Typically, if the administration is not simultaneous, the compounds are administered in a close time proximity to each other. Furthermore, the compounds may be administered in the same or separate dosage form, e.g., one compound may be administered orally and another compound may be administered intravenously.

Other therapeutic agents which may be used in combination with a compound of the invention include, but are not limited to Acetaminophen, Acetylsalicylic acid, Nav1.7 Inhibitors, Nav1.9 Inhibitors, anti-depressants (i.e. such as, but not limited to duloxetine or amitriptyline), anti-convulsants (i.e. such as, but not limited to pregabalin and gabapentin), opiates (i.e., such as, but not limited to hydrocodone, codeine, morphine, oxycodone, oxymorphone, fentanyl, and the like), etc.; and where administration of the above, respectively, also is determined by one of ordinary skill in the art. In one aspect, suitable Nav1.7 Inhibitors or Nav1.9 Inhibitors for use in the invention, include, but are not limited to those Nav1.7 Inhibitors or Nav1.9 Inhibitors known in the chemical literature.

Each component of a combination used for therapeutic purposes (e.g., compound or pharmaceutical composition of the invention and additional therapeutic agent) may be administered orally, intravenously or parenterally or in combinations thereof. Each component of a therapeutic combination may be, but is not limited to being administered by simultaneous administration, co-administration, or serial administration; and/or by identical or different routes of administration or combinations of administration routes. In certain embodiments, each identical or different route of administration or combinations of administration routes is selected from oral, intravenous or parenteral administration.

### EXAMPLES

The following examples illustrate the invention. These examples are not intended to limit the scope of the invention, but rather to provide guidance to the skilled artisan to prepare and use the compounds, compositions, and methods of the invention. While particular aspects or embodiments of the invention are described, the skilled artisan will appreciate that various changes and modifications can be made without departing from the spirit and scope of the invention.

### Synthesis Examples

It will be understood by the skilled artisan that purification methods (using acidic or basic modifiers) or compound workup procedures (using acidic or basic conditions) may result in formation of a salt of a title compound (for example, hydrobromic acid, formic acid, hydrochloric acid, trifluoroacetic acid, or ammonia salts of a title compound). The invention is intended to encompass such salts.

Final compounds were characterized with GCMS and LCMS (conditions listed below) and NMR. ¹H NMR or ¹⁹FNMR spectra were recorded using a Bruker Avance III 500 MHz spectrometer, Bruker Avance 400 MHz spectrometer and Varian Mercury Plus-300 MHz 10 spectrometer. CDCl₃ is deuteriochloroform, DMSO-d₆ is hexadeuteriodimethylsulfoxide, and CD₃OD is tetradeuteriomethanol. Chemical shifts are reported in parts per million (ppm) downfield from the internal standard tetramethylsilane (TMS) or the NMR solvent. Abbreviations for NMR data are as follows: s=singlet, d=doublet, t=triplet, q=quartet, m=multiplet, dd=doublet of doublets, dt=doublet of triplets, app=apparent, br=broad. J 15 indicates the NMR coupling constant measured in Hertz.

HPLC Methods:

Method A: UPLC: Waters Acquity equipped with an Acquity CSH, C₁₈ (2.1 mm×30 mm, 1.7 μm column) using a gradient of 1-100% MeCN/H₂O/0.1% TFA over 1.85 min at 1.3 m/min flow rate. Mass determinations were conducted using an Agilent 6110 Quadrupole MS with positive ESI.

Method B: UPLC: Waters Acquity equipped with an Acquity CSH, C₁₈ (2.1 mm×30 mm, 1.7 μm column) using a gradient of 1-100% MeCN/H₂O/0.1% 10 mM ammonium Bicarbonate in water adjusted to pH 10 with 25% ammonium hydroxide solution, over 1.85 min at 1.3 mL/min flow rate. Mass determinations were conducted using an Agilent 6110 Quadrupole MS with positive ESI.

In the following experimental descriptions, the following abbreviations may be used:

| Abbreviation | Meaning |
|---|---|
| AcOH | acetic acid |
| aq. | aqueous |
| BBr₃ | boron tribromide |
| BCl₃ | boron trichloride |
| BH₃ | borane |
| BINAP | 2,2′-bis(diphenylphosphino)-1,1′-binaphthalene |
| Bn | benzyl |
| brine | saturated aqueous sodium chloride |
| BuLi or nBuLi | butyllithium |
| CDI | carbonyldiimidazole |
| CH₂Cl₂ | methylene chloride |
| CH₃CN | acetonitrile |
| COCl₂ | oxalyl chloride |
| Cs₂CO₃ | cesium carbonate |
| DABCO | 1,4-diazabicyclo[2.2.2]octane |
| DCC | dicyclohexylcarbodiimide |
| DCM or | methylene chloride |
| CH₂Cl₂ | |
| DEAD | diethyl azodicarboxylate |
| DEAP | diethyl aminopyridine |
| DIAD | diisopropyl azodicarboxylate |
| DIPEA, DIEA, | N,N-diisopropylethylamine |
| Hunig's base | |
| DMA | Dimethylacetamide |
| DMAP | 4-dimethylaminopyridine |
| DMF | N,N-dimethylformamide |
| DME | dimethoxyethane |
| DMSO | dimethylsulfoxide |
| EDC | 1-[3-(dimethylamino)propyl]-3-ethylcarbodiimide |
| | hydrochloride |
| Et | ethyl |
| Et₃N | triethylamine |
| Et₂O | diethyl ether |
| EtOAc | ethyl acetate |
| EtOH | ethanol |
| Fmoc or fmoc | fluorenylmethyloxycarbonyl |
| g, G, gm, GM | gram |
| GCMS | gas chromatography-mass spectroscopy |
| h or hr | hour(s) |
| H₂ | hydrogen |
| HCl | Hydrochloric acid |
| H₂O₂ | hydrogen peroxide |
| H₂O | water |
| H₂SO₄ | sulfuric acid |
| HATU | (O-(7-azabenzotriazol-1-yl)-N,N,N′,N′- |
| | tetramethyluronium hexafluorophosphate) |
| HBTU | 2-(1H-benzo[d][1,2,3]triazol-1-yl)-1,1,3,3- |
| | tetramethylisouronium hexafluorophosphate(V) |
| HCl | hydrochloric acid |
| HCO₂H | formic acid |
| HOBt or HOBT | 1-hydroxy benzotriazole |
| HPLC | high performance liquid chromatography |
| Hz | Hertz |
| I₂ | Iodine |
| IPA | Isopropyl alcohol |
| JLR | jacketed lab reactor |
| K₂CO₃ | potassium carbonate |
| KHSO₄ | potassium hydrogen sulfate |
| KOAc | potassium acetate |
| L or l | liter |
| LAH | lithium aluminum hydride |
| LCMS | liquid chromatography-mass spectroscopy |
| LDA | lithium diisopropyl amide |
| LED | light-emitting diode |
| LiOH | lithium hydroxide |
| LHMDS | lithium bis(trimethylsilyl)amide |
| mCPBA | meta-chloroperoxybenzoic acid |
| MDAP | mass directed autopurification |
| Me | methyl |
| MeOH | methanol |
| mg, MG | milligram |
| MgBr₂ | magnesium bromide |
| MgSO₄ | magnesium sulfate |
| Mhz | megahertz |
| Min or mins | minute(s) |
| ml or mL or | milliliter |
| ML | |
| mmol | millimole |
| MnO₂ | manganese dioxide |
| Mol, mol | mole |
| MS | mass spectrum |
| MTBE | methyl tert-butyl ether |
| μw | microwave |
| N₂ | nitrogen |
| Na(CN)BH₃ | sodium cyanoborohydride |
| NaCl | sodium chloride |
| Na₂CO₃ | sodium carbonate |
| NaHCO₃ | sodium bicarbonate |
| NaHMDS | sodium bis(trimethylsilyl)amide |
| NaHSO₃ | sodium bisulfite |
| NaH | sodium hydride |
| NaI | sodium iodide |
| NaOH | sodium hydroxide |
| Na₂SO₃ | sodium sulfite |
| Na₂SO₄ | sodium sulfate |
| NH₄Cl | ammonium chloride |
| HCO₂•NH₄ | ammonium formate |
| NH₄OH | ammonium hydroxide |
| nm | nanometer |
| NMO | 4-methylmorpholine N-oxide |
| NMP | N-methyl-2-pyrrolidone |
| NMR | Nuclear magnetic resonance |
| Pd/C | palladium on carbon |
| PdCl₂(dbpf) | 1,1′-bis(di-tert-butylphosphino)ferrocene |
| | dichloropalladium |
| Pd(dppf)Cl₂/ | [1,1′-bis(diphenylphosphino)ferrocene] |
| PdCl₂(dppf) | dichloropalladium(II) |
| PdCl₂(dppf)— | [1,1′-bis(diphenylphosphino)ferrocene] |
| CH₂Cl₂ adduct | dichloropalladium(II), complex with dichloromethane |
| Pd₂(dba)₃ | tris(dibenzylideneacetone)dipalladium(0) |
| Pd(Ph₃)₄, | tetrakis(triphenylphosphine)palladium(0) |
| tetrakis | |
| PdOAc₂ or | palladium acetate |
| Pd(OAc)₂ | |
| Pd(OH)₂ | palladium hydroxide |
| PIFA | [Bis(trifluoroacetoxy)iodo]benzene |
| Ph | phenyl |
| PL HCO₃ MP | macroporus polystyrene supported carbonate |
| POCl₃ | phosphoryl chloride |
| psi | Pounds per square inch |
| PTFE | polytetrafluoroethylene |
| PTSOH or | p-Toluenesulfonic acid |
| PTSA or | |
| pTsOH | |
| rt or RT | room temperature |
| sat. | saturated |
| SFC | supercritical fluid chromatography |
| Si | silica |
| Si SPE | silica gel cartridges |
| SiO₂ | silica gel |
| SPE | solid phase extraction |
| T3P ® | propylphosphonic anhydride |
| tBu or t-Bu | tert-butyl group |
| TBAB | tetrabutylammonium bromide |
| TBAF | tetrabutylammonium fluoride |
| TBAI | tetrabutylammonium iodide |
| TBDMSCl | tert-butyldimethylsilyl chloride |
| TBME | tert-butylmethyl ether |
| TBTU | 2-(1H-benzotriazole-1-yl)-1,1,3,3-tetramethyluronium |
| | tetrafluoroborate |
| TEA | triethylamine |
| TFA | trifluoroacetic acid |
| THF | tetrahydrofuran |
| TiCl₄ | titanium tetrachloride |
| TMS—Br or | trimethylsilyl bromide |
| TMSBr | |
| TMS—Cl or | trimethylsilyl chloride |
| TMSCl | |
| TMSI | Iodotrimethylsilane or trimethylsilyl iodide |
| TMS—OTf | trimethylsilyl triflate |
| or TMSOtf | |
| tR | retention time |
| UPLC | ultra performance liquid chromatography |
| Xantphos | 4,5-Bis(diphenylphosphino)-9,9-dimethylxanthene |
| Xphos | 2-Dicyclohexylphosphino-2′,4′,6′-triisopropylbiphenyl |

### Example 1: (5-(7-fluoro-1-(4-fluoro-2-methylphenyl)-4-oxo-6-(trifluoromethyl)-1,4-dihydroquinazolin-3(2H)-yl)-6-methyl-2-oxopyridin-1(2H)-yl)methyl dihydrogen phosphate

### Step A: 3-(1-(chloromethyl)-2-methyl-6-oxo-1,6-dihydropyridin-3-yl)-7-fluoro-1-(4-fluoro-2-methylphenyl)-6-(trifluoromethyl-2,3-dihydroquinazolin-41H)-one

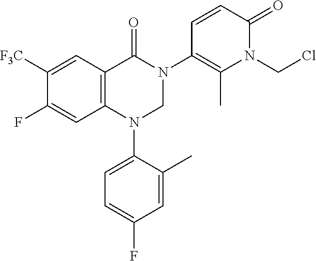 [SMILES: Cc1cc(F)ccc1N1CN(c2ccc(=O)n(CCl)c2C)C(=O)c2cc(C(F)(F)F)c(F)cc21]

To a solution of 7-fluoro-1-(4-fluoro-2-methylphenyl)-3-(2-methyl-6-oxo-1,6-dihydropyridin-3-yl)-6-(trifluoromethyl)-2,3-dihydroquinazolin-4(1H)-one (which may be synthesized as described in Example 107 of WO2020/261114) (6.73 g, 14.98 mmol) and DABCO (2.52 g, 22.46 mmol) in ethyl acetate (140.0 ml) and N,N-dimethylformamide (DMF) (14.00 ml), was added chloromethyl chloroformate (4.00 ml, 44.9 mmol) dropwise, when a white precipitate formed right away. The reaction mixture was heated at 85° C. for 18 hours. Additional chloromethyl chloroformate (2.66 ml, 30.0 mmol) was added and the reaction was heated for additional 3 hours. Additional DABCO (0.420 g, 3.74 mmol) and chloromethyl chloroformate (2.66 ml, 30.0 mmol) was again added and the reaction was heated for additional 19 hours. Finally, chloromethyl chloroformate (1.332 ml, 14.98 mmol) was added and the reaction was heated for 4 hours. The reaction mixture was cooled, diluted with more EtOAc and quenched with water and stirred for 10 minutes. The solid was filtered and washed with water to afford 3.90 g of desired product as a light yellow solid. The layers of the filtrate were separated and the aqueous layer was extracted with EtOAc (2×). The combined organics were washed with sat. aq NaHCO₃, brine and dried over Na₂SO₄ and concentrated. The residue was triturated with DCM to afford a light brown solid, which was further triturated with water to afford additional 1.8 g of desired product as an off white solid. The two solids were combined to afford 3-(1-(chloromethyl)-2-methyl-6-oxo-1,6-dihydropyridin-3-yl)-7-fluoro-1-(4-fluoro-2-methylphenyl)-6-(trifluoromethyl)-2,3-dihydroquinazolin-4(1H)-one (5.71 g, 11.01 mmol, 73.5% yield) as an off white solid. HPLC/MS 1.24 min (Method B), [M+H]⁺ 498.0. ¹H NMR (DMSO-d₆, 400 MHz) δ 8.10 (d, 1H, J=7.8 Hz), 7.4-7.6 (m, 2H), 7.3-7.4 (m, 1H), 7.2-7.2 (m, 1H), 6.4-6.5 (m, 1H), 5.6-6.4 (m, 3H), 4.8-5.6 (m, 2H), 2.4-2.5 (m, 3H), 2.2-2.3 (m, 3H).

### Step B: di-tert-butyl ((5-(7-fluoro-1-(4-fluoro-2-methylphenyl)-4-oxo-6-(trifluoromethyl)-1,4-dihydroquinazolin-3(2H)-yl)-6-methyl-2-oxopyridin-1(2H)-yl)methyl) phosphate

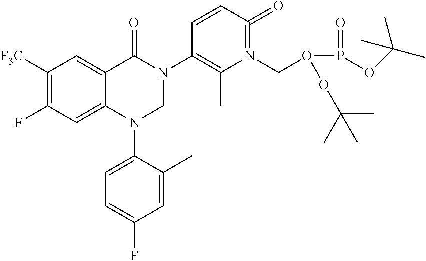

To a solution of 3-(1-(chloromethyl)-2-methyl-6-oxo-1,6-dihydropyridin-3-yl)-7-fluoro-1-(4-fluoro-2-methylphenyl)-6-(trifluoromethyl)-2,3-dihydroquinazolin-4(1H)-one (6.20 g, 12.45 mmol) and potassium di-tert-butyl phosphate (4.64 g, 18.68 mmol) in N,N-dimethylformamide (DMF) (170 ml) was added tetrabutylammonium iodide (0.460 g, 1.245 mmol). The reaction mixture was heated at 70° C. for 3 hours when a gel formed. The reaction mixture was cooled and quenched with water when a precipitate formed. The solid was filtered and washed with water to afford di-tert-butyl ((5-(7-fluoro-1-(4-fluoro-2-methylphenyl)-4-oxo-6-(trifluoromethyl)-1,4-dihydroquinazolin-3(2H)-yl)-6-methyl-2-oxopyridin-1(2H)-yl)methyl) phosphate (7.24 g, 10.24 mmol, 82% yield) as a light yellow solid. HPLC/MS 1.36 min (Method B), [M+H]⁺ 672.2. ¹H NMR (DMSO-d₆, 400 MHz) δ 8.10 (d, 1H, J=8.3 Hz), 7.4-7.5 (m, 2H), 7.3-7.4 (m, 1H), 7.2-7.3 (m, 1H), 6.4-6.4 (m, 1H), 6.14 (dd, 1H, J=12.7, 18.1 Hz), 5.7-5.9 (m, 2H), 4.7-5.6 (m, 2H), 2.3-2.4 (m, 3H), 2.2-2.3 (m, 3H), 1.41 (s, 18H).

### Step C: (5-(7-fluoro-1-(4-fluoro-2-methylphenyl)-4-oxo-6-(trifluoromethyl)-1,4-dihydroquinazolin-3(2H)-yl)-6-methyl-2-oxopyridin-1(2H)-yl)methyl dihydrogen phosphate

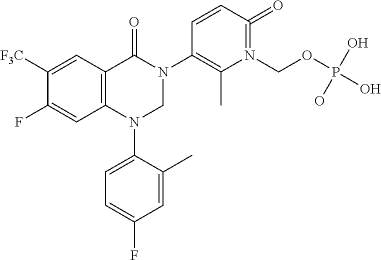

A slurry of di-tert-butyl ((5-(7-fluoro-1-(4-fluoro-2-methylphenyl)-4-oxo-6-(trifluoromethyl)-1,4-dihydroquinazolin-3(2H)-yl)-6-methyl-2-oxopyridin-1(2H)-yl)methyl) phosphate (7.20 g, 10.18 mmol) in acetone (70 mL) and water (56.0 mL) was heated at 50° C. for 6 hours (solution cleared). The reaction mixture was brought to room temperature and stirred for additional 20 hours. The volatiles were evaporated in vacuo at 35° C. and the aqueous portion was freeze dried to afford an off white solid, which was triturated with DCM:ether (1:3) to afford (5-(7-fluoro-1-(4-fluoro-2-methylphenyl)-4-oxo-6-(trifluoromethyl)-1,4-dihydroquinazolin-3(2H)-yl)-6-methyl-2-oxopyridin-1(2H)-yl)methyl dihydrogen phosphate (5.10 g, 8.66 mmol, 85% yield) as an off white solid. HPLC/MS 0.78 min (Method B), [M+H]⁺ 560.2. ¹H NMR (METHANOL-d₄, 400 MHz) δ 8.25 (d, 1H, J=7.8 Hz), 7.5-7.6 (m, 1H), 7.4-7.4 (m, 1H), 7.2-7.3 (m, 1H), 7.1-7.2 (m, 1H), 6.53 (d, 1H, J=9.8 Hz), 6.1-6.2 (m, 1H), 5.99 (d, 2H, J=6.8 Hz), 4.8-5.6 (m, 2H), 2.5-2.6 (m, 3H), 2.3-2.4 (m, 3H); ¹H NMR (DMSO-d₆, 400 MHz) δ 8.10 (d, 1H, J=8.3 Hz), 7.4-7.5 (m, 2H), 7.3-7.4 (m, 1H), 7.2-7.3 (m, 1H), 6.4-6.4 (m, 1H), 6.14 (dd, 1H, J=13.0, 18.3 Hz), 5.7-5.8 (m, 2H), 4.7-5.6 (m, 2H), 2.3-2.4 (m, 3H), 2.2-2.3 (m, 3H) (two OH protons hidden).

### Example 2: (3,6-dimethyl-5-(1-(2-methyl-4-(trifluoromethoxy)phenyl)-4-oxo-6-(trifluoromethyl)-1,4-dihydropyrido[2,3-d]pyrimidin-3(2H)-yl)-2-oxopyridin-1(2H)-yl)methyl dihydrogen phosphate

### Step A: 2-((2-methyl-4-(trifluoromethoxy)phenyl)amino)-5-(trifluoromethyl)nicotinic acid

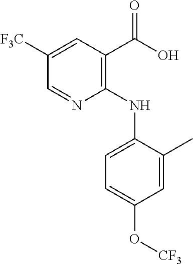 [SMILES: Cc1cc(OC(F)(F)F)ccc1Nc1ncc(C(F)(F)F)cc1C(=O)O]

To a mixture containing 2-methyl-4-(trifluoromethoxy)aniline (4.24 g, 22.17 mmol) in water (50 ml) was added 2-chloro-5-(trifluoromethyl)nicotinic acid (5 g, 22.17 mmol), p-TsOH (1.265 g, 6.65 mmol) and pyridine (1.793 ml, 22.17 mmol). The reaction mixture was heated at 95° C. for 4 days. The reaction mixture was cooled to room temperature, then diluted with 150 mL water and stirred for 1 hour. The solid was filtered and washed with water (3×25 mL), dried under vacuum to afford 2-((2-methyl-4-(trifluoromethoxy)phenyl)amino)-5-(trifluoromethyl)nicotinic acid (6.886 g, 17.20 mmol, 78% yield) as an orange-yellow solid. HPLC/MS 0.93 min (Method B), [M+H]⁺ 381.0. ¹H NMR (DMSO-d₆, 400 MHz) δ 14.16 (br s, 1H), 10.55 (s, 1H), 8.69 (dd, 1H, J=1.0, 2.4 Hz), 8.42 (d, 1H, J=2.4 Hz), 8.15 (d, 1H, J=9.3 Hz), 7.32 (s, 1H), 7.24 (dd, 1H, J=2.2, 9.0 Hz), 2.32 (s, 3H).

### Step B: N-(6-methoxy-2,5-dimethylpyridin-3-yl)-2-((2-methyl-4-(trifluoromethoxy)phenyl)amino)-5-(trifluoromethyl)nicotinamide

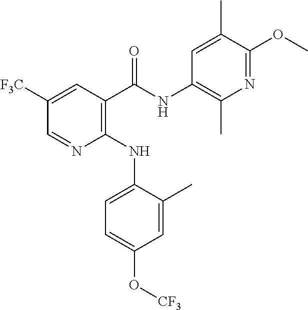 [SMILES: COc1nc(C)c(NC(=O)c2cc(C(F)(F)F)cnc2Nc2ccc(OC(F)(F)F)cc2C)cc1C]

To a solution of 2-((2-methyl-4-(trifluoromethoxy)phenyl)amino)-5-(trifluoromethyl)nicotinic acid (4.95 g, 12.37 mmol) in N,N-dimethylformamide (DMF) (60 ml) was added 6-methoxy-2,5-dimethylpyridin-3-amine (1.882 g, 12.37 mmol), HATU (5.64 g, 14.84 mmol), and DIEA (6.48 ml, 37.1 mmol). The reaction mixture was stirred at room temperature for 1 hour, then diluted with water (110 mL) and extracted with EtOAc (3×150 mL). The combined organics were washed with water (3×100 mL), brine (100 mL), 10 dried over Na₂SO₄, filtered, and concentrated. The residue was triturated with DCM to afford 3.9 g of desired product as an off white solid. The mother liquor was concentrated down and purified by silica gel chromatography (CombiFlash, 120 g column) using 0-15% EtOAc/heptane as eluent to afford additional 2.1 g of desired product as an off white solid. The two batches were combined and dissolved in EtOAc and concentrated to afford N-(6-methoxy-2,5-dimethylpyridin-3-yl)-2-((2-methyl-4-(trifluoromethoxy)phenyl)amino)-5-(trifluoromethyl)nicotinamide (6.1 g, 11.86 mmol, 96% yield) as an off white solid. HPLC/MS 1.57 min (Method A), [M+H]⁺ 515.1. ¹H NMR (DMSO-d₆, 400 MHz) δ 10.87 (s, 1H), 10.42 (s, 1H), 8.67 (s, 2H), 8.19 (d, 1H, J=8.8 Hz), 7.48 (s, 1H), 7.3-7.3 (m, 1H), 7.23 (br d, 1H, J=8.8 Hz), 3.90 (s, 3H), 2.33 (s, 3H), 2.28 (s, 3H), 2.14 (s, 3H).

### Step C: 3-(6-methoxy-2,5-dimethylpyridin-3-yl)-1-(2-methyl-4-(trifluoromethoxy)phenyl)-6-(trifluoromethyl)-2,3-dihydropyrido[2,3-d]pyrimidin-4(1H)-one

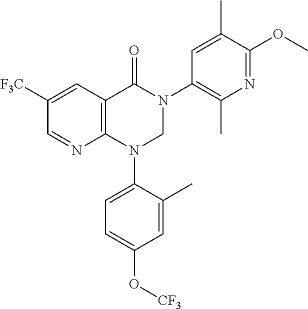 [SMILES: COc1nc(C)c(N2CN(c3ccc(OC(F)(F)F)cc3C)c3ncc(C(F)(F)F)cc3C2=O)cc1C]

To a solution of N-(6-methoxy-2,5-dimethylpyridin-3-yl)-2-((2-methyl-4-(trifluoromethoxy)phenyl)amino)-5-(trifluoromethyl)nicotinamide (8.1 g, 15.75 mmol) in acetonitrile (110 ml) was added cesium carbonate (20.52 g, 63.0 mmol) and diiodomethane (5.08 ml, 63.0 mmol). The reaction mixture was heated at 90° C. for 46 hours, cooled to room temperature, and the resulting solid was filtered and washed with EtOAc. The filtrate was concentrated and purified by silica gel chromatography (CombiFlash, 220 g column) using 0-20% EtOAc/heptane as eluent to afford 3-(6-methoxy-2,5-dimethylpyridin-3-yl)-1-(2-methyl-4-(trifluoromethoxy)phenyl)-6-(trifluoromethyl)-2,3-dihydropyrido[2,3-d]pyrimidin-4(1H)-one (5.0 g, 8.74 mmol, 55.5% yield) as a yellow solid. HPLC/MS 1.48 min (Method B), [M+H]⁺ 527.2. ¹H NMR (DMSO-d₆, 400 MHz) δ 8.62 (d, 1H, J=1.5 Hz), 8.34 (d, 1H, J=2.4 Hz), 7.4-7.5 (m, 3H), 7.31 (br d, 1H, J=8.3 Hz), 5.0-5.8 (m, 2H), 3.89 (s, 3H), 2.30 (br s, 3H), 2.25 (s, 3H), 2.12 (s, 3H).

### Step D: 3-(2,5-dimethyl-6-oxo-1,6-dihydropyridin-3-yl)-1-(2-methyl-4-(trifluoromethoxy)phenyl)-6-(trifluoromethyl)-2,3-dihydropyrido[2,3-d]pyrimidin-4(1H)-one

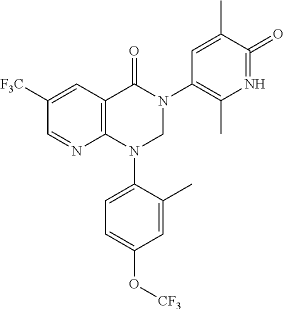

To a solution of 3-(6-methoxy-2,5-dimethylpyridin-3-yl)-1-(2-methyl-4-(trifluoromethoxy)phenyl)-6-(trifluoromethyl)-2,3-dihydropyrido[2,3-d]pyrimidin-4(1H)-one (4.9 g, 8.56 mmol) in isopropanol (100 mL) was added HCl (5-6 N in IPA) (42.8 mL, 214 mmol). The reaction mixture was heated at 90° C. for 3 hours, then cooled and concentrated. The residue was dissolved in EtOAc and neutralized with aq. sat. NaHCO₃. The organic layer was washed with brine, dried over Na₂SO₄, concentrated and purified by silica gel chromatography (CombiFlash, 120 g column) using 0-10% MeOH/DCM as eluent to afford 3-(2,5-dimethyl-6-oxo-1,6-dihydropyridin-3-yl)-1-(2-methyl-4-(trifluoromethoxy)phenyl)-6-(trifluoromethyl)-2,3-dihydropyrido[2,3-d]pyrimidin-4(1H)-one (4.18 g, 8.16 mmol, 95% yield) as an off white solid. HPLC/MS 1.14 min (Method B), [M+H]⁺ 513.1. ¹H NMR (DMSO-d₆, 400 MHz) δ 11.73 (s, 1H), 8.60 (d, 1H, J=2.0 Hz), 8.31 (d, 1H, J=2.0 Hz), 7.4-7.5 (m, 2H), 7.3-7.3 (m, 2H), 4.9-5.7 (m, 2H), 2.24 (s, 3H), 2.10 (br s, 3H), 1.94 (s, 3H).

### Step E: 3-(1-(chloromethyl)-2,5-dimethyl-6-oxo-1,6-dihydropyridin-3-yl)-1-(2-methyl-4-(trifluoromethoxy)phenyl)-6-(trifluoromethyl)-2,3-dihydropyrido[2,3-d]pyrimidin-4(1H)-one

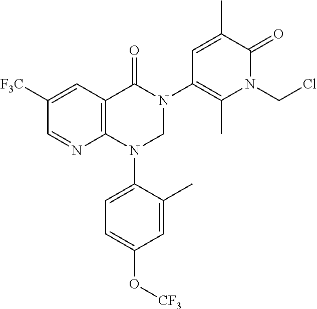 [SMILES: Cc1cc(OC(F)(F)F)ccc1N1CN(c2cc(C)c(=O)n(CCl)c2C)C(=O)c2cc(C(F)(F)F)cnc21]

To a solution of 3-(2,5-dimethyl-6-oxo-1,6-dihydropyridin-3-yl)-1-(2-methyl-4-(trifluoromethoxy)phenyl)-6-(trifluoromethyl)-2,3-dihydropyrido[2,3-d]pyrimidin-4(1H)-one (3.82 g, 7.45 mmol) and DABCO (1.254 g, 11.18 mmol) in ethyl acetate (60 ml) and N,N-Dimethylformamide (DMF) (7 ml), was added a solution of chloromethyl chloroformate (2.65 ml, 29.8 mmol) in ethyl acetate (10 ml) dropwise and a white precipitate formed. The reaction mixture was heated at 85° C. for 19 hours, and more chloromethyl chloroformate (1.326 ml, 14.91 mmol) was added and stirred at 85° C. for additional 3 hours, then more chloromethyl chloroformate (0.331 ml, 3.73 mmol) was added and stirred at 85° C. for another 1 hour. The reaction mixture was cooled, then diluted with more EtOAc and quenched with water. The layers were separated, the aqueous layer was extracted with EtOAc (3×). The combined organics were washed with sat. aq NaHCO₃, brine and dried over Na₂SO₄ and concentrated. The residue was purified by silica gel chromatography (Combiflash, 80 g column) eluting with 100% heptane for 5 minutes, then 10-60% EtOAc/heptane to afford 3-(1-(chloromethyl)-2,5-dimethyl-6-oxo-1,6-dihydropyridin-3-yl)-1-(2-methyl-4-(trifluoromethoxy)phenyl)-6-(trifluoromethyl)-2,3-dihydropyrido[2,3-d]pyrimidin-4(1H)-one (4.0 g, 7.13 mmol, 96% yield) as a white solid. HPLC/MS 1.31 min (Method B), [M+H]⁺ 561.1. ¹H NMR (DMSO-d₆, 400 MHz) δ 8.61 (s, 1H), 8.32 (d, 1H, J=2.4 Hz), 7.4-7.5 (m, 3H), 7.31 (br d, 1H, J=8.3 Hz), 4.9-6.4 (m, 4H), 2.41 (s, 3H), 2.24 (s, 3H), 2.0-2.0 (m, 3H).

### Step F: di-tert-butyl ((3,6-dimethyl-5-(1-(2-methyl-4-(trifluoromethoxy)phenyl)-4-oxo-6-(trifluoromethyl)-1,4-dihydropyrido[2,3-d]pyrimidin-3(2H)-yl)-2-oxopyridin-1(2H)-yl)methyl) phosphate

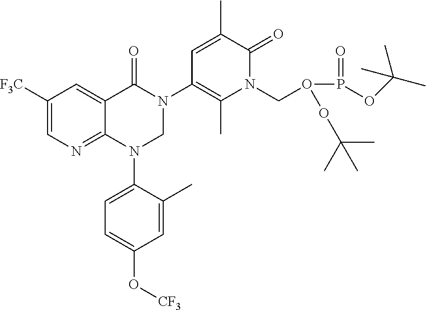

To a solution of 3-(1-(chloromethyl)-2,5-dimethyl-6-oxo-1,6-dihydropyridin-3-yl)-1-(2-methyl-4-(trifluoromethoxy)phenyl)-6-(trifluoromethyl)-2,3-dihydropyrido[2,3-d]pyrimidin-4(1H)-one (4.4 g, 7.84 mmol) and potassium di-tert-butyl phosphate (2.92 g, 11.77 mmol) in N,N-dimethylformamide (DMF) (110 ml) was added tetrabutylammonium iodide (0.290 g, 0.784 mmol) and heated at 70° C. for 3 hours. The reaction mixture was cooled and quenched with water (300 ml) when a white precipitate formed. The slurry was stirred vigorously for 1 hour. The solid was filtered and washed with water to afford di-tert-butyl ((3,6-dimethyl-5-(1-(2-methyl-4-(trifluoromethoxy)phenyl)-4-oxo-6-(trifluoromethyl)-1,4-dihydropyrido[2,3-d]pyrimidin-3(2H)-yl)-2-oxopyridin-1(2H)-yl)methyl) phosphate (5.086 g, 6.72 mmol, 86% yield) as an off white fine powder. HPLC/MS 1.41 min (Method B), [M+H]⁺ 735.1. ¹H NMR (DMSO-d₆, 400 MHz) δ 8.61 (br s, 1H), 8.32 (d, 1H, J=2.4 Hz), 7.4-7.5 (m, 3H), 7.31 (br d, 1H, J=7.8 Hz), 5.84 (br d, 2H, J=5.9 Hz), 4.8-5.7 (m, 2H), 2.36 (s, 3H), 2.24 (s, 3H), 2.00 (br s, 3H), 1.41 (s, 18H).

### Step G: (3,6-dimethyl-5-(1-(2-methyl-4-(trifluoromethoxy)phenyl)-4-oxo-6-(trifluoromethyl)-1,4-dihydropyrido[2,3-d]pyrimidin-3(2H)-yl)-2-oxopyridin-1(2H)-yl)methyl dihydrogen phosphate

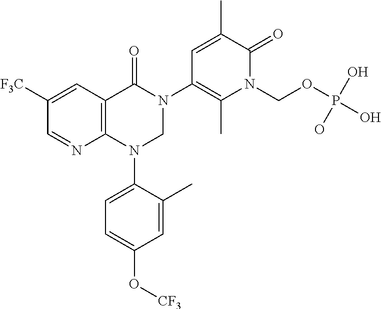

A slurry of di-tert-butyl ((3,6-dimethyl-5-(1-(2-methyl-4-(trifluoromethoxy)phenyl)-4-oxo-6-(trifluoromethyl)-1,4-dihydropyrido[2,3-d]pyrimidin-3(2H)-yl)-2-oxopyridin-1(2H)-yl)methyl) phosphate (5.0 g, 6.81 mmol) in acetone (50 mL) and water (40 mL) was heated at 50° C. for 26 hours. The clear reaction mixture was cooled and concentrated down under reduced pressure at 50° C. and dried in a vacuum oven at 45° C. overnight to afford 4.2 g of an off white glass solid. The solid was triturated with ether to afford 3.4 g of a white solid, which was triturated again with DCM:ether (1:2) to afford (3,6-dimethyl-5-(1-(2-methyl-4-(trifluoromethoxy)phenyl)-4-oxo-6-(trifluoromethyl)-1,4-dihydropyrido[2,3-d]pyrimidin-3(2H)-yl)-2-oxopyridin-1(2H)-yl)methyl dihydrogen phosphate (3.10 g, 4.83 mmol, 71% yield) as a white solid. HPLC/MS 0.78 min (Method B), [M+H]⁺ 623.0. ¹H NMR (DMSO-d₆, 400 MHz) δ 8.61 (br s, 1H), 8.32 (d, 1H, J=2.0 Hz), 7.4-7.5 (m, 3H), 7.32 (br d, 1H, J=8.3 Hz), 5.7-5.8 (m, 2H), 4.9-5.7 (m, 2H), 2.37 (s, 3H), 2.25 (s, 3H), 2.00 (br s, 3H) (two OH protons hidden); ¹H NMR (METHANOL-d₄, 400 MHz) δ 8.5-8.5 (m, 2H), 7.4-7.5 (m, 2H), 7.34 (s, 1H), 7.26 (br d, 1H, J=7.8 Hz), 6.01 (br d, 2H, J=6.8 Hz), 4.9-5.7 (m, 2H), 2.52 (s, 3H), 2.33 (s, 3H), 2.13 (s, 3H).

### Example 3: (5-(1-(3,4-Difluoro-2-methylphenyl)-4-oxo-6-(trifluoromethyl)-1,4-dihydropyrido[3,4-d]pyrimidin-3(2H)-yl)-6-methyl-2-oxopyridin-1(2H)-yl)methyl dihydrogen phosphate

### Step A: 3-(1-(Chloromethyl)-2-methyl-6-oxo-1,6-dihydropyridin-3-yl)-1-(3,4-difluoro-2-methylphenyl)-6-(trifluoromethyl)-2,3-dihydropyrido[3,4-d]pyrimidin-4(1H)-one

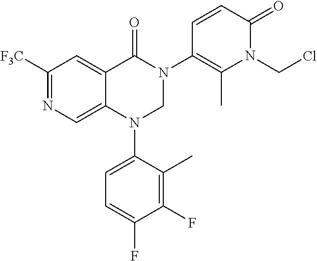 [SMILES: Cc1c(N2CN(c3ccc(=O)n(CCl)c3C)C(=O)c3cc(C(F)(F)F)ncc32)ccc(F)c1F]

To a solution of 1-(3,4-difluoro-2-methylphenyl)-3-(2-methyl-6-oxo-1,6-dihydropyridin-3-yl)-6-(trifluoromethyl)-2,3-dihydropyrido[3,4-d]pyrimidin-4(1H)-one (0.162 g, 0.360 mmol) and DABCO (0.032 g, 0.288 mmol) in ethyl acetate (3.27 ml) and DMF (0.327 ml) under nitrogen, was added chloromethyl chloroformate (0.080 ml, 0.899 mmol) dropwise and was stirred at room temperature for 2 hr and then at 60° C. for 18 hr. Additional chloromethyl chloroformate (0.056 ml, 0.719 mmol) and DABCO (0.032 g, 0.288 mmol) were added and the reaction was heated for 2 hr. Additional chloromethyl chloroformate (0.278 ml, 3.60 mmol) was added and continued heating for 18 hr. The reaction was cooled and quenched with sat. aq. NaHCO₃. The aqueous layer was extracted with EtOAc (2×). The combined organics were washed with water, brine and dried with MgSO₄ and the solvent concentrated. The residue was purified by flash column chromatography (Isco, 24 g column, 0-50% (3:1 EtOAc:EtOH)/heptane) to provide the title compound (110 mg, 0.221 mmol, 61% yield). ¹H NMR (DMSO-d₆, 501 MHz): δ (ppm) 8.02-8.06 (m, 1H, H-12), 7.84-8.00 (m, 1H, H-9), 7.41-7.58 (m, 2H, H-5, 17), 7.36 (br d, J=18.4 Hz, 1H, H-6), 6.44 (d, J=9.8 Hz, 1H, H-18), 5.57-6.32 (m, 2H, H-20), 4.85-5.56 (m, 2H, H-15), 2.31-2.48 (m, 3H, H-21), 2.23 (s, 3H, H-1). MS (m/z) 499 (M+H⁺).

### Step B: Di-tert-butyl ((5-(1-(3,4-difluoro-2-methylphenyl)-4-oxo-6-(trifluoromethyl)-1,4-dihydropyrido[3,4-d]pyrimidin-3(2H)-yl)-6-methyl-2-oxopyridin-1(2H)-yl)methyl) phosphate

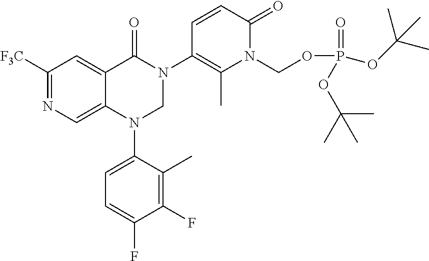 [SMILES: Cc1c(N2CN(c3ccc(=O)n(COP(=O)(OC(C)(C)C)OC(C)(C)C)c3C)C(=O)c3cc(C(F)(F)F)ncc32)ccc(F)c1F]

To a solution of 3-(1-(chloromethyl)-2-methyl-6-oxo-1,6-dihydropyridin-3-yl)-1-(3,4-difluoro-2-methylphenyl)-6-(trifluoromethyl)-2,3-dihydropyrido[3,4-d]pyrimidin-4(1H)-one (0.091 g, 0.182 mmol) and potassium di-tert-butyl phosphate (0.068 g, 0.274 mmol) in DMF (0.91 ml) was added tetrabutylammonium iodide (6.74 mg, 0.018 mmol) and heated at 70° C. for 2 hr. The reaction thickened so additional DMF (0.5 ml) was added. The reaction was then cooled and quenched with water and diluted with EtOAc and water. The layers were separated and the aqueous layer was extracted with EtOAc (3×). The combined organics were washed with water, brine and dried with MgSO₄ and concentrated. The residue was purified by flash column chromatography (Isco, 24 g column, 0-50% (3:1 EtOAc:EtOH)/heptane) to provide the title compound (95 mg, 0.141 mmol, 77% yield). ¹H NMR (DMSO-d₆, 400 MHz) δ 8.05 (s, 1H), 7.9-8.0 (m, 1H), 7.3-7.5 (m, 3H), 6.43 (d, 1H, J=9.3 Hz), 5.7-5.9 (m, 2H), 4.9-5.6 (m, 2H), 2.3-2.4 (m, 3H), 2.24 (d, 3H, J=2.0 Hz), 1.42 (s, 18H). MS (m/z) 561 (M+H−(t-butyl)).

### Step C: (5-(1-(3,4-Difluoro-2-methylphenyl)-4-oxo-6-(trifluoromethyl)-1,4-dihydropyrido[3,4-d]pyrimidin-3(2H)-yl)-6-methyl-2-oxopyridin-1(2H)-yl)methyl dihydrogen phosphate

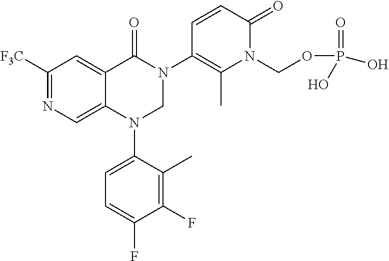 [SMILES: Cc1c(N2CN(c3ccc(=O)n(COP(=O)(O)O)c3C)C(=O)c3cc(C(F)(F)F)ncc32)ccc(F)c1F]

To a solution of di-tert-butyl ((5-(1-(3,4-difluoro-2-methylphenyl)-4-oxo-6-(trifluoromethyl)-1,4-dihydropyrido[3,4-d]pyrimidin-3(2H)-yl)-6-methyl-2-oxopyridin-1(2H)-yl)methyl) phosphate (88.8 mg, 0.132 mmol) dissolved in acetonitrile (660 μl) and water (660 μl) was added acetic acid (151 μl, 2.64 mmol) and stirred at 70° C. for 1.5 hr. The solvents were then concentrated and reaction purified by reverse phase chromatography on a XSELECT CSH C18 column (150 mm×30 mm i.d. 5 μm packing diameter) at ambient temperature with 0.1% formic acid in acetonitrile in 0.10% formic acid in water as eluant, to afford the title compound (46 mg, 0.081 mmol, 61% yield). ¹H NMR (DMSO-d₆, 400 MHz) δ 8.05 (s, 1H), 7.9-8.0 (m, 1H), 7.3-7.5 (m, 3H), 6.41 (d, 1H, J=9.8 Hz), 5.75 (br d, 2H, J=4.4 Hz), 4.9-5.6 (m, 2H), 2.3-2.4 (m, 3H), 2.24 (d, 3H, J=2.0 Hz), phosphate hydroxy protons exchanged. MS (m/z) 559 (M−H)⁻.

### Example 4: (5-(7-chloro-1-(3,4-difluoro-2-methylphenyl)-6-fluoro-4-oxo-1,4-dihydroquinazolin-3(2H)-yl)-6-methyl-2-oxopyridin-1(2H)-yl)methyl dihydrogen phosphate

### Step A: 4-chloro-2-((3,4-difluoro-2-methylphenyl)amino)-5-fluorobenzoic acid

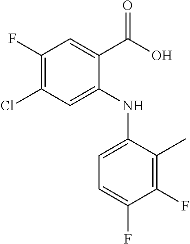 [SMILES: Cc1c(Nc2cc(Cl)c(F)cc2C(=O)O)ccc(F)c1F]

To 1-bromo-3,4-difluoro-2-methylbenzene (8.52 g, 41.1 mmol) in anhydrous degassed 1,4-dioxane (176 ml) under nitrogen was added 2-amino-4-chloro-5-fluorobenzoic acid (6 g, 31.7 mmol), cesium carbonate (25.8 g, 79 mmol), BINAP (1.971 g, 3.17 mmol), and Pd₂(dba)₃ (1.449 g, 1.583 mmol). The reaction was stirred at 95° C. for 3 days. The brown suspension was cooled and diluted with ethyl acetate (350 mL) and filtered through Celite. The filter cake was washed with EtOAc (3×200 mL). The filtrate was concentrated in vacuo at 40° C. to a dark oil and a mixture of 1:1 DCM:heptane (150 mL) was added to give a precipitate. The solid was collected by vacuum filtration, washed with 10% DCM/heptane (3×25 mL) and dried in air under vacuum to give 4-chloro-2-((3,4-difluoro-2-methylphenyl)amino)-5-fluorobenzoic acid, Cesium salt (2.78 g, 5.58 mmol, 17.6% yield) as a tan-orange solid. The resulting Celite filter cake containing additional product was transferred to a 1 L beaker and stirred with 200 mL water at 25° C. and the pH adjusted to 4-5 with 1 N HCl when DCM (500 mL) was added. The celite mixture was filtered, the solid kept for further isolation of product. The layers of the filtrate were separated, the organic layer was dried over anhydrous sodium sulfate, filtered, and concentrated at 40° C. to an orange-yellow solid as desired product, 4-chloro-2-((3,4-difluoro-2-methylphenyl)amino)-5-fluorobenzoic acid (0.5 g, 1.426 mmol, 4.5% yield) as an orange-yellow solid. The green Celite filter cake solid from above was transferred to a 1 L beaker and MeOH (400 mL), MeCN (100 mL) and DCM (50 mL) were added and the mixture stirred 1 hour at 25° C. The solid was collected by vacuum filtration and rinsed with MeOH (4×25 mL). The filtrate was concentrated at 40° C. to a brown solid as additional desired product, 4-chloro-2-((3,4-difluoro-2-methylphenyl)amino)-5-fluorobenzoic acid (7.65 g, 23.02 mmol, 72.7% yield). HPLC/MS 1.37 min (Method A), [M+1]⁺ 315.9. ¹H NMR (400 MHz, DMSO-d₆) δ 12.16 (s, 1H), 7.74 (d, J=10.3 Hz, 1H), 7.19 (q, J=9.3 Hz, 1H), 7.13-7.07 (m, 1H), 6.91 (d, J=6.4 Hz, 1H), 2.16 (d, J=2.4 Hz, 3H).

### Step B: 4-chloro-2-((3,4-difluoro-2-methylphenyl)amino)-5-fluoro-N-(6-methoxy-2-methylpyridin-3-yl)benzamide

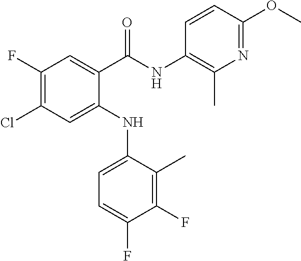 [SMILES: COc1ccc(NC(=O)c2cc(F)c(Cl)cc2Nc2ccc(F)c(F)c2C)c(C)n1]

To a brown suspension of 4-chloro-2-((3,4-difluoro-2-methylphenyl)amino)-5-fluorobenzoic acid (8.15 g, 25.8 mmol) in N,N-dimethylformamide (148 ml) was added 6-methoxy-2-methylpyridin-3-amine (4.46 g, 32.3 mmol) and HATU (11.78 g, 31.0 mmol), followed by DIEA (13.53 ml, 77 mmol) portion wise over 30 minutes to give a dark brown solution. This was stirred for 5 min at 25° C. and combined with another batch (2.70 g, 6.18 mmol crude product) for work up and purification. To the crude reaction mixtures, water (320 mL) was added portion wise with stirring, and the mixture was extracted with EtOAc (400 mL, then 100 mL). The combined organic layers were washed with water (2×200 mL), then brine (50 mL). The organic layer was dried over anhydrous sodium sulfate and concentrated in vacuo at 40° C. to a brown oil. To this oil was added 10 mL DCM to form a granular solid, and 1:1 DCM:heptane (100 mL) was added stirred for 3 hr to break up the solid. The solid was collected by vacuum filtration, the cake rinsed with 10% DCM/heptane (4×10 mL), and dried overnight under vacuum providing the title compound 4-chloro-2-((3,4-difluoro-2-methylphenyl)amino)-5-fluoro-N-(6-methoxy-2-methylpyridin-3-yl)benzamide (3.18 g, 6.93 mmol, 21.67% yield) as a tan solid. The filtrate was treated with 10 mL DCM, then 1:1 DCM:heptane (100 mL) was added and stirred for 1 hr to break up the solid. The solid was collected by vacuum filtration, rinsed with heptane (4×10 mL) and dried under vacuum in air to provide additional product 4-chloro-2-((3,4-difluoro-2-methylphenyl)amino)-5-fluoro-N-(6-methoxy-2-methylpyridin-3-yl)benzamide (7.005 g, 15.27 mmol, 47.7% yield) as a brown solid. The resulting filtrate was purified on a 220 gram ISCO Gold silica column (Isco CombiFlash Rf, eluting with 0 to 25% EtOAc/heptane, 10 min gradient, 125 mL/min). Pure fractions were concentrated at 40° C. to an orange solid and dried under high vacuum to provide title compound 4-chloro-2-((3,4-difluoro-2-methylphenyl)amino)-5-fluoro-N-(6-methoxy-2-methylpyridin-3-yl)benzamide (1.744 g, 3.80 mmol, 11.88% yield) as a bright orange solid. HPLC/MS 1.32 min (Method A), [M+H]⁺ 436.0. ¹H NMR (400 MHz, DMSO-d6) δ 10.11 (s, 1H), 9.27 (s, 1H), 7.96 (d, J=10.3 Hz, 1H), 7.60 (d, J=8.8 Hz, 1H), 7.28 (q, J=9.3 Hz, 1H), 7.18-7.08 (m, 1H), 6.92 (d, J=6.8 Hz, 1H), 6.69 (d, J=8.3 Hz, 1H), 3.85 (s, 3H), 2.35 (s, 3H), 2.12 (d, J=2.0 Hz, 3H).

### Step C: 7-chloro-1-(3,4-difluoro-2-methylphenyl)-6-fluoro-3-(6-methoxy-2-methylpyridin-3-yl)-2,3-dihydroquinazolin-4(1H)-one

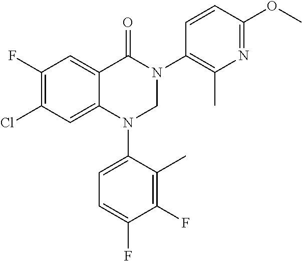 [SMILES: COc1ccc(N2CN(c3ccc(F)c(F)c3C)c3cc(Cl)c(F)cc3C2=O)c(C)n1]

To 4-Chloro-2-((3,4-difluoro-2-methylphenyl)amino)-5-fluoro-N-(6-methoxy-2-methylpyridin-3-yl)benzamide (11.42 g, 26.2 mmol) in chloroform (262 ml) were added formaldehyde (1.967 g, 65.5 mmol) and sulfuric acid (3.49 ml, 65.5 mmol). The reaction mixture was stirred at 55° C. for 1 hr, affording an orange gum on completion. Saturated sodium bicarbonate (25 mL) was added, the organic layer separated and the aqueous extracted 2×25 mL EtOAc. The combined organics were combined, dried over anhydrous MgSO₄, and concentrated in vacuo to a brown foam. This was combined with crude product from another batch (1.78 g, 3.99 mmol) for purification. This combined solid was dissolved in 20 mL DCM and was purified via Isco CombiFlash Rf (0% to 50% in EtOAc in Heptane; 330 g column, 25 min gradient). The pure fractions were collected and the product isolated by concentration in vacuo to provide 7-chloro-1-(3,4-difluoro-2-methylphenyl)-6-fluoro-3-(6-methoxy-2-methylpyridin-3-yl)-2,3-dihydroquinazolin-4(1H)-one (11.5 g, 25.7 mmol, 85% yield based both batches). HPLC/MS 1.31 min (Method B), [M+1]⁺ 448.1. ¹H NMR (DMSO-d₆, 400 MHz) δ 7.79 (d, 1H, J=9.3 Hz), 7.59 (br d, 1H, J=7.8 Hz), 7.3-7.5 (m, 1H), 7.16 (br d, 1H, J=2.4 Hz), 6.71 (d, 1H, J=9.3 Hz), 6.4-6.6 (m, 1H), 4.7-5.6 (m, 2H), 3.83 (s, 3H), 2.2-2.4 (m, 3H), 2.20 (d, 3H, J=1.5 Hz).

### Step D: 7-chloro-1-(3,4-difluoro-2-methylphenyl)-6-fluoro-3-(2-methyl-6-oxo-1,6-dihydropyridin-yl)-2,3-dihydroquinazolin-4(1H)-one

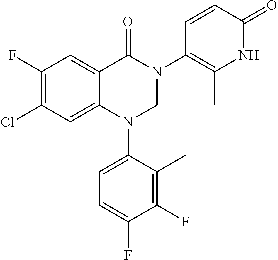 [SMILES: Cc1[nH]c(=O)ccc1N1CN(c2ccc(F)c(F)c2C)c2cc(Cl)c(F)cc2C1=O]

A solution of 7-chloro-1-(3,4-difluoro-2-methylphenyl)-6-fluoro-3-(6-methoxy-2-methylpyridin-3-yl)-2,3-dihydroquinazolin-4(1H)-one (11.43 g, 25.5 mmol) and HCl (128 ml, 638 mmol, 5N in isopropanol) in isopropanol (255 ml) was heated to 90° C. under a condenser for 1 hr when 1,2-dichloroethane (85 ml) was added and the solution was heated at 90° C. for 20 hr. The reaction was cooled and the solvent was removed in vacuo to give a white solid (9.9 g). The solid was dissolved in EtOAc and washed with sat. aq. sodium bicarbonate, dried organic over anhydrous magnesium sulfate and concentrated in vacuo to provide 7-chloro-1-(3,4-difluoro-2-methylphenyl)-6-fluoro-3-(2-methyl-6-oxo-1,6-dihydropyridin-yl)-2,3-dihydroquinazolin-4(1H)-one as a white solid (8.55 g, 19.7 mmol, 77% yield). HPLC/MS 1.08 min (Method B), [M+1]⁺ 434.1. ¹H NMR (DMSO-d₆, 400 MHz) δ 11.75 (br s, 1H), 7.77 (d, 1H, J=9.3 Hz), 7.35 (br dd, 2H, J=10.3, 19.1 Hz), 7.13 (br s, 1H), 6.4-6.8 (m, 1H), 6.17 (br d, 1H, J=9.3 Hz), 4.7-5.5 (m, 2H), 2.0-2.1 (s, 6H).

### Step E: 7-chloro-3-(1-(chloromethyl)-2-methyl-6-oxo-1,6-dihydropyridin-3-yl)-1-(3,4-difluoro-2-methylphenyl)-6-fluoro-2,3-dihydroquinazolin-4(1H)-one

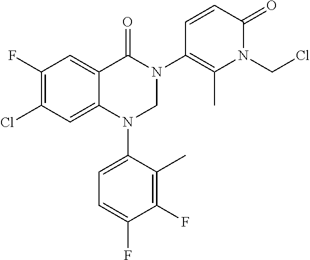 [SMILES: Cc1c(N2CN(c3ccc(=O)n(CCl)c3C)C(=O)c3cc(F)c(Cl)cc32)ccc(F)c1F]

To a solution of 7-chloro-1-(3,4-difluoro-2-methylphenyl)-6-fluoro-3-(2-methyl-6-oxo-1,6-dihydropyridin-3-yl)-2,3-dihydroquinazolin-4(1H)-one (4.6 g, 10.60 mmol) and DABCO (1.784 g, 15.91 mmol) in ethyl acetate (96 ml) and N,N-dimethylformamide (DMF) (9.64 ml) was added chloromethyl chloroformate (3.77 ml, 42.4 mmol) and heated to 90° C. for 36 hr followed by 25° C. for 3 days total. Additional chloromethyl chloroformate (3.77 ml, 42.4 mmol) was added and heated at 90° C. for 2 hr. The reaction was cooled and 100 mL water and 50 mL brine were added. The product was extracted into 100 mL EtOAc, the organic layer separated, the aqueous layer extracted with 75 mL EtOAc and the combined organic layers were washed with brine (2×), sat. bicarb solution, dried over anhydrous magnesium sulfate and concentrated in vacuo to a yellow solid (4.8 g). The solid was triturated with 10 mL DCM affording a white solid (3 g). The filtrate was filtered to yield a second crop. The solid was combined to provide (3.69 g 7.65 mmol, 72% yield) of an off white solid. The remaining filtrate was concentrated in vacuo, the residue dissolved in 1:1 hot EtOAc:MeOH and preabsorbed on silica. The residue was purified via Isco CombiFlash Rf (50% to 100% in EtOAc in heptane; 40 g column, 20 min gradient). The pure fractions were pooled and concentrated in vacuo to give additional title compound as a white solid (500 mg 1.04 mmol, 9.8% yield). HPLC/MS 1.25 min (Method B), [M+1]⁺ 482.1. ¹H NMR (DMSO-d₆, 400 MHz) δ 7.78 (d, 1H, J=9.3 Hz), 7.2-7.5 (m, 2H), 7.16 (br dd, 1H, J=3.9, 8.3 Hz), 6.4-6.8 (m, 1H), 6.41 (d, 1H, J=9.8 Hz), 5.5-6.3 (m, 2H), 4.7-5.4 (m, 2H), 2.2-2.5 (m, 3H), 2.20 (d, 3H, J=2.0 Hz).

### Step F: Di-tert-butyl ((5-(7-chloro-1-(3,4-difluoro-2-methylphenyl)-6-fluoro-4-oxo-1,4-dihydroquinazolin-3(2H)-yl)-6-methyl-2-oxopyridin-1(2H)-yl)methyl) phosphate

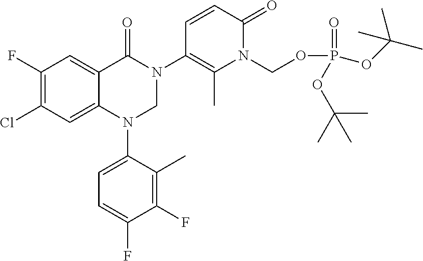 [SMILES: Cc1c(N2CN(c3ccc(=O)n(COP(=O)(OC(C)(C)C)OC(C)(C)C)c3C)C(=O)c3cc(F)c(Cl)cc32)ccc(F)c1F]

To a solution of 7-chloro-3-(1-(chloromethyl)-2-methyl-6-oxo-1,6-dihydropyridin-3-yl)-1-(3,4-difluoro-2-methylphenyl)-6-fluoro-2,3-dihydroquinazolin-4(1H)-one (4.1 g, 8.50 mmol) and potassium di-tert-butyl phosphate (3.69 g, 14.88 mmol) in N,N-dimethylformamide (121 ml) was added tetrabutylammonium iodide (0.314 g, 0.850 mmol) and heated at 70° C. for 1 hr. The slightly gummy mixture was quenched with 40 mL water and extracted into 40 mL EtOAc. The aqueous layer was extracted EtOAc (2×) and the combined organic layers were washed with water, sat. sodium bicarbonate solution, brine, dried over anhydrous magnesium sulfate and concentrated in vacuo to a yellow semi-solid. The solid was purified via Isco CombiFlash Rf (0-20% (3:1 EtOAc/EtOH) in DCM, 80 g silica column, 20 min gradient). The pure fractions were pooled and concentrated in vacuo to give di-tert-butyl ((5-(7-chloro-1-(3,4-difluoro-2-methylphenyl)-6-fluoro-4-oxo-1,4-dihydroquinazolin-3(2H)-yl)-6-methyl-2-oxopyridin-1(2H)-yl)methyl) phosphate as a solid. HPLC/MS 1.37 min (Method B), [M−2^{t}Bu]⁺ 544.0. ¹H NMR (DMSO-d₆, 400 MHz) δ 7.78 (d, 1H, J=9.3 Hz), 7.3-7.5 (m, 2H), 7.16 (br d, 1H, J=1.0 Hz), 6.4-6.8 (m, 1H), 6.38 (d, 1H, J=9.8 Hz), 5.7-5.9 (m, 2H), 4.7-5.5 (m, 2H), 2.2-2.4 (m, 3H), 2.1-2.2 (m, 3H), 1.3-1.5 (m, 18H).

### Step G: (5-(7-chloro-1-(3,4-difluoro-2-methylphenyl)-6-fluoro-4-oxo-1,4-dihydroquinazolin-3(2H)-yl)-6-methyl-2-oxopyridin-1(2H)-yl)methyl dihydrogen phosphate

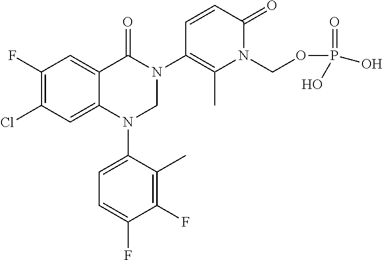 [SMILES: Cc1c(N2CN(c3ccc(=O)n(COP(=O)(O)O)c3C)C(=O)c3cc(F)c(Cl)cc32)ccc(F)c1F]

Di-tert-butyl ((5-(7-chloro-1-(3,4-difluoro-2-methylphenyl)-6-fluoro-4-oxo-1,4-dihydroquinazolin-3(2H)-yl)-6-methyl-2-oxopyridin-1(2H)-yl)methyl) phosphate (3.36 g, 5.12 mmol) was heated in acetone (26 ml) and water (26 ml) for 18 hr at 50° C. The reaction was cooled to give a colorless solution and evaporated in vacuo at 40° C. to a white foamy solid and dried on high vacuum to give (5-(7-chloro-1-(3,4-difluoro-2-methylphenyl)-6-fluoro-4-oxo-1,4-dihydroquinazolin-3(2H)-yl)-6-methyl-2-oxopyridin-1(2H)-yl)methyl dihydrogen phosphate (2.6 g, 4.78 mmol, 93% yield). HPLC/MS 0.73 min (Method B), [M+H]⁺ 544.0. ¹H NMR (DMSO-d₆, 400 MHz) δ 11.0-12.2 (m, 2H), 7.78 (d, 1H, J=9.3 Hz), 7.38 (br d, 2H, J=9.8 Hz), 7.1-7.2 (m, 1H), 6.5-6.8 (m, 1H), 6.37 (d, 1H, J=9.8 Hz), 5.6-5.8 (m, 2H), 4.6-5.5 (m, 2H), 2.2-2.4 (m, 3H), 2.20 (d, 3H, J=2.4 Hz).

### Example 5: (5-(7-Chloro-6-fluoro-1-(4-fluoro-2-methylphenyl)-4-oxo-1,4-dihydroquinazolin-3(2H)-yl)-6-methyl-2-oxopyridin-1(2H)-yl)methyl dihydrogen phosphate

### Step A: 7-Chloro-3-(1-(chloromethyl)-2-methyl-6-oxo-1,6-dihydropyridin-3-yl)-6-fluoro-1-(4-fluoro-2-methylphenyl)-2,3-dihydroquinazolin-4(1H)-one

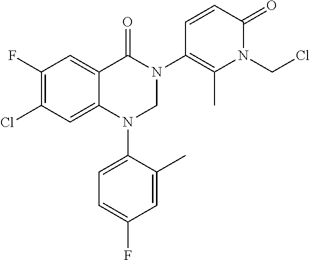 [SMILES: Cc1cc(F)ccc1N1CN(c2ccc(=O)n(CCl)c2C)C(=O)c2cc(F)c(Cl)cc21]

To a solution of 7-chloro-6-fluoro-1-(4-fluoro-2-methylphenyl)-3-(2-methyl-6-oxo-1,6-dihydropyridin-3-yl)-2,3-dihydroquinazolin-4(1H)-one (5.06 g, 12.17 mmol) and DABCO (0.683 g, 6.08 mmol) in Ethyl acetate (111 ml) and N,N-Dimethylformamide (DMF) (11.06 ml) under nitrogen, was added chloromethyl chloroformate (2.164 ml, 24.34 mmol) dropwise via addition funnel and heated to 75° C. for 19 h. The reaction was cooled, quenched with water and the layers were separated. The aqueous layer was back extracted with EtOAc (2×). The combined organics were washed with sat. aq NaHCO₃ and dried with MgSO₄ and concentrated. The residue was purified by flash column chromatography (Isco, 300 g column, 0-50% (3:1 EtOAc:EtOH)/Heptane) to provide the title compound (3.88 g, 8.19 mmol, 67% yield). ¹H NMR (DMSO-d₆, 400 MHz) δ 7.78 (d, 1H, J=9.3 Hz), 7.47 (d, 1H, J=9.8 Hz), 7.3-7.4 (m, 2H), 7.1-7.2 (m, 1H), 6.42 (d, 1H, J=9.8 Hz), 6.3-6.4 (m, 1H), 5.5-6.2 (m, 2H), 4.7-5.5 (m, 2H), 2.3-2.5 (m, 3H), 2.26 (s, 3H), MS (m/z) 464 (M+H)⁺.

### Step B: Di-tert-butyl ((5-(7-chloro-6-fluoro-1-(4-fluoro-2-methylphenyl)-4-oxo-1,4-dihydroquinazolin-3(2H)-yl)-6-methyl-2-oxopyridin-1(2H)-yl)methyl) phosphate

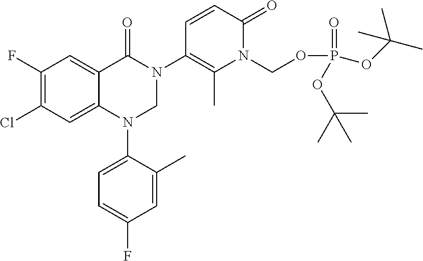 [SMILES: Cc1cc(F)ccc1N1CN(c2ccc(=O)n(COP(=O)(OC(C)(C)C)OC(C)(C)C)c2C)C(=O)c2cc(F)c(Cl)cc21]

To a solution of 7-chloro-3-(1-(chloromethyl)-2-methyl-6-oxo-1,6-dihydropyridin-3-yl)-6-fluoro-1-(4-fluoro-2-methylphenyl)-2,3-dihydroquinazolin-4(1H)-one (3.52 g, 7.58 mmol) and potassium di-tert-butyl phosphate (2.82 g, 11.37 mmol) in N,N-Dimethylformamide (DMF) (100 ml) was added tetrabutylammonium iodide (0.280 g, 0.758 mmol) and heated at 70° C. for 2.5 h. The reaction was cooled, quenched with water and diluted with EtOAc and water. The layers were separated and the aqueous layer was extracted with EtOAc (3×). The combined organics were washed with water, brine and dried with MgSO₄, and concentrated. The residue was purified by flash column chromatography (Isco, 300 g column, 0-20% (3:1 EtOAc:EtOH)/DCM) to provide the title compound (3.52 g, 5.35 mmol, 70% yield). ¹H NMR (DMSO-d₆, 400 MHz) δ 7.78 (d, 1H, J=8.8 Hz), 7.45 (d, 1H, J=9.8 Hz), 7.3-7.4 (m, 2H), 7.16 (br s, 1H), 6.3-6.6 (m, 2H, J=9.8 Hz), 5.7-5.9 (m, 2H), 4.6-5.5 (m, 2H), 2.3-2.4 (m, 3H), 2.26 (s, 3H), 1.41 (d, 18H, J=2.4 Hz), MS (m/z) 638 (M+H)⁺.

### Step C: (5-(7-Chloro-6-fluoro-1-(4-fluoro-2-methylphenyl)-4-oxo-1,4-dihydroquinazolin-3(2H)-yl)-6-methyl-2-oxopyridin-1(2H)-yl)methyl dihydrogen phosphate

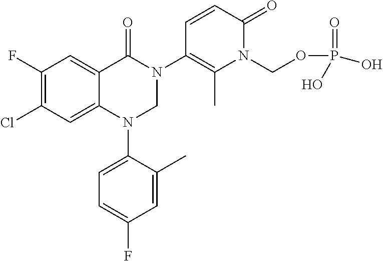 [SMILES: Cc1cc(F)ccc1N1CN(c2ccc(=O)n(COP(=O)(O)O)c2C)C(=O)c2cc(F)c(Cl)cc21]

A solution of di-tert-butyl ((5-(7-chloro-6-fluoro-1-(4-fluoro-2-methylphenyl)-4-oxo-1,4-dihydroquinazolin-3(2H)-yl)-6-methyl-2-oxopyridin-1(2H)-yl)methyl) phosphate (3.47 g, 5.44 mmol) dissolved in Acetone (40 ml) and Water (30 ml) was heated to 50° C. for 16 h, and stirred at room temperature for 6 h. The solvents were concentrated and then was pumped under high vacuum to provide the title compound (2.74 g, 5.16 mmol, 95% yield). ¹H NMR (DMSO-d₆, 400 MHz) δ 7.78 (d, 1H, J=9.3 Hz), 7.44 (d, 1H, J=9.8 Hz), 7.3-7.4 (m, 2H), 7.18 (br d, TH, J=6.8 Hz), 6.3-6.5 (in, 2H, J=9.8 Hz), 5.7-5.8 (m, 2H), 4.6-5.6 (m, 2H), 2.8-4.3 (m, 2H), 2.3-2.4 (m, 3H), 2.26 (s, 3H), MS (m/z) 526 (M+H)⁺.

TABLE 1

| Compound | Name | Structure | Characterization |
|---|---|---|---|
| 1 | (5-(7-fluoro-1-(4-fluoro-2- methylphenyl)-4-oxo-6- (trifluoromethyl)-1,4- dihydroquinazolin-3(2H)- yl)-6-methyl-2- oxopyridin-1(2H)- yl)methyl dihydrogen phosphate | 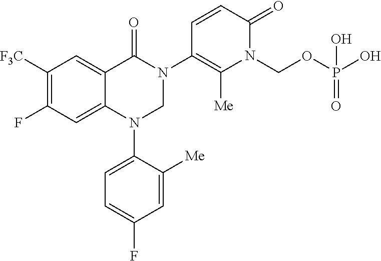 [SMILES: Cc1cc(F)ccc1N1CN(c2ccc(=O)n(COP(=O)(O)O)c2C)C(=O)c2cc(C(F)(F)F)c(F)cc21] | [M + H]⁺ 560.2. ¹H NMR (METHANOL- d₄, 400 MHz) δ 8.25 (d, 1H, J = 7.8 Hz), 7.5-7.6 (m, 1H), 7.4- 7.4 (m, 1H), 7.2-7.3 (m, 1H), 7.1-7.2 (m, 1H), 6.53 (d, 1H, J = 9.8 Hz), 6.1-6.2 (m, 1H), 5.99 (d, 2H, J = 6.8 Hz), 4.8-5.6 (m, 2H), 2.5-2.6 (m, 3H), 2.3-2.4 (m, 3H). 1H NMR (DMSO-d₆, 400 MHz) δ 8.10 (d, 1H, J = 8.3 Hz), 7.4-7.5 |
| | | | (m, 2H), 7.3-7.4 (m, |
| | | | 1H), 7.2-7.3 (m, 1H), |
| | | | 6.4-6.4 (m, 1H), 6.14 |
| | | | (dd, 1H, J = 13.0, 18.3 Hz), |
| | | | 5.7-5.8 (m, 2H), |
| | | | 4.7-5.6 (m, 2H), 2.3- |
| | | | 2.4 (m, 3H), 2.2-2.3 |
| | | | (m, 3H) (two OH |
| | | | protons hidden). |
| 2 | (3,6-dimethyl-5-(1-(2- methyl-4- (trifluoromethoxy)phenyl)- 4-oxo-6-(trifluoromethyl)- 1,4-dihydropyrido[2,3- d]pyrimidin-3(2H)-yl)-2- oxopyridin-1(2H)- yl)methyl dihydrogen phosphate | 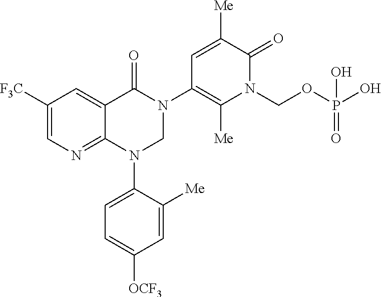 [SMILES: Cc1cc(OC(F)(F)F)ccc1N1CN(c2cc(C)c(=O)n(COP(=O)(O)O)c2C)C(=O)c2cc(C(F)(F)F)cnc21] | [M + H]⁺ 623.0. ¹H NMR (DMSO-d₆, 400 MHz) δ 8.61 (br s, 1H), 8.32 (d, 1H, J = 2.0 Hz), 7.4-7.5 (m, 3H), 7.32 (br d, 1H, J = 8.3 Hz), 5.7-5.8 (m, 2H), 4.9-5.7 (m, 2H), 2.37 (s, 3H), 2.25 (s, 3H), 2.00 (br s, 3H) (two OH protons hidden). ¹H NMR (METHANOL-d₄, 400 MHz) δ 8.5-8.5 (m, 2H), 7.4-7.5 (m, 2H), 7.34 (s, 1H), 7.26 (br d, 1H, J = 7.8 Hz), 6.01 (br d, 2H, J = 6.8 Hz), |
| | | | 4.9-5.7 (m, 2H), 2.52 |
| | | | (s, 3H), 2.33 (s, 3H), |
| | | | 2.13 (s, 3H). |
| 3 | (5-(1-(3,4-difluoro-2- methylphenyl)-4-oxo-6- (trifluoromethyl)-1,4- dihydropyrido[3,4- d]pyrimidin-3(2H)-yl)-6- methyl-2-oxopyridin- 1(2H)-yl)methyl dihydrogen phosphate | 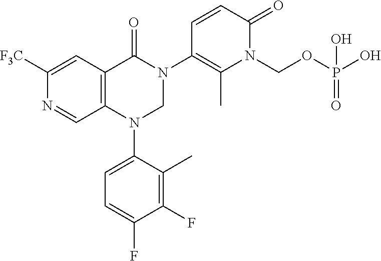 [SMILES: Cc1c(N2CN(c3ccc(=O)n(COP(=O)(O)O)c3C)C(=O)c3cc(C(F)(F)F)ncc32)ccc(F)c1F] | ¹H NMR (DMSO-d₆, 400 MHz) δ 8.05 (s, 1H), 7.9-8.0 (m, 1H), 7.3-7.5 (m, 3H), 6.41 (d, 1H, J = 9.8 Hz), 5.75 (br d, 2H, J = 4.4 Hz), 4.9-5.6 (m, 2H), 2.3-2.4 (m, 3H), 2.24 (d, 3H, J = 2.0 Hz), phosphate hydroxy protons exchanged. MS (m/z) 559 (M − H)⁻ |
| 4 | (5-(7-chloro-1-(3,4- difluoro-2-methylphenyl)- 6-fluoro-4-oxo-1,4- dihydroquinazolin-3(2H)- yl)-6-methyl-2- oxopyridin-1(2H)- yl)methyl dihydrogen phosphate | 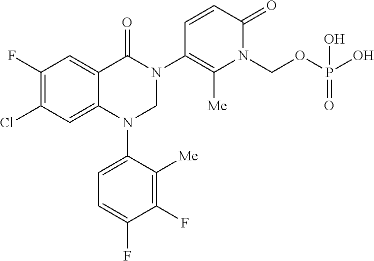 [SMILES: Cc1c(N2CN(c3ccc(=O)n(COP(=O)(O)O)c3C)C(=O)c3cc(F)c(Cl)cc32)ccc(F)c1F] | ¹H NMR (DMSO-d₆, 400 MHz) δ 11.0-12.2 (m, 2H), 7.78 (d, 1H, J = 9.3 Hz), 7.38 (br d, 2H, J = 9.8 Hz), 7.1-7.2 (m, 1H), 6.5-6.8 (m, 1H), 6.37 (d, 1H, J = 9.8 Hz), 5.6-5.8 (m, 2H), 4.6-5.5 (m, 2H), 2.2-2.4 (m, 3H), 2.20 (d, 3H, J = 2.4 Hz). |
| 5 | (5-(7-chloro-6-fluoro-1-(4- fluoro-2-methylphenyl)-4- oxo-1,4- dihydroquinazolin-3(2H)- yl)-6-methyl-2- oxopyridin-1(2H)- yl)methyl dihydrogen phosphate | 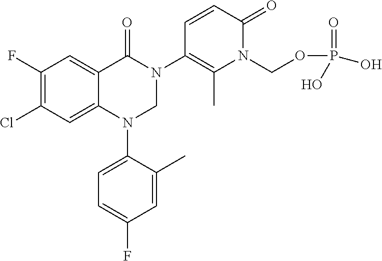 [SMILES: Cc1cc(F)ccc1N1CN(c2ccc(=O)n(COP(=O)(O)O)c2C)C(=O)c2cc(F)c(Cl)cc21] | ¹H NMR (DMSO-d6, 400 MHz) δ 7.78 (d, 1H, J = 9.3 Hz), 7.44 (d, 1H, J = 9.8 Hz), 7.3- 7.4 (m, 2H), 7.18 (br d, 1H, J = 6.8 Hz), 6.3- 6.5 (m, 2H, J = 9.8 Hz), 5.7-5.8 (m, 2H), 4.6- 5.6 (m, 2H), 2.8-4.3 (m, 2H), 2.3-2.4 (m, 3H), 2.26 (s, 3H), MS (m/z) 526 (M + H)⁺. |

TABLE 1A

| Compound | Name | Structure | Characterization |
|---|---|---|---|
| 1A | 7-fluoro-1-(4-fluoro-2- methylphenyl)-3-(2- methyl-6-oxo-1,6- dihydropyridin-3-yl)-6- (trifluoromethyl)-2,3- dihydroquinazolin-4(1H)- one | 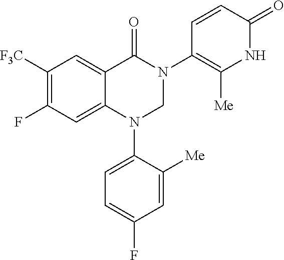 [SMILES: Cc1cc(F)ccc1N1CN(c2ccc(=O)[nH]c2C)C(=O)c2cc(C(F)(F)F)c(F)cc21] | [M + H]⁺ 450.1 ¹H NMR (DMSO-d6, 400 MHz) δ 11.78 (br s, 1H), 8.09 (d, 1H, J = 7.8 Hz), 7.3-7.5 (m, 3H), 7.2-7.2 (m, 1H), 6.1-6.2 (m, 2H), 4.8- 5.6 (m, 2H), 2.25 (s, 3H), 2.12 (s, 3H) |
| 2A | 3-(2,5-dimethyl-6-oxo-1,6- dihydropyridin-3-yl)-1-(2- methyl-4- (trifluoromethoxy)phenyl)- 6-(trifluoromethyl)-2,3- dihydropyrido[2,3- d]pyrimidin-4(1H)-one | 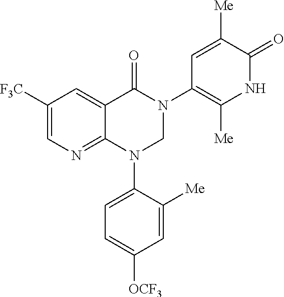 [SMILES: Cc1cc(OC(F)(F)F)ccc1N1CN(c2cc(C)c(=O)[nH]c2C)C(=O)c2cc(C(F)(F)F)cnc21] | [M + H]⁺ 513.1. ¹H NMR (DMSO-d₆, 400 MHz) δ 11.73 (s, 1H), 8.60 (d, 1H, J = 2.0 Hz), 8.31 (d, 1H, J = 2.0 Hz), 7.4-7.5 (m, 2H), 7.3-7.3 (m, 2H), 4.9-5.7 (m, 2H), 2.24 (s, 3H), 2.10 (br s, 3H), 1.94 (s, 3H) |
| 3A | 1-(3,4-difluoro-2- methylphenyl)-3-(2- methyl-6-oxo-1,6- dihydropyridin-3-yl)-6- (trifluoromethyl)-2,3- dihydropyrido[3,4- d]pyrimidin-4(1H)-one | 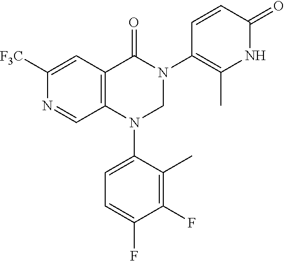 [SMILES: Cc1[nH]c(=O)ccc1N1CN(c2ccc(F)c(F)c2C)c2cnc(C(F)(F)F)cc2C1=O] | [M + H]⁺ 451.0. ¹H NMR (700 MHz, DMSO-d6) δ ppm 11.18-12.29 (m, 1 H) 8.00-8.07 (m, 1 H) 7.79-7.96 (m, 1 H) 7.43-7.51 (m, 1 H) 7.37-7.43 (m, 1 H) 7.2- 7.36 (m, 1 H) 6.21 (d, J = 9.47 Hz, 1H) 4.90-5.55 (m, 2 H) 2.19-2.29 (m, 3 H) 2.02-2.16 (m, 3 H) |
| 4A | 7-chloro-1-(3,4-difluoro-2- methylphenyl)-6-fluoro-3- (2-methyl-6-oxo-1,6- dihydropyridin-3-yl)-2,3- dihydroquinazolin-4(1H)- one | 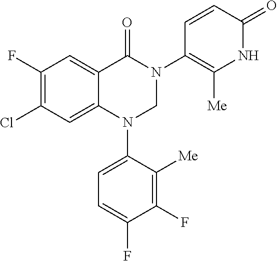 [SMILES: Cc1[nH]c(=O)ccc1N1CN(c2ccc(F)c(F)c2C)c2cc(Cl)c(F)cc2C1=O] | [M + 1]⁺ 434.1. ¹H NMR (DMSO-d6, 400 MHz) δ 11.75 (br s, 1H), 7.77 (d, 1H, J = 9.3 Hz), 7.35 (br dd, 2H, J = 10.3, 19.1 Hz), 7.13 (br s, 1H), 6.4-6.8 (m, 1H), 6.17 (br d, 1H, J = 9.3 Hz), 4.7-5.5 (m, 2H), 2.19 (br s, 1H), 2.0-2.1 (s, 3H |
| 5A | 7-chloro-6-fluoro-1-(4- fluoro-2-methylphenyl)-3- (2-methyl-6-oxo-1,6- dihydropyridin-3-yl)-2,3- dihydroquinazolin-4(1H)- one | 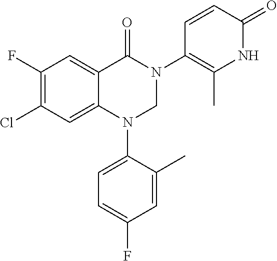 [SMILES: Cc1cc(F)ccc1N1CN(c2ccc(=O)[nH]c2C)C(=O)c2cc(F)c(Cl)cc21] | [M + 1]⁺ 416.3. ¹H NMR (DMSO-d6, 600 MHz): δ (ppm) 11.77 (br s, 1H), 7.76 (d, J = 9.1 Hz, 1H), 7.24-7.41 (m, 3H), 7.16 (br s, 1H), 6.25- 6.53 (m, 1H) 6.18 (d, J = 9.5 Hz, 1H), 4.69- 5.42 (m, 2H) 2.25 (s, 3H), 2.02-2.18 (m, 3H) |

### Example 6: Preparation of (5-(7-chloro-1-(3,4-difluoro-2-methylphenyl)-6-fluoro-4-oxo-1,4-dihydroquinazolin-3(2H)-yl)-6-methyl-2-oxopyridin-1(2H)-yl)methyl dihydrogen phosphate crystalline form

Crystalline forms of (5-(7-chloro-1-(3,4-difluoro-2-methylphenyl)-6-fluoro-4-oxo-1,4-dihydroquinazolin-3(2H)-yl)-6-methyl-2-oxopyridin-1(2H)-yl)methyl dihydrogen phosphate (compound Example No. 4) were prepared and characterized by X-ray powder diffraction (XRPD) using a Cu radiation source.

Route 1

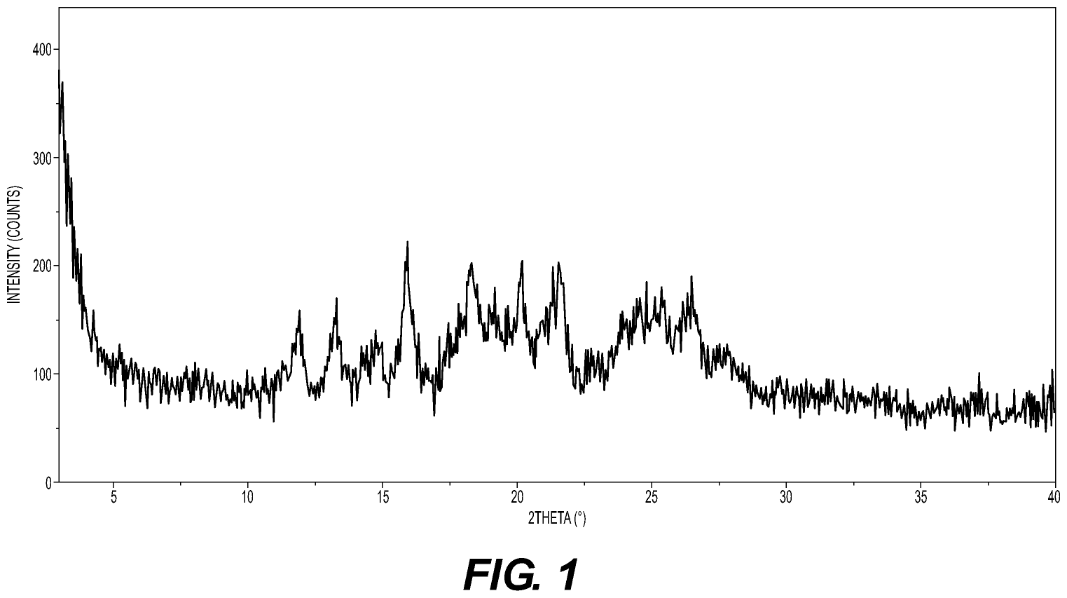

A solution of (5-(7-chloro-1-(3,4-difluoro-2-methylphenyl)-6-fluoro-4-oxo-1,4-dihydroquinazolin-3(2H)-yl)-6-methyl-2-oxopyridin-1(2H)-yl)methyl dihydrogen phosphate (35 mg, 0.064 mmol) was dissolved in ethyl acetate (161 μl) by heating to dissolution, then 10 drops of TBME were added and the solution allowed to stand at 25° C. for 4 days. A white solid formed and solvent was removed by evaporation to give (5-(7-chloro-1-(3,4-difluoro-2-methylphenyl)-6-fluoro-4-oxo-1,4-dihydroquinazolin-3(2H)-yl)-6-methyl-2-oxopyridin-1(2H)-yl)methyl dihydrogen phosphate (20.0 mg, 0.037 mmol, 57.1% yield) as a white solid, showing some crystalline character by XRPD (FIG. 1).

Route 2

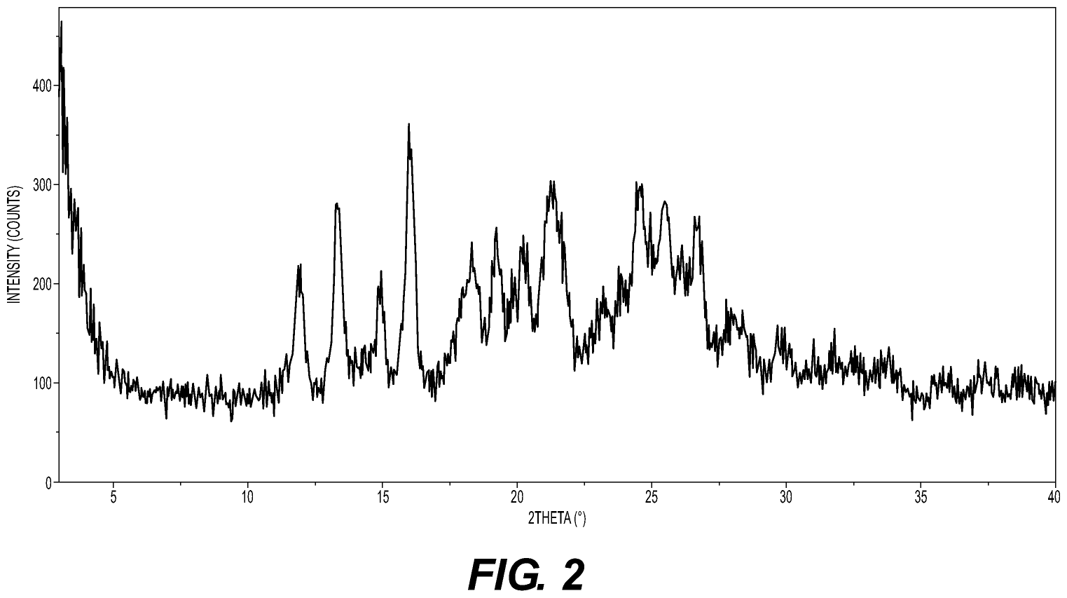

A solution of (5-(7-chloro-1-(3,4-difluoro-2-methylphenyl)-6-fluoro-4-oxo-1,4-dihydroquinazolin-3(2H)-yl)-6-methyl-2-oxopyridin-1(2H)-yl)methyl dihydrogen phosphate (875 mg, 1.61 mmol) in ethyl acetate (400 μl) was made by heating to 65° C., then t-butylmethyl ether (10 drops, 401 μl) was added, and left to stand and slowly cooled to 25° C. over 4 d. The formed solid was filtered and dried under air for 18 h then in high vacuum oven for 18 h at 60° C. The XPRD pattern (FIG. 2) showed crystalline character of the desired product (5-(7-chloro-1-(3,4-difluoro-2-methylphenyl)-6-fluoro-4-oxo-1,4-dihydroquinazolin-3(2H)-yl)-6-methyl-2-oxopyridin-1(2H)-yl)methyl dihydrogen phosphate (0.500 g, 0.919 mmol, 57.1% yield). HPLC/MS 0.74 min (Method B), [M+H]⁺ 544.0. ¹H NMR (DMSO-d₆, 400 MHz) δ 11.0-12.2 (m, 2H), 7.78 (d, 1H, J=9.3 Hz), 7.38 (br d, 2H, J=9.8 Hz), 7.1-7.2 (m, 1H), 6.5-6.8 (m, 1H), 6.37 (d, 1H, J=9.8 Hz), 5.6-5.8 (m, 2H), 4.6-5.5 (m, 2H), 2.2-2.4 (m, 3H), 2.20 (d, 3H, J=2.4 Hz).

Seeds Preparation

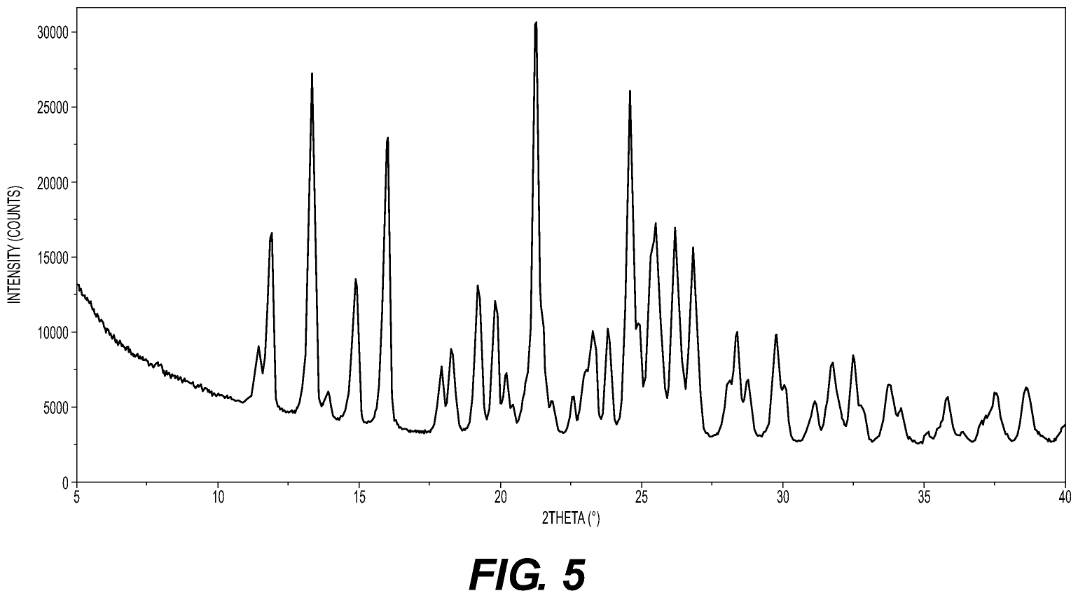

di-tert-butyl ((5-(7-chloro-1-(3,4-difluoro-2-methylphenyl)-6-fluoro-4-oxo-1,4-dihydroquinazolin-3(2H)-yl)-6-methyl-2-oxopyridin-1(2H)-yl)methyl) phosphate (8.5 g, 12.957 mmol, 1 equiv.) was charged with acetonitrile (59.5 mL, 0.218 M, 7 volume equivalents) and water (8.5 mL, 1.524 M, 1 volume equivalents). The mixture was warmed to Tr=50° C. (Tj=53° C.-54° C.) and stirred until a solution was obtained. Then, acetonitrile (34 mL, 0.381 M, 4 volume equivalents) was charged while maintaining the mixture temperature in the range of 45° C.-50° C. The mixture was then cooled to 15° C. at a rate of 0.4° C./min and stirred overnight. The mixture was then cooled further to 0° C. at a rate of 0.3° C./min and stirred for 2 h. The obtained suspension was filtered and washed with acetonitrile (2×3 vol). The wet cake was then vacuum dried at room temperature to afford 5.95 g of dry crystalline solid which was characterized by XRDP (FIG. 5). The obtained crystalline solid was used as seed in following procedures (Route 3 and Route 4).

Route 3

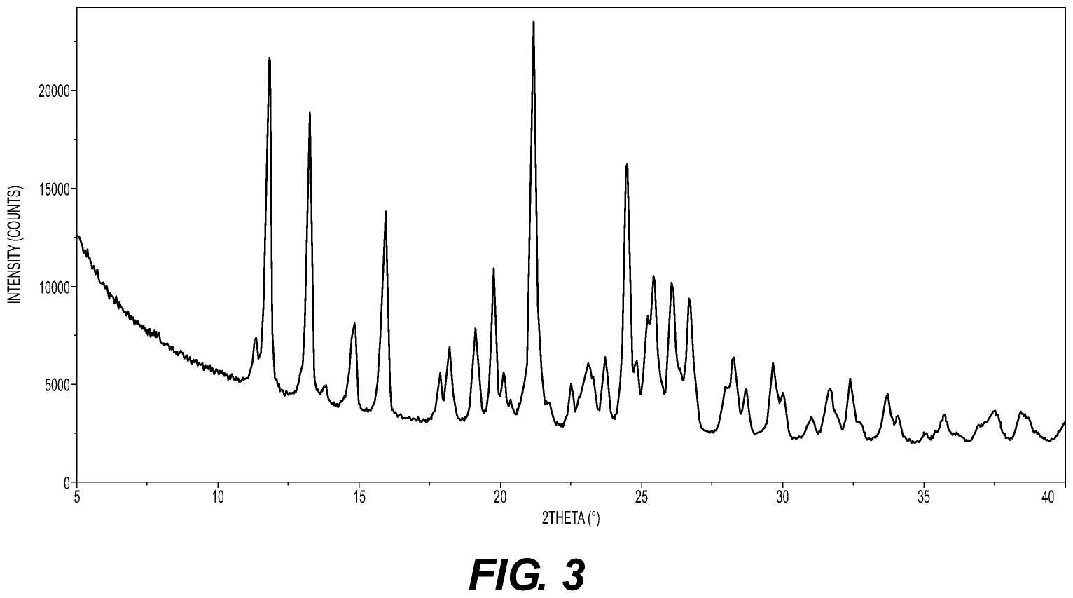

(5-(7-Chloro-1-(3,4-difluoro-2-methylphenyl)-6-fluoro-4-oxo-1,4-dihydroquinazolin-3(2H)-yl)-6-methyl-2-oxopyridin-1(2H)-yl)methyl dihydrogen phosphate was mixed with 7.4 volume equivalents of Acetone and 2.6 volume equivalents of DMSO at 25° C. Then 0.3 volume equivalents of DMSO were added and the mixture was stirred until full dissolution was achieved. A clarifying filtration was then performed. The following charges were based on dissolved compound. Three volume equivalents of water were added to the clarified solution, followed by 1.9 wt % of crystalline compound as seeds. The mixture was then mixed for around 15 h. An additional 3 volume equivalents of water were added followed by 1.2 wt % of crystalline compound as seeds. The mixture was then mixed for 40 min, then 8.26 volume equivalents of water were added over 6 h. The mixture was then aged for 19 h to afford a white suspension. The suspension was then filtered under vacuum and the cake was washed with 3×3 volume equivalents of water/acetone 2:1 (v:v) in sequence as displacement washes. The wet cake was then dried under vacuum with a slight sweep of air at approximately 50° C. until constant weight to afford crystalline (5-(7-chloro-1-(3,4-difluoro-2-methylphenyl)-6-fluoro-4-oxo-1,4-dihydroquinazolin-3(2H)-yl)-6-methyl-2-oxopyridin-1(2H)-yl)methyl dihydrogen phosphate (FIG. 3).

Route 4

(5-(7-Chloro-1-(3,4-difluoro-2-methylphenyl)-6-fluoro-4-oxo-1,4-dihydroquinazolin-3(2H)-yl)-6-methyl-2-oxopyridin-1(2H)-yl)methyl dihydrogen phosphate was mixed with 2.5 volume equivalents of acetone and 2.5 volume equivalents of DMSO at 25° C. until full dissolution was achieved. Then 5 volume equivalents of water were added followed by 16 wt % of crystalline compound as seeds, as a suspension in 10 volume equivalents of Acetone/DMSO/Water 1:1:2 (v:v:v) based on the seeds mass. The mixture was then mixed for 18 h to afford a white suspension. The suspension was then filtered under vacuum and the cake was washed with 3×3 volume equivalents of acetone in sequence as displacement washes. The wet cake was then dried under vacuum at approximately 50° C. until constant weight to afford crystalline (5-(7-chloro-1-(3,4-difluoro-2-methylphenyl)-6-fluoro-4-oxo-1,4-dihydroquinazolin-3(2H)-yl)-6-methyl-2-oxopyridin-1(2H)-yl)methyl dihydrogen phosphate. The XRPD pattern is shown in FIG. 4 and the characteristic peaks, or diffraction angles (2θ) of the XRPD pattern are listed in Table 2. XRPD was acquired using a Rigaku Miniflex X-ray Diffractometer with a Cu source using following measurement parameters:
Start Position (°2θ) 5.0000
End Position (°2θ) 40.0000
Step size 0.0200
Scan step Time (s) 0.3478
Anode material Cu
K-Alpha1 [Å] 1.54060
K-Alpha2 [Å] 1.54443
Generator settings 15 mA, 40 kV

TABLE 2
Characteristic peaks and d-spacings

| Pos. [°2θ] | d-spacing [Å] | Rel. Int. (%) |
|---|---|---|
| 7.9581 | 11.10998 | 1.56 |
| 11.347 | 7.7983 | 11.05 |
| 11.8794 | 7.44996 | 59.18 |
| 13.2901 | 6.66221 | 68.99 |
| 13.8326 | 6.40209 | 5.08 |
| 14.736 | 6.01159 | 13.27 |
| 14.8996 | 5.94596 | 19.67 |
| 15.9968 | 5.54053 | 46.94 |
| 17.9319 | 4.94675 | 11.06 |
| 18.2004 | 4.87435 | 17.81 |
| 19.1259 | 4.64053 | 22.28 |
| 19.2678 | 4.60667 | 14.18 |
| 19.8225 | 4.47901 | 36.24 |
| 20.1507 | 4.40679 | 12.42 |
| 20.4436 | 4.3443 | 5.26 |
| 21.2034 | 4.19033 | 100 |
| 21.7017 | 4.09522 | 6.94 |
| 22.5358 | 3.94549 | 10.97 |
| 22.7853 | 3.90285 | 8.49 |
| 23.1065 | 3.84932 | 18.47 |
| 23.3949 | 3.80252 | 9.54 |
| 23.6774 | 3.75779 | 17.41 |
| 24.482 | 3.63608 | 70.37 |
| 24.8436 | 3.58397 | 17.59 |
| 25.1774 | 3.5372 | 28.64 |
| 25.4492 | 3.50004 | 36.69 |
| 26.0887 | 3.41285 | 28.19 |
| 26.415 | 3.37423 | 14.15 |
| 26.678 | 3.34155 | 34.48 |
| 27.9924 | 3.18757 | 11.58 |
| 28.2445 | 3.15969 | 20.07 |
| 28.7578 | 3.10444 | 11.22 |
| 29.6739 | 3.01066 | 17.09 |
| 30.1037 | 2.96865 | 9.8 |
| 30.9981 | 2.885 | 5.95 |
| 31.6522 | 2.82686 | 12.44 |
| 32.0155 | 2.79561 | 5.03 |
| 32.4211 | 2.76155 | 15.9 |
| 32.833 | 2.72785 | 3.99 |
| 33.6916 | 2.65807 | 9.89 |
| 34.0801 | 2.63082 | 6.26 |
| 35.0199 | 2.56235 | 1.99 |
| 35.6245 | 2.52023 | 5.3 |
| 35.8221 | 2.50678 | 5.5 |
| 36.1438 | 2.48521 | 2.53 |
| 36.8816 | 2.43717 | 3.48 |
| 37.6252 | 2.39069 | 6.06 |
| 38.3991 | 2.34428 | 7.35 |
| 38.7594 | 2.32331 | 5.11 |
| 39.0517 | 2.30659 | 1.86 |
| 39.886 | 2.25838 | 2.87 |

Biological Assays

Biological Assay 1: Naᵥ1.8 Inhibitory Activity Human embryonic kidney 293 cells (HEK293) expressing human Naᵥ1.8, human Navol and human TREK1 (HEK293-Naᵥ1.8) were grown in T150 cell culture flasks at 37° C., 5% CO₂ incubator. HEK293-Naᵥ1.8 were passaged every 2-3 days when confluency reached 80-90% in T150 cell culture flasks.

Pharmacological assessment of compounds of the invention was performed using the QPatch 48 HTX electrophysiological platform. HEK293-Naᵥ1.8 cells were prepared on the day of use by removing culture media, washing in DPBS, adding Accutase (2 mL to cover the surface, aspirate 1 mL then 1-2 minutes at 37° C.) followed by addition of CHO-SFM II to stop the enzyme digestion and in order to obtain a suspension of 3×10⁶ cell/mL.

Compounds of the invention were prepared in an extracellular solution of the following composition: NaCl (145 mM), KCl (4 mM), CaCl₂) (2 mM), MgCl (2 mM), HEPES (1 mM), Glucose (10 mM), pH 7.4 with NaOH Osmolality 300 mOsM/L. An intracellular solution of the following composition was used: CsF (115 mM), CsCl (20 mm), NaCl (5 mM), EGTA (10 mM), HEPES (10 mM), Sucrose (20 mM), pH 7.2 with CsOH Osmolality 310 mOsm/L.

Utilizing the voltage-clamp mode in the QPatch 48 HTX system a half inactivation state voltage protocol (V_{1/2}) was used to determine pharmacological activity of compounds of the invention at Naᵥ1.8 ion channels. A V_{1/2} protocol was utilized with the following voltage steps: a holding voltage of −100 mV was established followed by a 20 ms voltage step to 0 mV (P1), followed by an inactivating voltage step at −46 mV for 8 seconds, followed by a step to −100 mV for 20 ms, before a 20 ms step to 0 mV (P2), then returned to the holding voltage of −100 mV. This voltage protocol was repeated at a frequency of every 15 seconds, the current magnitude was quantified at the P2 step throughout the recording. Inhibition of the measured current amplitude with compounds of the invention was analyzed by fitting a 4-6 points dose-response curve allowing determination of the fifty percent inhibition concentration (IC₅₀). P2 currents were normalized according to measurements made at baseline (Baseline, vehicle only), after compound (Input, at each test concentration), and after positive reference compound (FullResponse, to achieve complete block), fit to the following equation:

$$n . {I}_{CPD} = Normalized Current = \frac{( Input - Baseline )}{( FullResponse - Baseline )}$$

To assess current run-down over the course of the experiment vehicle-only wells were utilized and the normalized current with vehicle-only (n. I_{VEH}) was determined. To correct the compound response for run-down, the currents were corrected according to the following formula:

$$n . {I}_{RD\_Correct} = \frac{( n . {I}_{CPD} - n . {I}_{VEH} )}{( 1 - n . {I}_{VEH} )}$$

Compound inhibition was fitted to the following Hill Equation to estimate the half maximal inhibition concentration (IC₅₀)

$$Y = \frac{{C}^{h}}{( {IC}_{50}^{h} + {C}^{h} )}$$

where Y is the normalized run-down corrected inhibition relative to the vehicle control (equivalent to n. I_{RD_Correct}), C the test compound concentration, IC₅₀ the concentration of test compound to inhibit the sodium current by 50%, and h the Hill coefficient.

The exemplified compounds shown in Tables 1 and 1A herein are active against Nav1.8 sodium channels as measured using the assays described herein and as presented in Table 3 below.

Each of the listed compound Examples of the invention identified in the charts below, individually, was tested in at least one exemplified salt or free base form. Unless otherwise noted, the tested compound examples of the invention exhibited a pharmacological activity Nav1.8 pIC₅₀ (Qpatch)>5.0. In another aspect, tested compound examples of the invention exhibited a pharmacological activity Nav1.8 pIC₅₀ (Qpatch)>6.0.

TABLE 3
Results of Biological Assay 1

| Prodrug Compounds | | Parent Compounds | |
|---|---|---|---|
| Compound | | Parent | |
| Example No. | [Nav1.8] pIC50 | Compound No. | [Nav1.8] pIC50 |
| 1 | 7.5 | 1A | 8.0 |
| 2 | 6.3 | 2A | 7.7 |
| 3 | 5.9 | 3A | 7.0 |
| 4 | 7.5 | 4A | 8.3 |
| 5 | 7.2 | 5A | 8.3 |

Biological Assay 2: CAD Solubility

Kinetic solubility was measured using Charged Aerosol Detector (CAD). The aqueous kinetic solubility at pH 7.4 was determined by measuring the concentration of solute in solution after precipitation from DMSO stock solution. The DMSO stock solution was diluted 20-fold with phosphate buffered saline (PBS) pH 7.4 and the solubility of the compound was measured after 1 hour equilibration at room temperature by HPLC-CAD. Calibration standards of Ketoconazole and Primidone were prepared by serial dilutions in DMSO at concentrations ranging from 0.016 to 4.5 mg/ml to produce the calibration curve used to determine the solubility of the compounds as previously described in Max W. Robinson et al, Use of Calculated Physicochemical Properties to Enhance Quantitative Response When Using Charged Aerosol Detection, Anal. Chem., 2017, 89 (3), pp 1772-1777, which is herein incorporated by reference. CAD solubility of the prodrug compounds of the invention and the corresponding parent compounds was measured as described above and the results are shown in Table 4 below.

TABLE 4
Results of Biological Assay 2

| Prodrug Compounds | | Parent Compounds | |
|---|---|---|---|
| Compound | CAD solubility | Parent | CAD solubility |
| Example No. | (mg/mL) | Compound No. | (mg/mL) |
| 1 | ≥192 | 1A | 32 |
| 2 | ≥217 | 2A | 56 |
| 3 | 224 | 3A | ≥210 |
| 4 | 157 | 4A | 44 |
| 5 | 135 | 5A | 55 |

Biological Assay 3: Rat IV/PO Study

An in vivo rat pharmacokinetic study was conducted to determine whether prodrug compounds of the invention are converted to the respective parent compound upon administration. The rat pharmacokinetic study was conducted with a crossover design on two study days with a one-day recovery period between each study day. Three male, dual catheterized (femoral vein and carotid artery) Han Wistar rats were used for the study. Each rat was also implanted with a gastric catheter for oral dose administration. Rats were dosed at 1 mg/kg by a 60-minute intravenous (IV) infusion (femoral vein cannula), then subsequently oral dosed at 2 mg/kg via the gastric cannula, with 48 hours between dosing sessions. Dose solutions of the compound of Example 4 were prepared in 20% Cavitron/5% DMSO/75% water (IV) and in 6% Cavitron/5% DMSO/89% water (PO) without pH adjustment. The dose solutions were filtered using a 0.22 μm filter. The pH of the final dosing solutions was 6.0.

During the intravenous study leg, blood samples were collected from the carotid artery catheter at target times of 15, 30, 60 (end of infusion), 65, 75, 90, 120, 240, 360, 480, 720, and 1440 minutes following the initiation of the intravenous infusion of the compound of Example 4. During the oral study leg, blood samples were collected prior to dosing and at target times of 15, 30, 60, 90, 120, 180, 240, 360, 480, 720, and 1440 minutes following oral administration. Blood samples (100 μL) were mixed with 100 μL phosphatase inhibitor, a 50 μL aliquot of the blood and inhibitor mixture was transferred to a non-heparinized tube and stored at approximately −80° C. until analyzed. The concentrations in the filtered dose solutions were confirmed by preparing stepwise dilutions first into 50% aqueous acetonitrile with 0.10% formic acid then into heparinized male Wistar Han blood: inhibitor to achieve determined nominal concentrations. Triplicate 50 μL aliquots were removed and were frozen and stored at approximately 80° C. until analyzed by LC-MS/MS as described below. LC-MS/MS was used to quantify the compound of Example 4 and the corresponding parent compound of Example 4A in the biological samples generated in the above described in vivo study.

Samples were prepared by protein precipitation followed by LC-MS/MS analysis employing positive-mode ionization against a set of calibration standards for the compounds prepared in the same matrix. Pharmacokinetic parameters for the study were derived from the concentration versus time profiles. Key pharmacokinetic parameters such as AUC_{0-∞}. (extrapolated area under the blood concentration-time curve), AUC₀₋ₜ (area under the blood concentration-time curve to the last time point with quantifiable drug), Cmax (maximum concentration), Tmax (time Cmax is achieved), CL (systemic blood clearance), Vdss (steady-state volume of distribution), MRT (mean residence time), and t_{1/2} (half-life) were determined for the compound of Example 4. The key pharmacokinetic parameters such as AUC_{0-∞}, AUC₀₋ₜ, Cmax, Tmax, MRT, and t_{1/2} (half-life) were determined for the parent compound Example 4A. Descriptive statistical data of pharmacokinetic parameters were calculated, including the mean and standard deviation (SD) using Microsoft Excel. The data are shown below in Tables 5A and 5B. Data are reported as mean±SD (N=3).

TABLE 5A
Results of Biological Assay 3 (prodrug compound)

| | | Route | |
|---|---|---|---|
| | Parameter | Intravenous | Oral |
| Compound | Dose (mg/kg) | 1.2 ± 0.1 | 3.0 ± 0.0 |
| of | Cmax (ng/ml) | 238 ± 45 | There were no |
| Example 4 | Half-life (h) | 0.67 ± 0.29 | quantifiable |
| (Prodrug) | MRT (h) | 0.10 ± 0.02 | concentrations |
| | Cl (mL/min/kg) | 110 ± 33 | following oral |
| | Vdss (L/kg) | 0.687 ± 0.284 | administration |
| | AUC₀₋ₜ (mg · h/mL) | 0.197 ± 0.284 | |
| | AUC_{0-∞} (mg · h/mL)^{b} | 0.197 ± 0.044 | |
| | Bioavailability (%) | — | |

TABLE 5B
Results of Biological Assay 3 (parent compound)

| | | Route | |
|---|---|---|---|
| | Parameter | Intravenous | Oral |
| Compound | Cmax (ng/ml) | 308 ± 15 | 413 ± 21 |
| of | Half-life (h)^{b} | NR^{c}, 4.5, 4.8 | 6.0 ± 0.6 |
| Example | MRT (h)^{b} | NR^{c}, 6.7, 8.0 | — |
| 4A | AUC₀₋ₜ (mg · h/mL) | 1.75 ± 0.24 | 5.25 ± 1.60 |
| (Parent)^{d} | AUC_{0-∞} (mg · h/mL)^{b} | NR^{c}, 1.50, 1.94 | 5.71 ± 1.91 |

^{b}Values listed individually due to variability in IV arm
^{c}NR denotes “not recorded”
^{d}Data for Example 4A based on dosing of the Example 4 prodrug

Biological Assay 4: Manual Patch-Clamp Electrophysiology Assay

The pharmacological activity of the Compounds of Example 4A and Example 5A (active parent compound of prodrug Example 4 and Example 5, respectively) was investigated by a patch-clamp electrophysiological method using a cellular system in which human Nav1.8 was overexpressed in HEK293 cells. Using this approach Nav1.8 can be activated by modulating the plasma membrane voltage and then channel function (Na⁺ conduction) can be directly quantified. The ability of Nav1.8 inhibitors to block channel function in this system is a measurement of target binding and inhibition.

Methods

Cell preparation: HEK293 cells overexpressing human Nav1.8 (BIOCAT124824) were grown at 37° C., 5% C₀₂ in medium (DMEM/F12 with 10% FBS, 2 mM GlutaMAX, 0.1 mM NEAA and 400 mg/ml G418, 100 mg/ml Hygromycin-B and 0.625 mg/ml Puromycin). Cells were passaged every 2-3 days when confluency reached ˜80%.

Electrophysiological recording solution preparation: Extracellular and intracellular solutions were prepared and used for voltage-clamp recordings in the whole-cell patch-clamp study described below:
Extracellular solution for voltage-clamp recordings: NaCl (145 mM), KCl (5.4 mM), CaCl₂) (2 mM), MgCl₂ (1 mM), HEPES (10 mM), Glucose (5 mM); pH was adjusted to 7.4 with NaOH and osmolarity was adjusted to 310 mosM.
Intracellular solution for voltage-clamp recordings: Cs-methanesulfonate (85 mM), CsF (35 mM), CsCl (20 mM), NaCl (5 mM), EGTA (5 mM), HEPES (10 mM); pH was adjusted to 7.3 with CsOH and osmolarity was adjusted to 295 mosM.

Whole-cell patch-clamp study protocol: Whole-cell recordings were conducted at room temperature (22-25° C.) using MultiClamp700B amplifier connected to a Digidata 1550A interface controlled by Clampex10.6 software (Molecular Devices). The acquisition rate was 20 kHz and signals were filtered at 5 kHz. Patch electrodes were pulled with a P-1000 Flaming/Brown micropipette puller (Sutter Instruments, Novato, CA, USA). The recording electrodes had a resistance of 1-1.5 MΩ when filled with internal solution and access resistances were generally <3 MΩ after the formation of whole-cell configuration. Voltage errors were minimized with 80-90% series resistance compensation. The cells were continuously perfused with extracellular solution by gravity with a speed of 0.5 ml/min and solution suction was implemented by a pump at a speed of 35 rpm (Watson-Marlow 120U/DM2 peristaltic tube pump, Marlow, United Kingdom) to maintain a stable liquid level. A manually controlled fast-step perfusion system (SF-77B, Warner Instruments) was used for drug delivery.

In each recording, 0.1% DMSO was perfused for 2 min to monitor baseline and compound was applied for 6 min to ensure the inhibition reached the steady state.

Voltage-clamp recording protocol: Compound effects were tested using single-pulse resting protocol in which cells were depolarized to 0 mV for 50 ms from a holding voltage of −120 mV. The peak current amplitudes were measured from the activated currents at 0 mV. The stimulation was applied every 20 s.

Test Compounds: Compound was dissolved in DMSO at 10 mM as the maximal concentration. For dose-response study, stock solutions with concentrations ranging from 0.1 mM to 10 mM were prepared in DMSO. Different work concentrations were all produced in extracellular solution by 1:1000 diluting from each stock solution. For vehicle group, 0.1% DMSO in extracellular solution was used as negative control.

Nav1.8 current amplitudes measurement and normalization: Nav1.8 peak currents elicited by test pulse of 0 mV were measured and analysed by Clampfit 10.6 Software (Molecular Devices, USA). In each recording, all peak current amplitudes (I) acquired at different time points were normalized to the first data point (I₀) at time zero to obtain the I/I₀ values.

Compound inhibition analysis: The compound inhibition effect was calculated using the following equation:

% inhibition=([I/I₀]_{ctrl}−[I/I₀]_{cmpd})/[I/I₀]ctrl*100

[I/I₀]_{ctrl} is the average I/I₀ of the final 3 sweeps during baseline while applying 0.1% DMSO; [I/I₀]_{cmpd} is the average I/I₀ of the final 3 sweeps during compound application where inhibition reached the steady state.

Statistics and data visualization in GraphPad Prism: To generate graphs, normalized data and calculated inhibition rate form individual recordings were pasted into Prism 8.0 or 9.0 (Graphpad Software, San Diego, California, USA). The fittings of the dose-response curves were carried out with Prism using a four-parameter Hill's equation: E=(Eₘₐₓ−Eₘᵢₙ)/[1+(IC₅₀/C)h]+Emin, where E is the response, Eₘₐₓ and Eₘᵢₙ are the maximum and minimum response, respectively, there is no constraint on Eₘₐₓ and Eₘᵢₙ is constrained at zero. IC₅₀ is the concentration corresponding to half-maximal inhibitory effect, C is the drug concentration, and h is the hill coefficient or hill slope.

Results

The potency of compound Example 4A was tested in four separate experiments with three different synthetic batches and the potency of compound Example 5A was tested in a single experiment. The measured IC₅₀ (concentration at which 50% inhibition of Nav1.8 was observed) for each compound is reported in Table 6 below. The results demonstrate that the compound of Example 4A is a more potent inhibitor of Nav1.8 than the compound of Example 5A.

TABLE 6
Results of Manual Patch-Clamp Electrophysiology Assay

| Compound | IC₅₀ (nM) |
|---|---|
| Example 4Aª | 0.8 ± 0.2 |
| Example 5A^{b} | 2.2 |

^{a}Result reported is average of four separate experiments with three different synthetic batches of compound.
^{b}Result reported is from single experiment

1. A compound selected from the group consisting of: 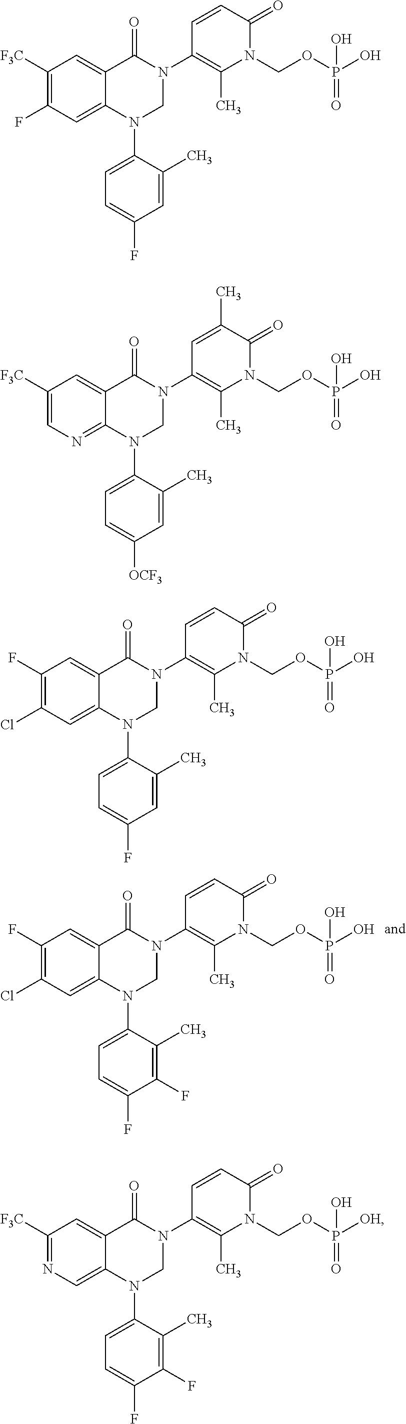
or a pharmaceutically acceptable salt thereof.

2. The compound of claim 1, being a pharmaceutically acceptable salt of the compound selected from the group consisting of: 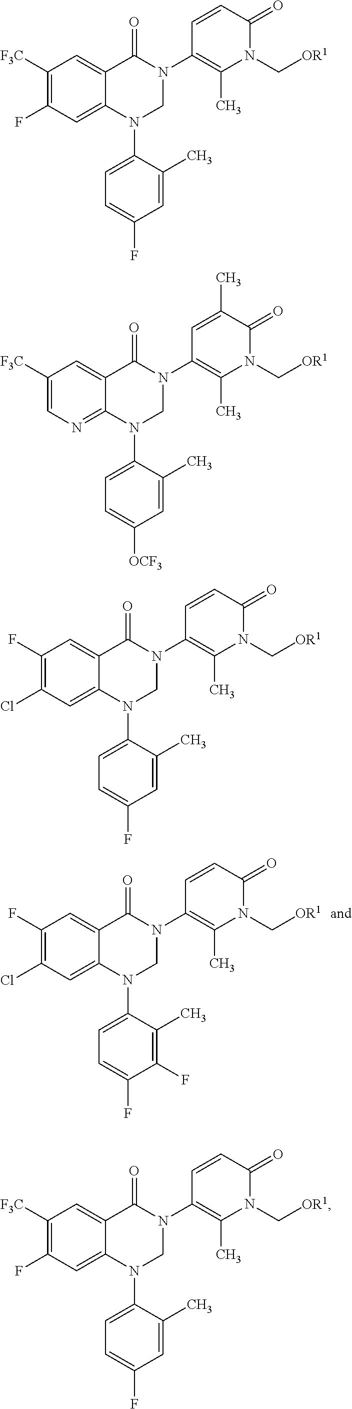
wherein:
R¹ is —P(O)(OH)O⁻M⁺, —PO(O⁻)₂·2M⁺, or —PO(O⁻)₂·D²⁺;
each M⁺ is independently a pharmaceutically acceptable monovalent cation; and
D²⁺ is a pharmaceutically acceptable divalent cation.

3. The compound of claim 1, which is: 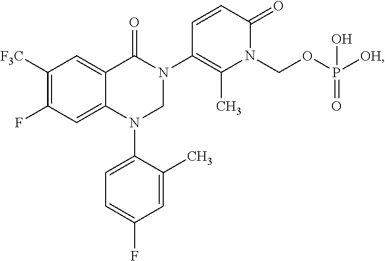 [SMILES: Cc1cc(F)ccc1N1CN(c2ccc(=O)n(COP(=O)(O)O)c2C)C(=O)c2cc(C(F)(F)F)c(F)cc21]
or a pharmaceutically acceptable salt thereof.

4. The compound of claim 1, which is: 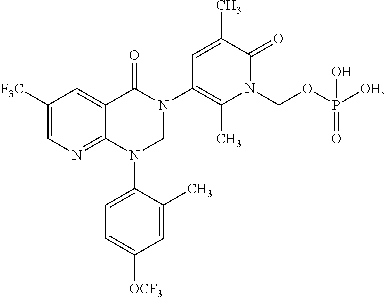 [SMILES: Cc1cc(OC(F)(F)F)ccc1N1CN(c2cc(C)c(=O)n(COP(=O)(O)O)c2C)C(=O)c2cc(C(F)(F)F)cnc21]
or a pharmaceutically acceptable salt thereof.

5. The compound of claim 1, which is: 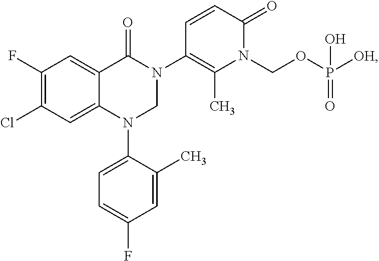 [SMILES: Cc1cc(F)ccc1N1CN(c2ccc(=O)n(COP(=O)(O)O)c2C)C(=O)c2cc(F)c(Cl)cc21]
or a pharmaceutically acceptable salt thereof.

6. The compound of claim 1, which is: 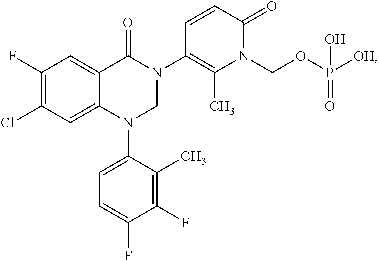 [SMILES: Cc1c(N2CN(c3ccc(=O)n(COP(=O)(O)O)c3C)C(=O)c3cc(F)c(Cl)cc32)ccc(F)c1F]
or a pharmaceutically acceptable salt thereof.

7. The compound of claim 1, which is: 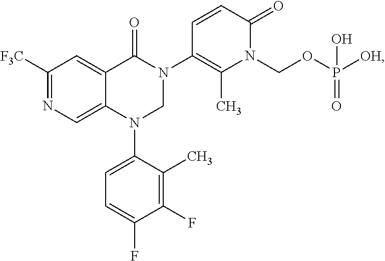 [SMILES: Cc1c(N2CN(c3ccc(=O)n(COP(=O)(O)O)c3C)C(=O)c3cc(C(F)(F)F)ncc32)ccc(F)c1F]
or a pharmaceutically acceptable salt thereof.

8. A compound selected from the group consisting of: 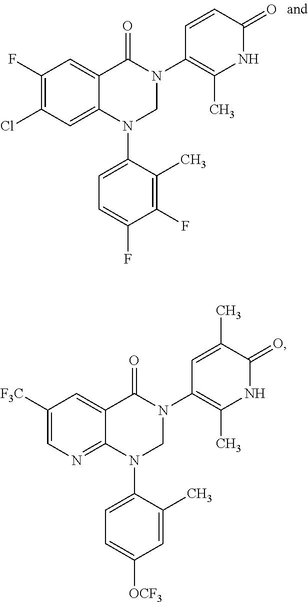 [SMILES: Cc1[nH]c(=O)ccc1N1CN(c2ccc(F)c(F)c2C)c2cc(Cl)c(F)cc2C1=O ; Cc1cc(OC(F)(F)F)ccc1N1CN(c2cc(C)c(=O)[nH]c2C)C(=O)c2cc(C(F)(F)F)cnc21]
or a tautomer thereof, or a pharmaceutically acceptable salt thereof.

9. A pharmaceutical composition comprising the compound or pharmaceutically acceptable salt thereof according to claim 1, and a pharmaceutically acceptable excipient.

10. A pharmaceutical composition comprising the compound, or tautomer thereof, or pharmaceutically acceptable salt thereof according to claim 8, and a pharmaceutically acceptable excipient.

11. The pharmaceutical composition according to claim 9, formulated for oral administration.

12. The pharmaceutical composition according to claim 9, formulated for intravenous administration.

13. A method of treatment of pain or a pain-associated disease in a human in need thereof, the method comprising administering to the human a compound according to claim 1, or a tautomer thereof, or a pharmaceutically acceptable salt thereof.

14. A method of treatment of atrial fibrillation in a human in need thereof, the method comprising administering to the human a compound according to claim 1, or a tautomer thereof, or a pharmaceutically acceptable salt thereof.

15.-19. (canceled)

20. The method according to claim 13, wherein the pain or pain-associated disease is neuropathic pain, ambulatory post-operative pain, or osteoarthritis.

21. A method of treatment of pain or a pain-associated disease in a human in need thereof, the method comprising administering to the human a compound according to claim 8, or a tautomer thereof, or a pharmaceutically acceptable salt thereof.

22. A method of treatment of atrial fibrillation in a human in need thereof, the method comprising administering to the human a compound according to claim 8, or a tautomer thereof, or a pharmaceutically acceptable salt thereof.

23. The method according to claim 21, wherein the pain or pain-associated disease is neuropathic pain, ambulatory post-operative pain, or osteoarthritis.

24. A method of treatment of pain or a pain-associated disease in a human in need thereof, the method comprising administering to the human a pharmaceutical composition according to claim 9.

25. A method of treatment of atrial fibrillation in a human in need thereof, the method comprising administering to the human a pharmaceutical composition according to claim 9.
